(19) United States
(12) Patent Application Publication
(10) Pub. No.: US 2009/0137828 A1
(43) Pub. Date: May 28, 2009

(54) Synthesis of 1A-Fluoro-25-Hydroxy-16-23E-Diene-26,27-Bishomo-20-Epi-Cholecalciferol

(75) Inventors: Milan R. Uskokovic, Upper Montclair, NJ (US); Stanislaw Marczak, Wayne, NJ (US); Ralf Loo, Groningen (NL); Marcel Van Der Sluis, Groningen (NL); Pawel Jankowski, Wayne, NJ (US); Hubert Maehr, Wayne, NJ (US)

Correspondence Address: Edwards Angell Palmer & Dodge LLP, P.O. Box 55874, Boston, MA, 02205, US

(73) Assignee: BIOXELL S.P.A., Milan (IT)

(21) Appl. No.: 12/063,955

(22) PCT Filed: Aug. 18, 2006
(86) PCT No.: PCT/US06/32381

§ 371 Date: Jun. 27, 2008

Related U.S. Application Data
(60) Provisional application No. 60/709,703, filed on Aug. 18, 2005.

(51) Int. Cl. C07C 401/00

(52) U.S. Cl. 552/653

(57) ABSTRACT

The invention provides a method of producing 20-methyl vitamin D3 compounds of formula (I). The method includes allylic and olefin oxidation, de-carbonylation, carbonyl reduction, fluoride substitution, epoxide deoxygenation, and Wittig-type couplings.

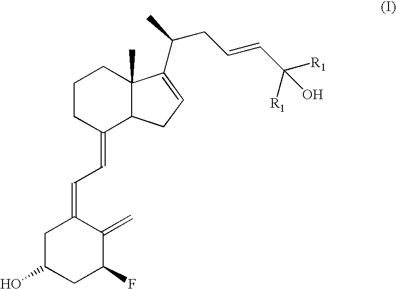

### RELATED APPLICATIONS

This application claims the benefit of U.S. provisional patent application Ser. No. 60/709,703 filed Aug. 18, 2005 (Attorney Docket No. 49949-63658P). The disclosure of the aforementioned provisional patent application is incorporated herein in its entirety by this reference.

### BACKGROUND OF THE INVENTION

The importance of vitamin D (cholecalciferol) in the biological systems of higher animals has been recognized since its discovery by Mellanby in 1920 (Mellanby, E. (1921) Spec. Rep. Ser. Med. Res. Council (GB) SRS 61:4). It was in the interval of 1920-1930 that vitamin D officially became classified as a “vitamin” essential for the normal development of the skeleton and maintenance of calcium and phosphorous homeostasis.

Studies involving the metabolism of vitamin D₃ were initiated with the discovery and chemical characterization of the plasma metabolite, 25-hydroxyvitamin D₃ [25(OH)D₃] (Blunt, J. W. et al. (1968) Biochemisty 6:3317-3322) and the hormonally active form, 1α,25(OH)₂D₃ (Myrtle, J. F. et al. (1970) J. Biol. Chem. 245:1190-1196; Norman, A. W. et al. (1971) Science 173:51-54; Lawson, D. E. M. et al. (1971) Nature 230:228-230; Holick, M. F. (1971) Proc. Natl. Acad. Sci. USA 68:803-804). The formulation of the concept of a vitamin D endocrine system was dependent upon the appreciation of the key role of the kidney in producing 1α,25(OH)₂D₃ in a carefully regulated fashion (Fraser, D. R. and Kodicek, E (1970) Nature 288:764-766; Wong, R. G. et al. (1972) J. Clin. Invest. 51:1287-1291), and the discovery of a nuclear receptor for 1 α,25(OH)₂D₃ (VD₃R) in the intestine (Haussler, M. R. et al. (1969) Exp. Cell Res. 58:234-242; Tsai, H. C. and Norman, A. W. (1972) J. Biol. Chem. 248:5967-5975).

The operation of the vitamin D endocrine system depends on the following: first, on the presence of cytochrome P450 enzymes in the liver (Bergman, T. and Postlind, H. (1991) Biochem. J. 276:427-432; Ohyama, Y. and Okuda, K. (1991) J. Biol. Chem. 266:8690-8695) and kidney (Henry, H. L. and Norman, A. W. (1974) J. Biol. Chem. 249:7529-7535; Gray, R. W. and Ghazarian, J. G. (1989) Biochem. J. 259:561-568), and in a variety of other tissues to effect the conversion of vitamin D₃ into biologically active metabolites such as 1α, 25(OH)₂D₃ and 24R,25(OH)₂D₃; second, on the existence of the plasma vitamin D binding protein (DBP) to effect the selective transport and delivery of these hydrophobic molecules to the various tissue components of the vitamin D endocrine system (Van Baelen, H. et al. (1988) Ann NY Acad. Sci. 538:60-68; Cooke, N. E. and Haddad, J. G. (1989) Endocr. Rev. 10:294-307; Bikle, D. D. et al. (1986) J. Clin. Endocrinol. Metab. 63:954-959); and third, upon the existence of stereoselective receptors in a wide variety of target tissues that interact with the agonist 1α,25(OH)₂D₃ to generate the requisite specific biological responses for this secosteroid hormone (Pike, J. W. (1991) Annu. Rev. Nutr. 11:189-216). To date, there is evidence that nuclear receptors for 1α,25(OH)₂D₃ (VD₃R) exist in more than 30 tissues and cancer cell lines (Reichel, H. and Norman, A. W. (1989) Annu. Rev. Med. 40:71-78).

Vitamin D₃ and its hormonally active forms are well-known regulators of calcium and phosphorous homeostasis. These compounds are known to stimulate, at least one of, intestinal absorption of calcium and phosphate, mobilization of bone mineral, and retention of calcium in the kidneys. Furthermore, the discovery of the presence of specific vitamin D receptors in more than 30 tissues has led to the identification of vitamin D₃ as a pluripotent regulator outside its classical role in calcium/bone homeostasis.

A paracrine role for 1α,25(OH)₂ D₃ (structure shown below) has been suggested by the combined presence of enzymes capable of oxidizing vitamin D₃ into its active forms, e.g., 25-OHD-1α-hydroxylase, and specific receptors in several tissues such as bone, keratinocytes, placenta, and immune cells. Moreover, vitamin D₃ hormone and active metabolites have been found to be capable of regulating cell proliferation and differentiation of both normal and malignant cells (Reichel, H. et al. (1989) Ann. Rev. Med. 40: 71-78).

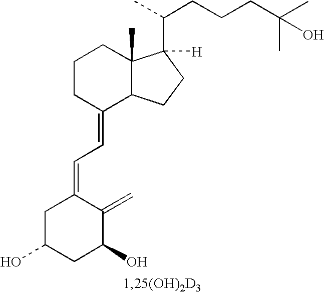

Thus, vitamin D₃ compounds exert a full spectrum of 1,25(OH)₂D₃ biological activities such as binding to the specific nuclear receptor VDR, suppression of the increased parathyroid hormone levels in 5,6-nephrectomized rats, suppression of INF-γ release in MLR cells, stimulation of HL-60 leukemia cell differentiation and inhibition of solid tumor cell proliferation (Uskokovic, M. R et al., “Synthesis and preliminary evaluation of the biological properties of a 1α,25-dihydroxyvitamin D₃ analogue with two side-chains.” Vitamin D: Chemistry, Biology and Clinical Applications of the Steroid Hormone; Norman, A. W., et al., Eds.; University of California: Riverside, 1997; pp 19-21; Norman et al., J. Med. Chem. 2000, Vol. 43, 2719-2730).

In both in vivo and cellular cultures, 1,25-(OH)₂D₃ undergoes a cascade of metabolic modifications initiated by the influence of 24R-hydroxylase enzyme. First 24R-hydroxy metabolite is formed, which is oxidized to 24-keto intermediate, and then 23S-hydroxylation and fragmentation produce the fully inactive calcitroic acid.

Given the activities of vitamin D₃ and its metabolites, much attention has focused on the development of synthetic analogs of these compounds. A large number of these analogs involves structural modifications in the A ring, B ring, C/D rings, and, primarily, the side chain (Bouillon, R. et al., Endocrine Reviews 16(2):201-204). Although a vast majority of the vitamin D₃ analogs developed to date involves structural modifications in the side chain, a few studies have reported the biological profile of A-ring diastereomers (Norman, A. W. et al. J. Biol. Chem. 268 (27): 20022-20030). Furthermore, biological esterification of steroids has been studied (Hochberg, R. B., (1998) Endocr Rev. 19(3): 331-348), and esters of vitamin D₃ are known (WO 97/11053).

Processes for manufacturing vitamin D analogs typically require multiple steps and chromatographic purifications. Previous processes have the disadvantages that, owing to the large number of process steps involved in the synthesis, they are very complex and lead to an unsatisfactory yield. See, Norman, A. W.; Okamura, W. H. PCT Int. Appl. WO 9916452 A1 990408; Chem. Abstr. 130:282223. Batcho, A. D.; Bryce, G. F.; Hennessy, B. M.; Iacobelli, J. A.; Uskokovic, M. R. Eur. Pat. Appl. EP 808833, 1997; Chem. Abstr. 128:48406. Nestor, J. J.; Manchand, P. S.; Uskokovic, M. R. Vickery, B. H. U.S. Pat. No. 5,872,113, 1997; Chem. Abstr. 130:168545.

For example, the synthesis of 1α-Fluoro-25-hydroxy-16-23E-diene-26,27-bishomo-20-epi-cholecalciferol (1)

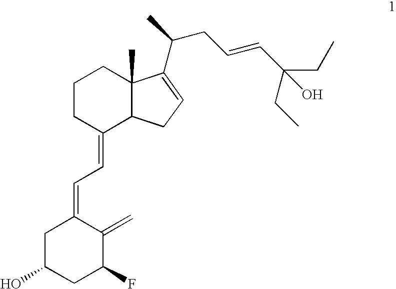

which may be utilized to treat a number of diseases including hyperproliferative skin diseases, neoplastic diseases, and sebaceous gland diseases (U.S. Pat. No. 5,939,408) has been accomplished. The synthesis starts from 1-(2-Hydroxy-1-methyl-ethyl)-7a-methyl-octahydro-inden-4-ol

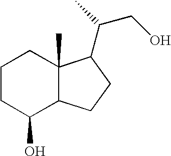

Steps of interest include a two-part addition of the side chain, including addition of an alkyne substitutent, followed by selective reduction to provide alkene side chains; and subsequent installation of the side chain quaternary carbon (carbon 25). The synthesis of the A-ring portion is not included.

Alternatively, the CD-ring portion has been synthesized by Daniewski et al. (U.S. Pat. No. 6,255,501) starting from 3a-Methyl-octahydro-1-oxa-cyclopropa[e]inden-4-one

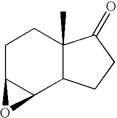

In this synthesis, a distinct starting material was utilized, presumably to allow for installation of a pre-synthesized side chain incorporating the alkene functionality. However, four additional steps were required to synthesize the alkene substituent, which included selective reduction of the corresponding alkyne, resulting in a synthesis of the CD-ring substituent over 11 steps. The A-ring portion is not included in the discussion.

The synthesis of the A-ring portion has been accomplished with modest success. One noteworthy example includes at least the steps of olefin epoxidation, allylic oxidation, and de-epoxidation, but suffers from low yields, side product formation, and difficult purifications. Other synthetic routes begin with (S)-carvone, and can be converted to the appropriate phosphine oxide over a multitude of steps. Other methodologies, including one starting from vitamin D3 and others starting from (S)-carvone, has proven to be more tedious.

The present invention provides an improved efficient synthesis of vitamin D compounds as compared to prior art syntheses.

### SUMMARY OF THE INVENTION

In one aspect, the invention provides a method of producing a vitamin D₃ compound of formula I

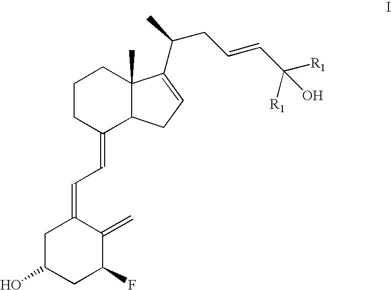

wherein each R₁ is independently alkyl; and pharmaceutically acceptable esters, salts, and prodrugs thereof; which comprises converting a compound of formula VI

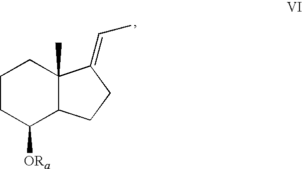

wherein Rₐ is a hydroxy protecting group, to a compound of formula X

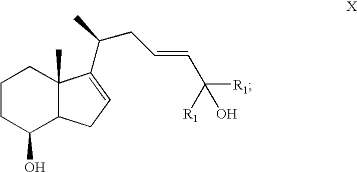

converting the compound of formula X to a compound of formula II

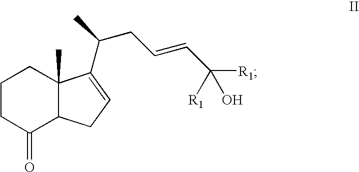

and

reacting the compound of formula II with a compound of formula III

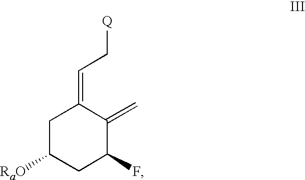

wherein Rₐ is defined as above and Q is a phosphorus-containing group; to thereby produce a compound of formula I.

In another aspect, the invention provides a method of producing a compound of formula X

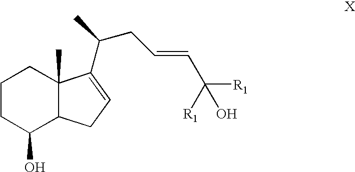

wherein each R₁ is independently alkyl; which comprises converting a compound of formula VI

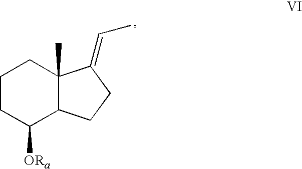

wherein Rₐ is a hydroxy protecting group, to a compound of formula VII

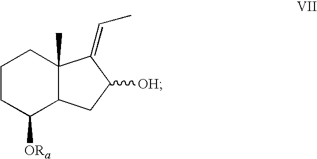

and
converting the compound of formula VII to a compound of formula X, to thereby produce a compound of formula X.

In still another aspect, the invention provides a method of producing a vitamin D₃ compound of formula I:

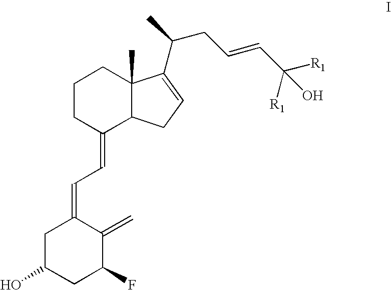

wherein each R₁ is independently alkyl; and pharmaceutically acceptable esters, salts, and prodrugs thereof; which comprises converting a compound of formula XII

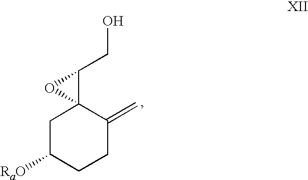

wherein Rₐ is a hydroxy protecting group, to a compound of formula XII-a

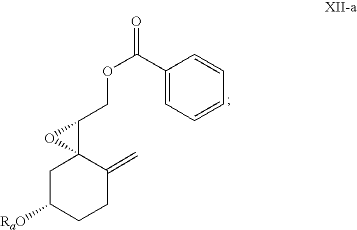

converting the compound of formula XII-a to a compound of formula XV

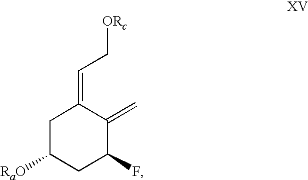

wherein R_{c} is H or benzoyl; converting the compound of formula XV to a compound of formula III

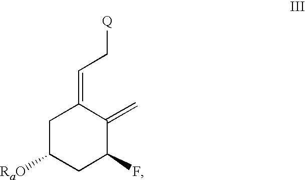

wherein Q is a phosphorus-containing group; and reacting the compound of formula III with a compound of formula II

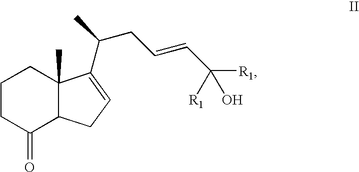

to thereby produce a compound of formula I.

In yet another aspect, the invention provides a method of producing a compound of formula XV

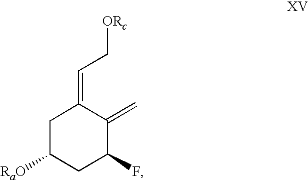

wherein Rₑ is H or benzoyl; which comprises converting a compound of formula XII

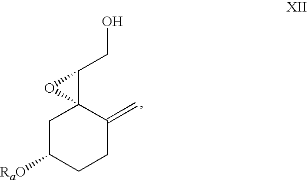

to a compound of formula XII-a

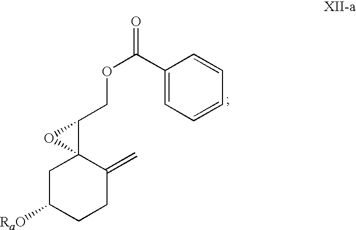

and

converting the compound of formula XII-a to a compound of formula XV, to thereby produce a compound of formula XV.

Another aspect of the invention provides a method of producing a vitamin D₃ compound of formula I

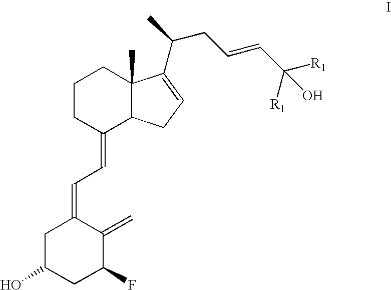

wherein:

each R₁ is independently alkyl;

and pharmaceutically acceptable esters, salts, and prodrugs thereof;

which comprises:

reacting a compound of formula II

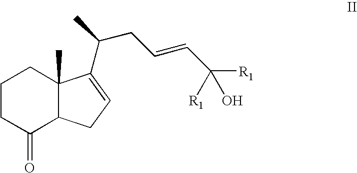

with a compound of formula III

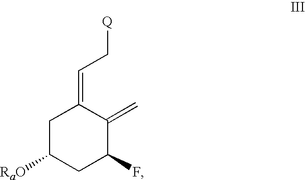

wherein Rₐ is defined as above and Q is a phosphorus-containing group in the presence of a strong base; to thereby produce a compound of formula I.

In another aspect, the invention provides the compound Acetic acid 1-ethylidene-2-hydroxy-7a-methyl-octahydro-inden-4-yl ester:

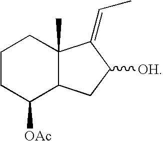

In still another aspect, the invention provides the compound Acetic acid 7a-methyl-1-(1-methyl-3-oxo-propyl)-3a,4,5,6,7,7a-hexahydro-3H-inden-4-yl ester:

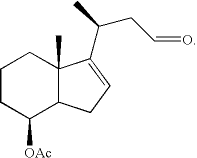

In yet another aspect, the invention provides the compound 5-(4-Acetoxy-7a-methyl-3a,4,5,6,7,7a-hexahydro-3H-inden-1-yl)-hex-2-enoic acid ethyl ester:

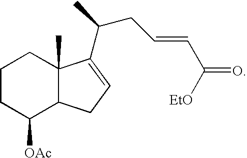

In another aspect, the invention provides the compound Benzoic acid 7-(tert-butyl-dimethyl-silanyloxy)-5-fluoro-4-methylene-1-oxa-spiro[2.5]oct-2-ylmethyl ester:

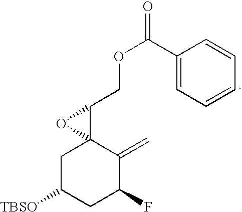

In still another aspect, the invention provides the compound Benzoic acid 2-[5-(tert-butyl-dimethyl-silanyloxy)-3-fluoro-2-methylene-cyclohexylidene]-ethyl ester:

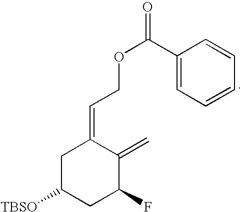

### DETAILED DESCRIPTION OF THE INVENTION

### 1. Definitions

Before further description of the present invention, and in order that the invention may be more readily understood, certain terms are first defined and collected here for convenience.

The terms “agent” and “reagent” are terms known to those of ordinary skill in the art. As used herein, each term is synonymous with the other.

The term “alkyl” refers to the radical of saturated aliphatic groups, including straight-chain allyl groups, branched-chain alkyl groups, cycloalkyl (alicyclic) groups, alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone, e.g., oxygen, nitrogen, sulfur or phosphorous atoms. In preferred embodiments, a straight chain or branched chain alkyl has 30 or fewer carbon atoms in its backbone (e.g., C₁-C₃₀ for straight chain, C₃-C₃₀ for branched chain), preferably 26 or fewer, and more preferably 20 or fewer. Likewise, preferred cycloalkyls have from 3-10 carbon atoms in their ring structure, and more preferably have 3, 4, 5, 6 or 7 carbons in the ring structure.

Moreover, the term alkyl as used throughout the specification and claims is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above.

An “alkylaryl” moiety is an alkyl substituted with an aryl (e.g., phenylmethyl (benzyl)).

The term “alkyl” also includes unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six, and still more preferably from one to four carbon atoms in its backbone structure, which may be straight or branched-chain. Examples of lower alkyl groups include methyl, ethyl, n-propyl, i-propyl, tert-butyl, hexyl, heptyl, octyl and so forth.

In preferred embodiment, the term “lower alkyl” includes a straight chain alkyl having 4 or fewer carbon atoms in its backbone, e.g., C₁-C₄ alkyl.

The terms “alkoxyalkyl,” “polyaminoalkyl” and “thioalkoxyalkyl” refer to alkyl groups, as described above, which further include oxygen, nitrogen or sulfur atoms replacing one or more carbons of the hydrocarbon backbone, e.g., oxygen, nitrogen or sulfur atoms.

The terms “alkenyl” and “alkynyl” refer to unsaturated aliphatic groups analogous in length and possible substitution to the alkyls described above, but that contain at least one double or triple bond, respectively. For example, the invention contemplates cyano and propargyl groups.

The term “aryl” as used herein, refers to the radical of aryl groups, including 5- and 6-membered single-ring aromatic groups that may include from zero to four heteroatoms, for example, benzene, pyrrole, furan, thiophene, imidazole, benzoxazole, benzothiazole, triazole, tetrazole, pyrazole, pyridine, pyrazine, pyridazine and pyrimidine, and the like. Aryl groups also include polycyclic fused aromatic groups such as naphthyl, quinolyl, indolyl, and the like. Those aryl groups having heteroatoms in the ring structure may also be referred to as “aryl heterocycles,” “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulflhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not mirror images of one another.

The term “deuteroalkyl” refers to alkyl groups in which one or more of the of the hydrogens has been replaced with deuterium.

The term “enantiomers” refers to two stereoisomers of a compound which are non-superimposable mirror images of one another. An equimolar mixture of two enantiomers is called a “racemic mixture” or a “racemate.”

The term “halogen” designates —F, —Cl, —Br or —I.

The term “haloalkyl” is intended to include alkyl groups as defined above that are mono-, di- or polysubstituted by halogen, e.g., fluoromethyl and trifluoromethyl.

The term “heteroatom” as used herein means an atom of any element other than carbon or hydrogen. Preferred heteroatoms are nitrogen, oxygen, sulfur and phosphorus.

The term “hydroxyl” means —OH.

The term “hydroxy-protecting group” signifies any group commonly used for the protection of hydroxy functions, such as for example, alkoxycarbonyl, acyl, alkylsilyl or alkylarylsilyl groups (hereinafter referred to simply as “silyl” groups), and alkoxyalkyl groups. Alkoxycarbonyl protecting groups include but are not limited to methoxycarbonyl, ethoxycarbonyl, propoxycarbonyl, isopropoxycarbonyl, butoxycarbonyl, isobutoxycarbonyl, tert-butoxycarbonyl, benzyloxycarbonyl or allyloxycarbonyl. The term “acyl” signifies an alkanoyl group of 1 to 6 carbons, in all of its isomeric forms, or a carboxyalkanoyl group of 1 to 6 carbons, such as an oxalyl, malonyl, succinyl, glutaryl group, or an aromatic acyl group such as benzoyl, or a halo, nitro or alkyl substituted benzoyl group. Alkoxyalkyl protecting groups include but are not limited to methoxymethyl, ethoxymethyl, methoxyethoxymethyl, or tetrahydrofuranyl and tetrahydropyranyl. Preferred silyl-protecting groups include but are not limited to trimethylsilyl, triethylsilyl, t-butyldimethylsilyl, dibutylmethylsilyl, diphenylmethylsilyl, phenyldimethylsilyl, diphenyl-t-butylsilyl and analogous alkylated silyl radicals.

The term “isomers” or “stereoisomers” refers to compounds which have identical chemical constitution, but differ with regard to the arrangement of the atoms or groups in space.

The term “obtaining” as in “obtaining a compound” is intended to include purchasing, synthesizing or otherwise acquiring the compound.

The term “phosphorous-containing reagent” refers to a reagent that contains phosphorus and can be reacted with a compound to provide the compound with a phosphorus-group. Compounds with phosphorus-containing groups can couple with compounds having carbonyl functionalities via, e.g., Wittig-type reactions, to provide compounds with alkene and alkyne groups. Typical phosphorous containing reagents used to make Wittig-type reagents include, but are not limited to, triphenylphosphine, trialkylphosphine, diphenylphosphine oxide, and triethyl phosphonoacetate.

The terms “polycyclyl” or “polycyclic radical” refer to the radical of two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfilydryl, alkylthio, arylthio, thiocarboxylate, sulfates, sulfonato, sulfamoyl, sulfonamido, nitro, trifluoromethyl, cyano, azido, heterocyclyl, alkyl, alkylaryl, or an aromatic or heteroaromatic moiety.

The term “prodrug” includes compounds with moieties which can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs which are converted to active forms through other mechanisms in vivo are also included.

The term “secosteroid” is art-recognized and includes compounds in which one of the cyclopentanoperhydro-phenanthrene rings of the steroid ring structure is broken. 1α,25(OH)₂D₃ and analogs thereof are hormonally active secosteroids. In the case of vitamin D₃, the 9-10 carbon-carbon bond of the B-ring is broken, generating a seco-B-steroid. The official IUPAC name for vitamin D₃ is 9,10-secocholesta-5,7,10(19)-trien-3B-ol. For convenience, a 6-s-trans conformer of 1α,25(OH)₂D₃ is illustrated herein having all carbon atoms numbered using standard steroid notation.

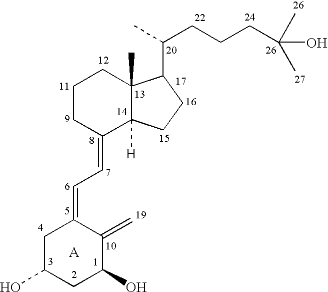

In the formulas presented herein, the various substituents on ring A are illustrated as joined to the steroid nucleus by one of these notations: a dotted line [symbol] indicating a substituent which is in the β-orientation (i.e., above the plane of the ring), a wedged solid line [symbol] indicating a substituent which is in the α-orientation (i.e., below the plane of the molecule), or a wavy line [symbol] indicating that a substituent may be either above or below the plane of the ring. In regard to ring A, it should be understood that the stereochemical convention in the vitamin D field is opposite from the general chemical field, wherein a dotted line indicates a substituent on Ring A which is in an α-orientation (i.e., below the plane of the molecule), and a wedged solid line indicates a substituent on ring A which is in the β-orientation (i.e., above the plane of the ring). As shown, the A ring of the hormone 1α,25(OH)₂D₃ contains two asymmetric centers at carbons 1 and 3, each one containing a hydroxyl group in well-characterized configurations, namely the 1α- and 3β-hydroxyl groups. In other words, carbons 1 and 3 of the A ring are said to be “chiral carbons” or “carbon centers”.

Furthermore the indication of stereochemistry across a carbon-carbon double bond is also opposite from the general chemical field in that “Z” refers to what is often referred to as a “cis” (same side) conformation whereas “E” refers to what is often referred to as a “trans” (opposite side) conformation. As shown, the A ring of the hormone 1-alpha,25(OH)₂D₃ contains two asymmetric centers at carbons 1 and 3, each one containing a hydroxyl group in well-characterized configurations, namely the 1-alpha- and 3-beta-hydroxyl groups. In other words, carbons 1 and 3 of the A ring are said to be “chiral carbons” or “chiral carbon centers.” Regardless, both configurations, cis/trans and/or Z/E are encompassed by the compounds of the present invention. With respect to the nomenclature of a chiral center, the terms “d” and “1” configuration are as defined by the IUPAC Recommendations. As to the use of the terms, diastereomer, racemate, epimer and enantiomer, these will be used in their normal context to describe the stereochemistry of preparations.

The term “subject” includes organisms which are capable of suffering from a vitamin D₃ associated state or who could otherwise benefit from the administration of a vitamin D₃ compound of the invention, such as human and non-human animals. Preferred human animals include human patients suffering from or prone to suffering from a vitamin D₃ associated state, as described herein. The term “non-human animals” of the invention includes all vertebrates, e.g., mammals, e.g., rodents, e.g., mice, and non-mammals, such as non-human primates, sheep, dog, cow, chickens, amphibians, reptiles, etc.

The term “sulfhydryl” or “thiol” means —SH.

With respect to the nomenclature of a chiral center, terms “d” and “1”, and “R” and “S” configurations are as defined by the IUPAC Recommendations. As to the use of the terms, diastereomer, racemate, epimer and enantiomer will be used in their normal context to describe the stereochemistry of preparations.

### 2. Overview of Syntheses of the Invention

The synthesis of the vitamin D₃ analogue 1, shown below in Scheme 1, has been previously reported in the literature (Radinov et al. J. Org. Chem. 2001, 66, 6141; Daniewski et al. U.S. Pat. No. 6,255,501; Batcho et al. U.S. Pat. No. 5,939,408). The prior art synthesis of vitamin D₃ analogue 1 requires 28 process steps. In contrast, the improved synthesis of the instant invention, as embodied in Schemes 2-4 below, provides the total synthesis of vitamin D₃ analogue 1, in one embodiment, in 19 steps and, in another embodiment, 21 steps.

As shown in Schemes 1-4, the synthesis of vitamin D₃ analogue 1 in accordance with the invention includes starting material cleavage, allylic oxidation, rearrangements, chain length extension, selective 1,2-addition, and Horner-Wittig coupling. Although the invention is described by reference to Schemes 1-4, which exemplify a specific embodiment of the synthesis of vitamin D₃ analogue 1, a number of vitamin D₃ compounds can be synthesized using the methods described in this section and the following working examples without undue experimentation.

Scheme 1 provides a summary of the conversion of vitamin D₂ (2) to compound 1. Compound 2 was initially hydroxyl protected. Oxidation with ozone, followed by a reductive workup provided intermediates 3 and 4. The conversion of 4 to 6 took place over eight steps, and included olefin epoxidation, allylic oxidation, and deoxygenation. The conversion of 3 to 5 was accomplished over eight steps and included allylic oxidation and rearrangement, and chain elongation. The final coupling of 5 and 6 took place under standard Horner-Wittig conditions to complete the novel synthesis of 1.

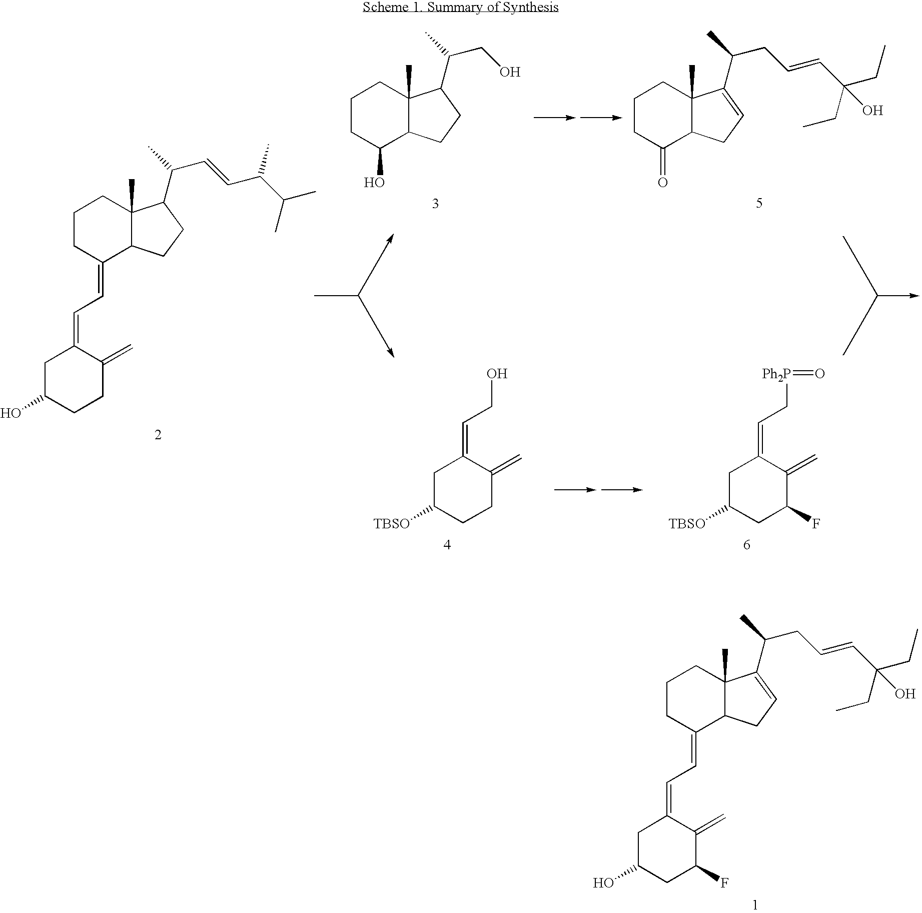

Scheme 2 outlines the cleavage of compound 2 to synthetic precursors 3 and 4. The hydroxyl group of 2 was initially protected with a t-butyl dimethyl silyl group, and ozonolysis was followed by a reductive workup with sodium borohydride to provide diol 3 in 60% yield, and alcohol 4 in 40% yield.

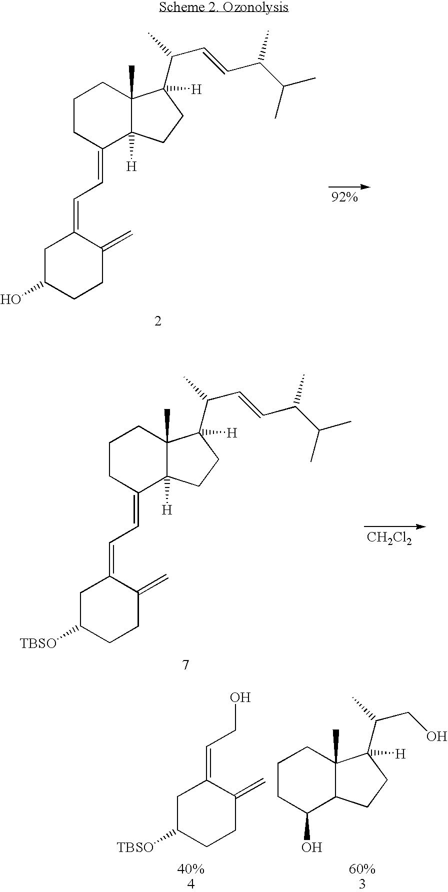

In another embodiment, compound 2 can be cleaved in the first step to provide compound 3 and compound 4a, which is followed by a two step protection to provide compound 4 (Scheme 2a).

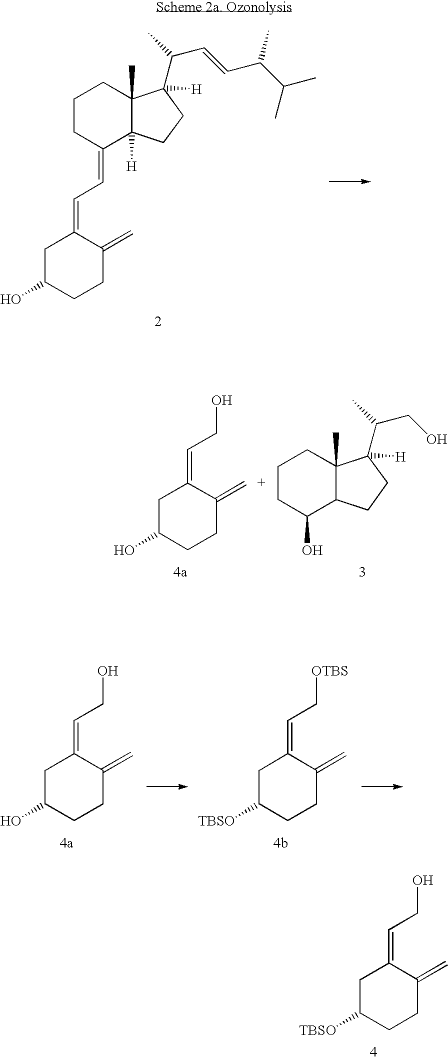

Scheme 3 details the conversion of 4 to the A-ring phosphine oxide 6. Compound 4 was epoxidized at the trisubstituted olefin in the presence of mCPBA in methylene chloride to provide 8 in 84% yield. Benzoyl protection of the primary hydroxyl group provided compound 9 in 91% yield, and was followed by allylic oxidation in the presence of selenium dioxide and t-butyl hydrogen peroxide in dioxane to give 10 as a mixture of epimeric compounds. The preferred isomer was reacted with diethylaminosulfur trifluoride (DAST) to provide fluorinated 11 in 75% yield. The conversion of 11 to 12 was accomplished in 61% yield in the presence of tris(3,5-dimethylpyrazoyl)hydridoborate rhenium trioxide and triphenyl phosphine in a sealed tube at 100° C. over 14 h. Benzyl hydrolysis in sodium methoxide solution provided hydroxyl compound 13 in 73% yield. The hydroxyl group of 13 was converted to the chloride compound 21 in the presence of triphosgene and pyridine, and subsequently converted to the Horner-Wittig reagent 6 by substitution of the chloride with diphenyl phosphine oxide. The conversion of 13 to 6 was accomplished in 76% yield.

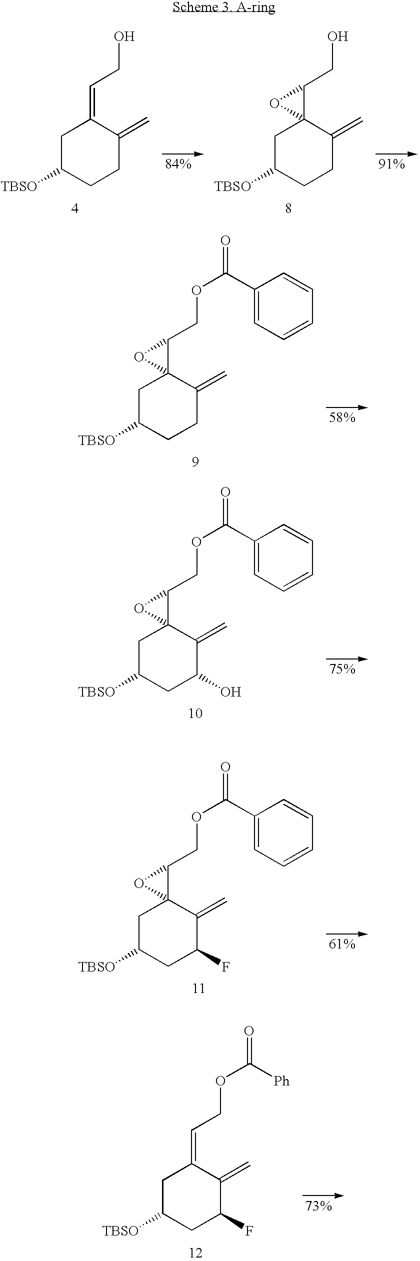

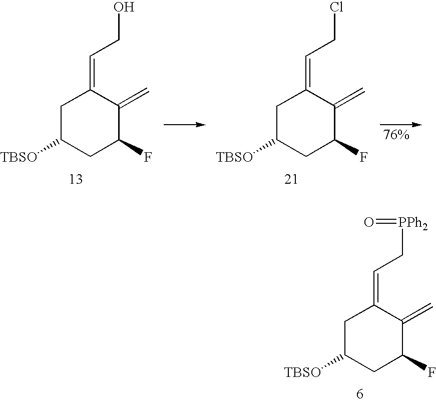

In another embodiment, the conversion of 11 to 13 takes place via a tungsten chloride mediated olefination of 11, which also deprotects the primary alcohol to yield 13a. Epimerization of 13a with radiation and 9-fluoronone provided compound 13 in a distinct two step procedure (Scheme 3a).

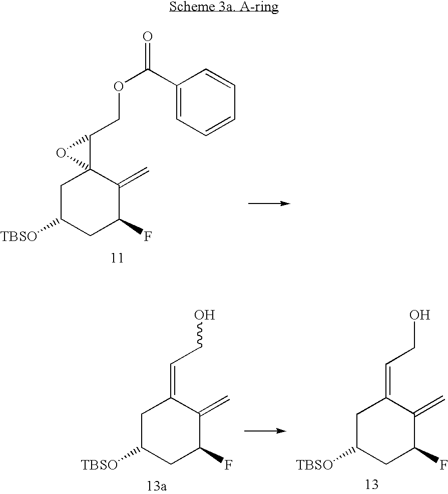

Scheme 4 describes the conversion of diol 3 to precursor 5. Compound 3 was oxidized to aldehyde 14 in 89% yield in the presence of TEMPO and NCS. The hydroxyl group was acetate protected to provide 15, and converted to the alkene mixture 16 in the presence of palladium and benzalacetone. Allylic oxidation provided an isomeric mixture of alcohols 17, which was subsequently subjected to Claisen rearrangement conditions to produce aldehyde 18 in 60% yield. Surprisingly, both isomers of 17 provided one isomer of 18. Chain elongation via a Wittig-Horner coupling provided ester 19 in high yield. Reduction of the ester with ethyl grignard in the presence of cerium trichloride provided diol 20 in 99% yield. The oxidation of 20 in the presence of PDC provided intermediate 5.

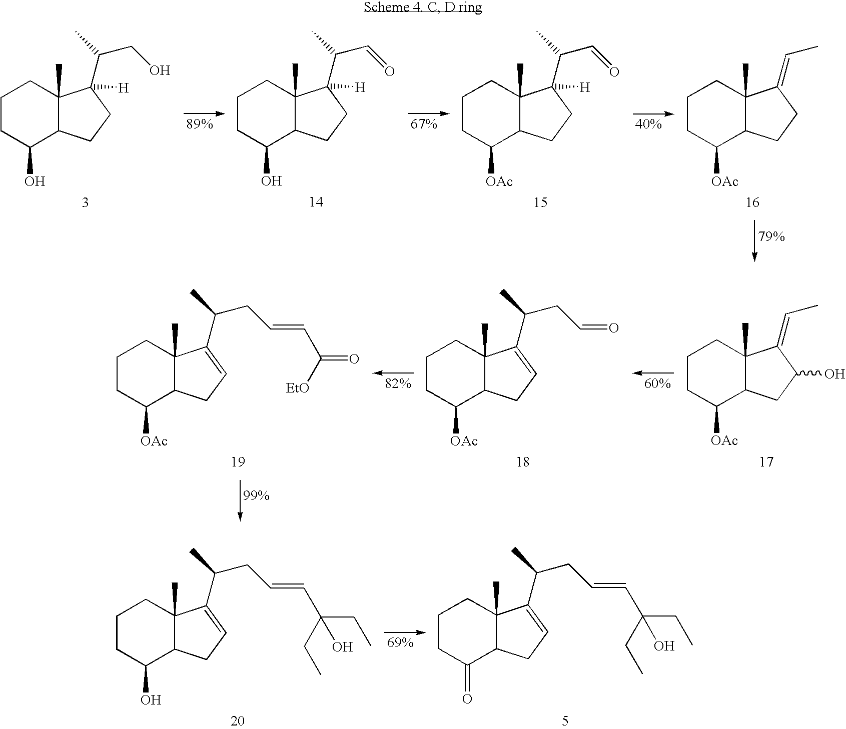

Compound 3 can also be converted to 15 by an initial acetate protection of the ring alcohol to produce 3a, followed by oxidation of the primary alcohol under Swern conditions (Scheme 4a).

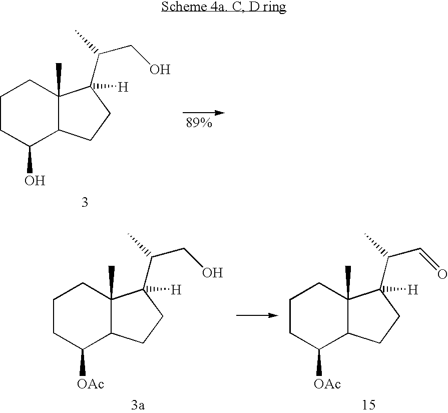

The conversion of 15 to 16 (schemes 4 and 4a) was accomplished, although a number of olefin side products were observed. Because purification of 16 is tedious and involves the use of medium pressure silver nitrate impregnated silica gel column chromatography, the product mixture was utilized in the next step. The reaction mixture was subsequently subjected to oxidation conditions, wherein compound 17 and other oxidation products could be separated by column chromatography. Interestingly, the over-oxidized side product (ketone) could be converted to 17 by reaction with a reducing agent, notably NaBH₄.

In one embodiment, compound 5 was further protected with a trimethyl silyl group, and then coupled with 6 in the presence of base (Scheme 5). The silyl protecting groups were removed in the presence of tetrabutyl ammonium fluoride (TBAF) to afford 1. The yield of 1 was 74% starting from the silyl protected 5. In another embodiment, compound 5 was coupled with 6 in the presence of base, followed by in situ deprotection of the silyl group with tetrabutyl ammonium fluoride (TBAF) to afford 1 (Scheme 5). The second embodiment therefore provides a one-step, one-pot synthesis of 1 starting from 5 and 6.

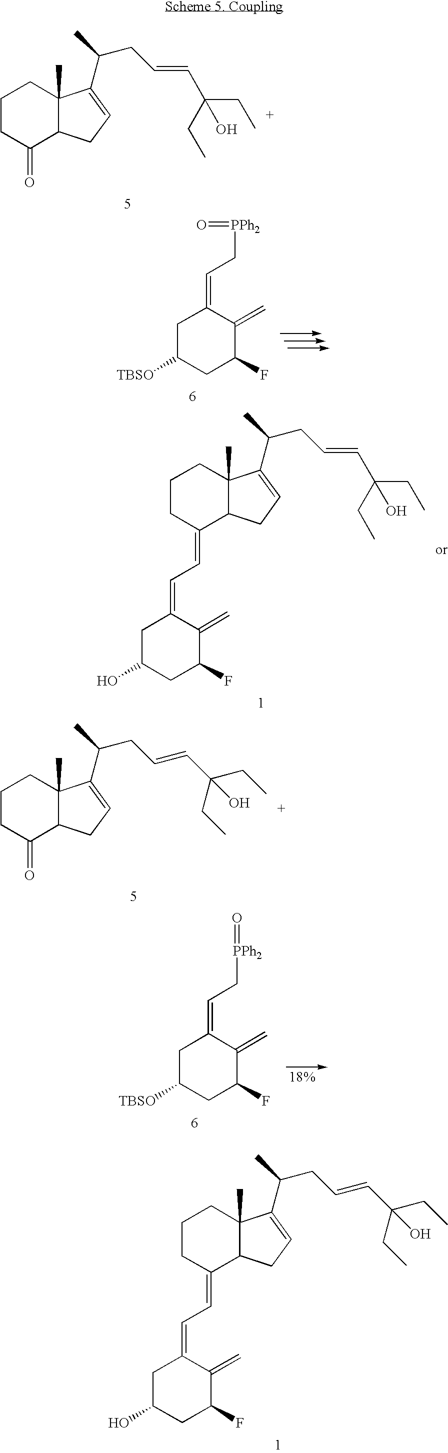

The invention therefore provides for the conversion of a compound of formula IV to a compound of formula II (CD-ring portion) in eight steps. Additionally, seven of the eight steps provide reaction products in yields of 60-99%, demonstrating the efficacy of the synthetic route. The invention also provides the A-ring portion in eight steps starting from the vitamin D₂ cleavage product 4. Including the coupling steps of 5 and 6, the invention provides for a novel 19-step synthesis of 1. Alternatively, the invention also provides for a 21-step synthesis of 1. The current methodology represents a significant simplification of the protocol described and practiced previously which required 28 steps.

Chiral syntheses can result in products of high stereoisomer purity. However, in some cases, the stereoisomer purity of the product is not sufficiently high. The skilled artisan will appreciate that the separation methods described herein can be used to further enhance the stereoisomer purity of the vitamin D₃-epimer obtained by chiral synthesis.

Naturally occurring or synthetic isomers can be separated in several ways known in the art. Methods for separating a racemic mixture of two enantiomers include chromatography using a chiral stationary phase (see, e.g., “Chiral Liquid Chromatography,” W. J. Lough, Ed. Chapman and Hall, New York (1989)). Enantiomers can also be separated by classical resolution techniques. For example, formation of diastereomeric salts and fractional crystallization can be used to separate enantiomers. For the separation of enantiomers of carboxylic acids, the diastereomeric salts can be formed by addition of enantiomerically pure chiral bases such as brucine, quinine, ephedrine, strychnine, and the like. Alternatively, diastereomeric esters can be formed with enantiomerically pure chiral alcohols such as menthol, followed by separation of the diastereomeric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomeric salts.

### 3. Description of Certain Embodiments of the Invention

The subject invention will now be described in terms of certain preferred embodiments. These embodiments are set forth to aid in understanding the invention but are not to be construed as limiting.

The subject invention is concerned generally with a stereospecific and regioselective process for preparing vitamin D₃ compounds of formula I. Thus, in one aspect, the invention provides a method of producing a vitamin D₃ compound of formula I

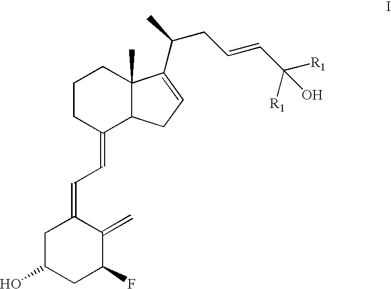

wherein each R₁ is independently alkyl; and pharmaceutically acceptable esters, salts, and prodrugs thereof; which comprises converting a compound of formula VI

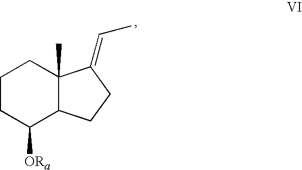

wherein Rₐ is a hydroxy protecting group, to a compound of formula X

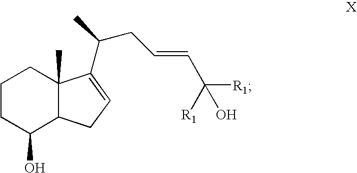

converting the compound of formula X to a compound of formula II

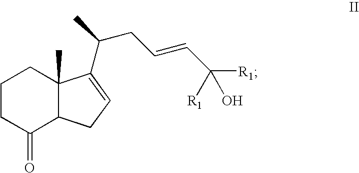

and
reacting the compound of formula II with a compound of formula III

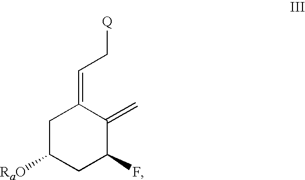

wherein Rₐ is defined as above and Q is a phosphorus-containing group; to thereby produce a compound of formula I.

In another aspect, the invention provides a method of producing a compound of formula X

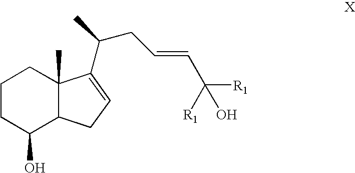

wherein each R₁ is independently alkyl; which comprises converting a compound of formula VI

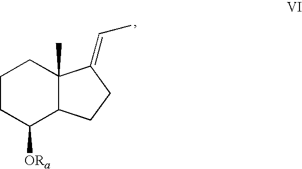

wherein Rₐ is a hydroxy protecting group, to a compound of formula VII

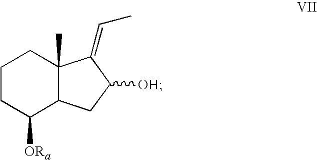

and

converting the compound of formula VII to a compound of formula X, to thereby produce a compound of formula X.

In one embodiment, the invention provides a method, further comprising reacting the compound of formula VI

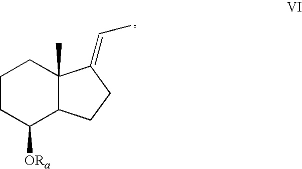

wherein Rₐ is a hydroxy protecting group, with an oxidation reagent to form a compound of formula VII

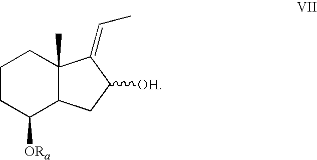

In another embodiment, the invention provides a method, further comprising subjecting the compound of formula VII

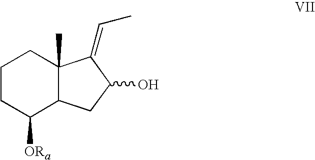

wherein Rₐ is a hydroxy protecting group; to rearrangement conditions to form a compound of formula VIII

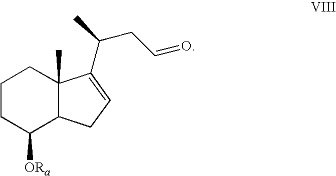

In still another embodiment, the invention provides a method, further comprising reacting the compound of formula VIII

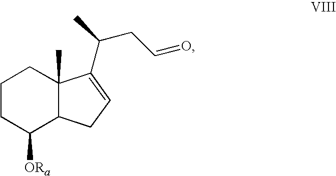

with a phosphorous-containing reagent of formula VIII-a

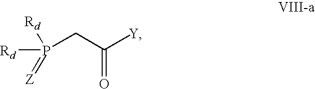

wherein Z is oxygen or absent; Y is OR_{b}, NR_{b}R_{b}, or S(O)ₙR_{b}; each R_{d} is independently alkyl, aryl, or alkoxy; each R_{b} is independently H, alkyl, or aryl; and n is 0-2; in the presence of a base to form a compound of formula IX

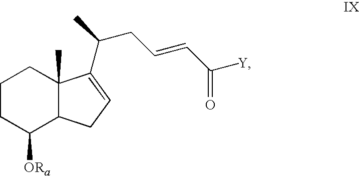

wherein Rₐ and Y are as defined above.

In yet another embodiment, the invention provides a method, further comprising reacting the compound of formula IX

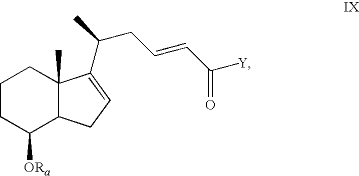

with an organometallic reagent to form a compound of formula X

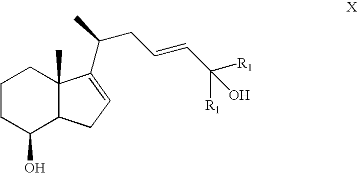

wherein each R₁ is independently alkyl.

In one embodiment, the invention provides the oxidation reagent comprising selenium dioxide (SeO₂) and t-butylhydrogenperoxide.

In another embodiment, the invention provides a method, wherein the rearrangement condition comprises Hg(OAc)₂.

In still another embodiment, the invention provides a method, wherein the phosphorus-containing compound of formula VIII-a is triethyl phosphonoacetate and the base is lithium hexamethyldisalazide (LiHMDS).

In another embodiment, the invention provides a method, wherein the organometallic reagent is ethyl magnesium bromide (EtMgBr).

In a further embodiment, the invention provides a method, wherein the conversion takes place at a reaction temperature of about 120° C.

In another further embodiment, the invention provides a method, further comprising the addition of cerium trichloride (CeCl₃).

In one embodiment, the invention provides a method, wherein the compound of formula VI is Acetic acid 1-ethylidene-7a-methyl-octahydro-inden-4-yl ester:

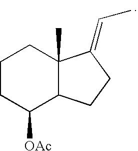

In another embodiment, the invention provides a method, wherein the compound of formula VII is Acetic acid 1-ethylidene-2-hydroxy-7a-methyl-octahydro-inden-4-yl ester:

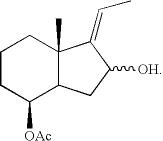

In yet another embodiment, the invention provides a method, wherein the compound of formula VIII is Acetic acid 7a-methyl-1-(1-methyl-3-oxo-propyl)-3a,4,5,6,7,7a-hexahydro-3H-inden-4-yl ester:

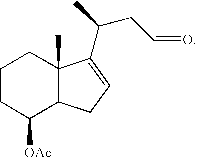

In still another embodiment, the invention provides a method, wherein the compound of formula IX is 5-(4-Acetoxy-7a-methyl-3a,4,5,6,7,7a-hexahydro-3H-inden-1-yl)-hex-2-enoic acid ethyl ester:

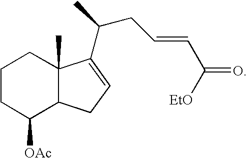

In another embodiment, the invention provides a method, wherein the compound of formula X is 1-(5-Ethyl-5-hydroxy-1-methyl-hept-3-enyl)-7a-methyl-3a,4,5,6,7,7a-hexahydro-3H-inden-4-ol:

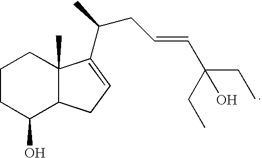

In another embodiment, the invention provides a method of producing a vitamin D₃ compound of formula I, further comprising obtaining a compound of formula VI. In a one embodiment, the compound of formula VI is obtained by synthesis by a method comprising:

converting compound 3

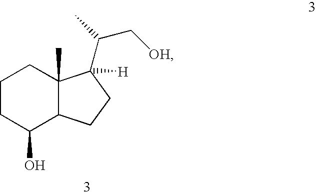

to compound 14

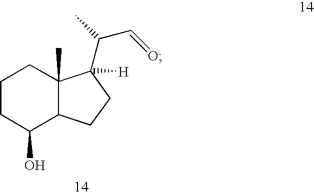

converting compound 14 to compound of formula XX

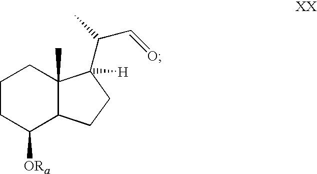

wherein Rₐ is a hydroxy protecting group; and converting compound of formula XX to a compound of formula VI. In one embodiment, the oxidation reagent for the conversion of 3 to 14 comprises TEMPO, tetrabutylammonium chloride hydrate and N-chlorosuccinimide. In still another embodiment, the invention provides a method wherein the compound of formula XX is Acetic acid 7a-methyl-1-(1-methyl-2-oxo-ethyl)-octahydro-inden-4-yl ester:

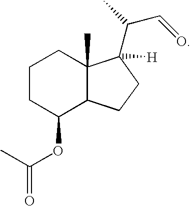

In another embodiment, the compound of formula VI is obtained by synthesis by a method comprising:

converting compound 3

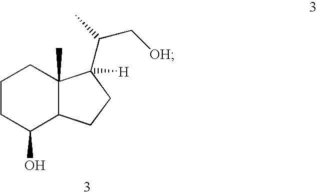

to a compound of formula XXI

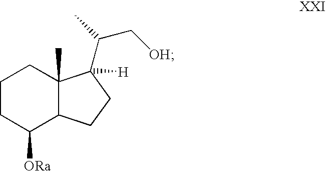

wherein Rₐ is a hydroxy protecting group;

converting a compound of formula XXI to compound of formula XX

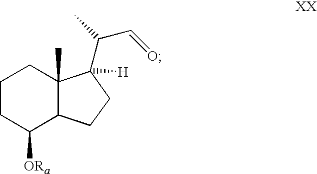

wherein Rₐ is a hydroxy protecting group; and converting compound of formula XX to a compound of formula VI. In certain embodiments, the oxidation reagent for the conversion of XXI to XX comprises oxalyl chloride. In another embodiment, the invention provides a method wherein the compound of formula XXI is Acetic acid 1-(2-hydroxy-1-methyl-ethyl)-7a-methyl-octahydro-inden-4-yl ester:

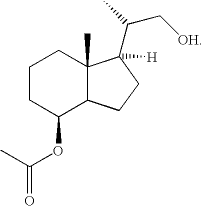

In one aspect, the invention provides a method of producing a vitamin D₃ compound of formula I:

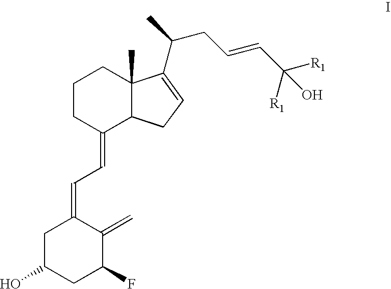

wherein each R₁ is independently alkyl; and pharmaceutically acceptable esters, salts, and prodrugs thereof; which comprises converting a compound of formula XII

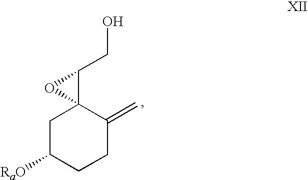

wherein Rₐ is a hydroxy protecting group, to a compound of formula XII-a

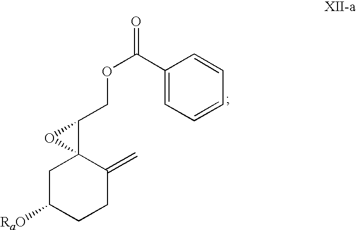

converting the compound of formula XII-a to a compound of formula XV

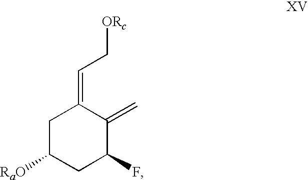

wherein R_{c} is H or benzoyl; converting the compound of formula XV to a compound of formula III

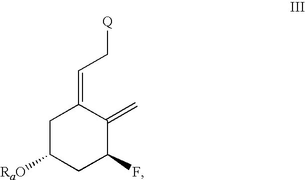

wherein Q is a phosphorus-containing group; and reacting the compound of formula III with a compound of formula II

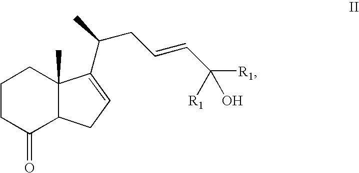

to thereby produce a compound of formula I.

In another aspect, the invention provides a method of producing a compound of formula XV

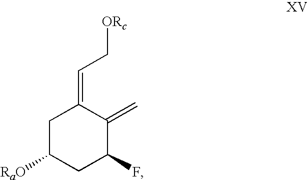

wherein R_{c} is H or benzoyl; which comprises converting a compound of formula XII

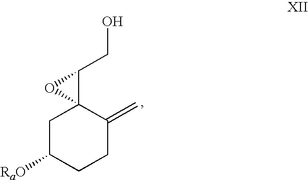

to a compound of formula XII-a

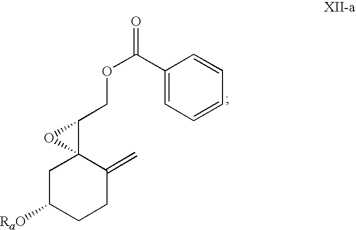

and

converting the compound of formula XII-a to a compound of formula XV, to thereby produce a compound of formula XV.

In one embodiment, the invention provides a method, wherein the conversion of the compound of formula XII to the compound of formula XII-a is carried out in the presence of benzoyl chloride and base.

In another embodiment, the invention provides a method, further comprising reacting the compound of formula XII-a

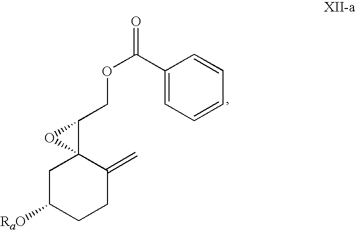

with an oxidizing agent, to provide a compound of formula XIII

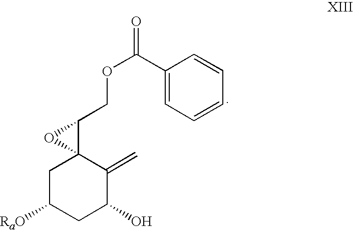

In still another embodiment, the invention provides a method, further comprising reacting the compound of formula XIII

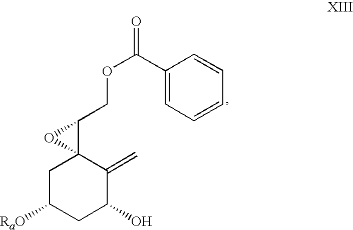

with a fluorinating agent, to provide a compound of formula XIV

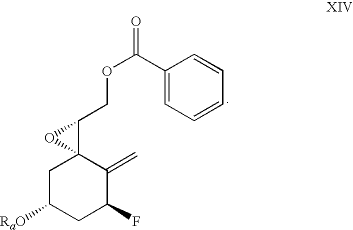

In yet another embodiment, the invention provides a method, further comprising reacting the compound of formula XIV

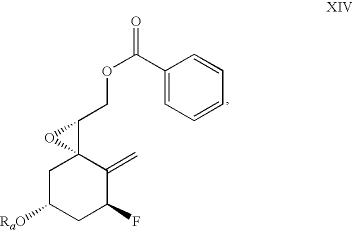

with a deoxygenation agent, to provide a compound of formula XV

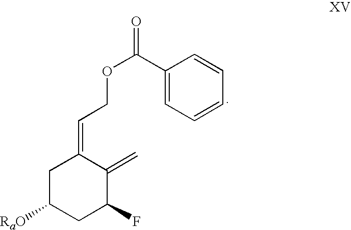

In another embodiment, the invention provides a method, further comprising reacting the compound of formula XV

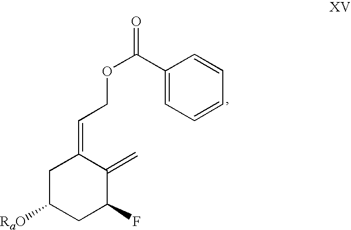

with a deprotection agent, to provide a compound of formula XV

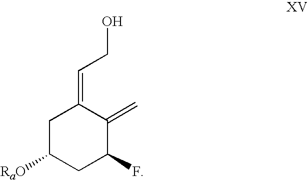

In another embodiment, the invention provides a method, further comprising: reacting the compound of formula XIV

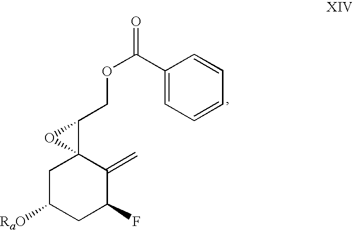

with a deoxygenation agent, to provide a compound of formula XVa

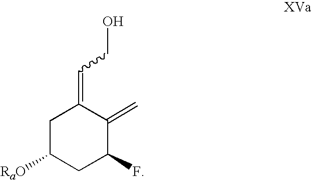

In another embodiment, the invention provides a method, further comprising: reacting the compound of formula XVa

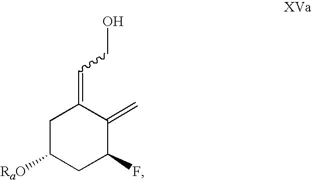

with an epimerizing agent, to provide a compound of formula XV

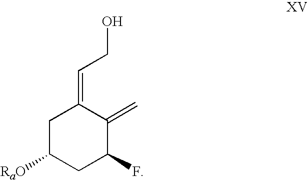

In still another embodiment, the invention provides a method, further comprising reacting the compound of formula XV

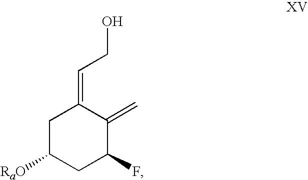

with a chlorinating agent, to provide a compound of formula XVI

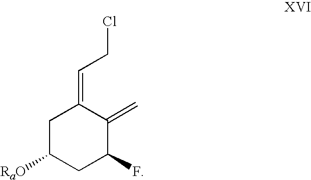

In another embodiment, the invention provides a method, further comprising reacting the compound of formula XVI

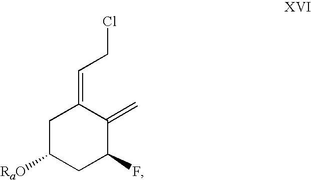

with a phosphorous containing agent in the presence of a base, to provide a compound of formula III

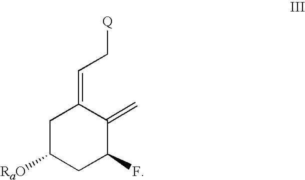

In a further embodiment, the invention provides a method, wherein the base is pyridine.

In one embodiment, the invention provides a method, wherein the oxidizing reagent comprises selenium dioxide and t-butyl hydrogen peroxide.

In another embodiment, the invention provides a method, wherein the fluorinating agent is diethylaminosulfur trifluoride (DAST).

In yet another embodiment, the invention provides a method, wherein the deoxygenation reagent is tris(3,5-dimethylpyrazoyl)hydridoborate rhenium trioxide or tungsten hexachloride/nBuLi.

In still another embodiment, the invention provides a method, wherein the deprotection agent is sodium methoxide.

In yet another embodiment, the invention provides a method, wherein the epimerization agent is hv and 9-fluorenone.

In another embodiment, the invention provides a method, wherein the chlorinating agent comprises triphosgene and pyridine.

In yet another embodiment, the invention provides a method, wherein the phosphorous containing agent is diphenyl phosphine oxide.

In another further embodiment, the invention provides a method, wherein the base is sodium hydride.

In one embodiment, the invention provides a method, wherein the compound of formula XII-a is Benzoic acid 7-(tert-butyl-dimethyl-silanyloxy)-4-methylene-1-oxa-spiro[2.5]oct-2-ylmethyl ester:

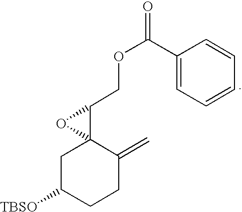

In another embodiment, the invention provides a method, wherein the compound of formula XIII is Benzoic acid 7-(tert-butyl-dimethyl-silanyloxy)-5-hydroxy-4-methylene-1-oxa-spiro[2.5]oct-2-ylmethyl ester:

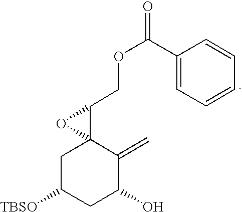

In yet another embodiment, the invention provides a method, wherein the compound of formula XIV is Benzoic acid 7-(tert-butyl-dimethyl-silanyloxy)-5-fluoro-4-methylene-1-oxa-spiro[2.5]oct-2-ylmethyl ester:

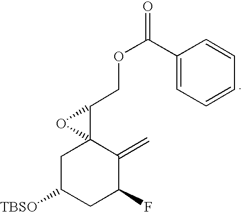

In still another embodiment, the invention provides a method, wherein the compound of formula XV is Benzoic acid 2-[5-(tert-butyl-dimethyl-silanyloxy)-3-fluoro-2-methylene-cyclohexylidene]-ethyl ester:

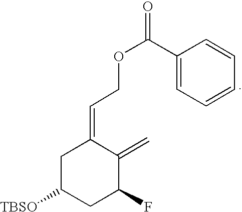

In another embodiment, the invention provides a method, wherein the compound of formula XV is 2-[5-(tert-Butyl-dimethyl-silanyloxy)-3-fluoro-2-methylene-cyclohexylidene]-ethanol:

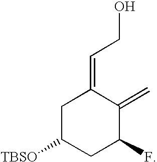

In another embodiment, the invention provides a method, wherein the compound of formula XVa is 2-[5-(tert-Butyl-dimethyl-silanyloxy)-3-fluoro-2-methylene-cyclohexylidene]-ethanol:

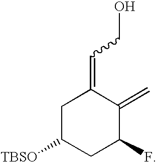

In still another embodiment, the invention provides a method, wherein the compound of formula XVI is tert-Butyl-[3-(2-chloro-ethylidene)-5-fluoro-4-methylene-cyclohexyloxy]-dimethyl-silane:

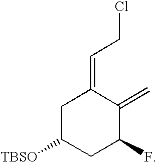

In yet another embodiment, the invention provides a method, wherein the compound of formula III is tert-Butyl-{3-[2-(diphenyl-phosphinoyl)-ethylidene]-5-fluoro-4-methylene-cyclohexyloxy}-dimethyl-silane:

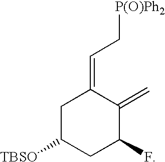

In one embodiment, the invention provides a method, wherein the coupling reaction of the compound of formula II and the compound of formula III to form the compound of formula I comprises converting the compound of formula II

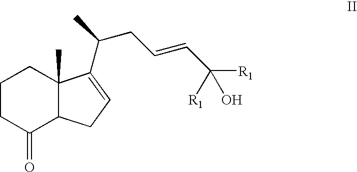

to a compound of formula XVII

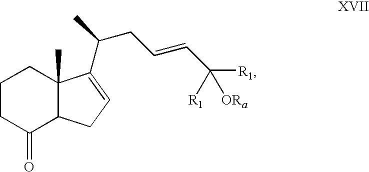

wherein Rₐ is hydroxy protecting group; reacting the compound of formula XVII with a compound of formula III in the presence of base

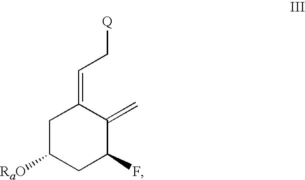

wherein Q is a phosphorus-containing group, to form a compound of formula XVIII

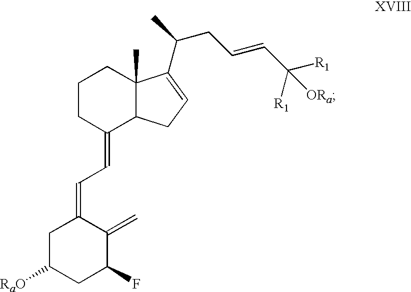

and

converting the compound of formula XVIII to the compound of formula I.

In another embodiment, the invention provides a method, wherein the reaction of the compound of formula II and the compound of formula III to produce the compound of formula I is carried out in a single process step.

In still another embodiment, the invention provides a method, wherein the compound of formula I is produced in 21 process steps.

In yet another embodiment, the invention provides a method, wherein the compound of formula I is produced in 19 process steps.

In one embodiment, the invention provides the methods described herein, wherein each R₁ is ethyl in the compound of formula I.

In another aspect, the invention provides a method for producing compounds of formula I by reacting a compound of formula II

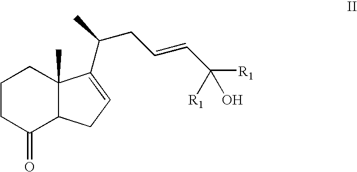

with a compound of formula III

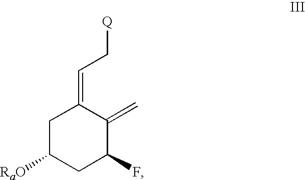

wherein Rₐ is defined as above and Q is a phosphorus-containing group in the presence of a strong base. This coupling reaction has the advantage of not requiring protecting the hydroxyl group on the compound of formula II, thereby eliminating a subsequent deprotection step.

In a preferred embodiment, the invention provides a method for producing 1α-Fluoro-25-hydroxy-16-23E-diene-26,27-bishomo-20-epi-cholecalciferol (1):

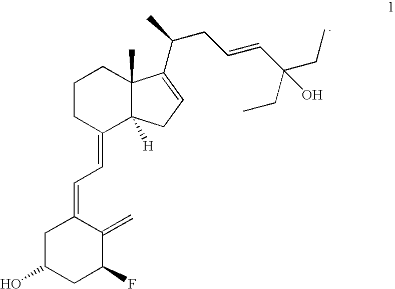

In one embodiment, the invention provides a method in which the total synthesis is of compound 1 is carried out in 21 steps. In another embodiment, the invention provides a method in which the total synthesis is of compound 1 is carried out in 19 steps.

In certain embodiments, the method includes the step of obtaining compound 3. In one embodiment, compound 3 is obtained by synthesis by a method comprising:

converting compound 2

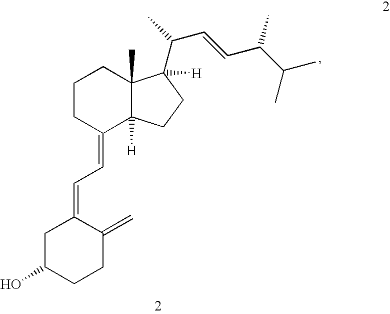

to compound 7

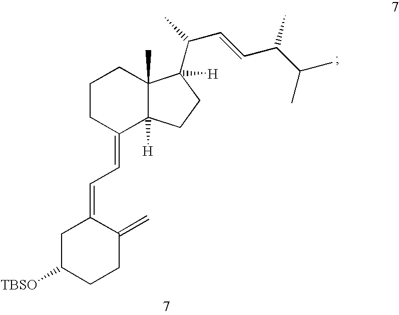

and converting compound 7 to compound 3.

In other embodiments, the method includes the step of obtaining the compound of formula XII. In one embodiment, the compound of formula XII is obtained by synthesis by a method comprising:

converting compound 2

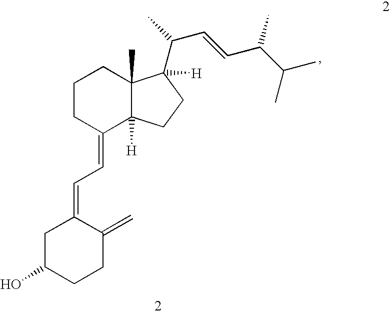

to compound 4a

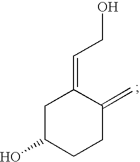

converting compound 4a to compound 4

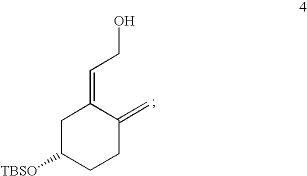

and converting compound 4 to a compound of formula XII. In certain embodiments, the epoxidation reagent is m-chloroperoxybenzoic acid (M-CPBA).

In carrying out the methods of the invention, a number of reagents and reaction conditions can be used. Although the following is a description of certain preferred reagents and reaction conditions, one of ordinary skill in the art will readily appreciate that reagents and reaction conditions can be varied without undue experimentation and without departing from the spirit of the invention.

Oxidizing agents known in the art include, but are not limited to SeO₂/t-BuOOH, Jones reagent (H₂CrO₄, CrO₃), VO(acac)₂/tBuOOH, dipyridine Cr(VI) oxide, pyridinium chlorochromate, pyridinium dichromate (PDC), sodium hypochlorite/acetic acid NaOCl/HOAc), Cl₂-pyridine, hydrogen peroxide/ammonium molybdate, NaBrO₃/CAN, KMnO₄, Br₂, MnO₂, NBS/tetrabutylammonium iodide, ruthenium tetroxide, mCPBA, TEMPO/NCS. Preferably, the oxidizing agents of the present invention are SeO₂/t-BuOOH, mCPBA, TEMPO/NCS, and PDC.

Oxidation reaction times range from 0.5 h to 72 h. In certain embodiments, the TEMPO/NCS oxidation was carried out over 24-48 h, preferably 24-38 h. In certain embodiments, the SeO₂/t-BuOOH oxidation was carried out over 24-72 h, preferably 72 h. In other embodiment, the SeO₂/t-BuOOH oxidation was carried out over 24-36 h, preferably 36 h. Typical reaction conditions include high temperatures of from about 0° C. to about 150° C. Preferred temperatures include a range of from about 25° C. to about 150° C.

Decarbonylation reagents include combinations of metal catalysts and ligands. Metal catalysts include, but are not limited to Rh/C, Ru/C, Pd(OAc)₂, Pd(PPh₃)₄, Rh(PPh₃)₃Cl, Al₂O₃, and Pd/C. Other catalyst/ligand systems include Rh₂(OAc)₄/N₂C(CO₂Me)₂, and tris(3,5-dimethylpyrazoyl)hydridoborate rhenium trioxide/triphenylphosphine. Ligands include but are not limited to dibenzylideneacetone (dba) and benzylideneacetone. High reaction temperatures provided the desired product in high yields with reduced byproduct formation. Temperatures for decarbonylation reactions range from about 25° C. to about 250° C., preferably about 100° C. to about 250° C., preferably about 100° C. or 230° C.

Preferred reactants utilized in Claisen rearrangements include Hg(OAc)₂ and ethyl vinyl ether or [Ir(COD)Cl]₂ and vinyl acetate. (COD is cyclooctadiene) Typical reaction conditions include high temperatures of from about 25° C. to about 150° C. Preferred temperatures include a range of from about 50° C. to about 150° C., preferably about 100° C. or preferably about 120° C. Reaction times are substrate dependent. Claisen rearrangements were allowed to run for 1 h-48 h. In certain embodiments, the Claisen rearrangements were allowed to run for 12 h-24 h, preferably 24 h.

Phosphorous containing reagents are phosphorous containing compounds utilized to form compounds used in coupling reactions with carbonyl functionalities to provide compounds with alkene and alkyne groups, e.g. Wittig-type reactions. Typical phosphorous containing reagents used to make Wittig-type reagents include, but are not limited to, triphenylphosphine, trialkylphosphine, diphenylphosphine oxide, and triethyl phosphonoacetate.

Wittig-type reactions are carried out in the presence of a phosphorus-containing compound and carbonyl compound. The present invention provides for the formation of E-double bonds, which are selectively produced from a combination of Wittig reagent, base, and reaction temperature. It is preferred that (EtO)₂POCH₂COOEt is the phosphorous agent, lithium hexamethyl disalazide (LiHMDS) is the base, and the reaction is carried out at a temperature of about −100° C. to about 0° C., preferably about −85° C. to about −78° C.

The 1,2 reduction of unsaturated esters is carried out in the presence of organometallic reagents mediated by Lewis acids. Organometallic reagents include but are not limited to Grignard reagents and organolithium reagents such as ethyl magnesium bromide and ethyl lithium. Lewis acids utilized in this reduction include, but are not limited to CeCl₃, Al(Oi-Pr)₃, AlCl₃, TiCl₄, BF₃, SnCl₄, and FeCl₃, preferably CeCl₃. In certain embodiments, CeCl₃ was dried in vacuo prior to use.

Benzoyl group deprotection agents known in the art include, but are not limited to sodium methoxide, triethyl amine/water/methanol, potassium cyanide, boron trifluoride/etherate/dimethyl sulfide, and electrolytic cleavage. Preferably, the benzoyl group deprotection agent of the invention is sodium methoxide.

Chlorinating reagents known in the art include, but are not limited to hydrochloric acid (HCl), thionyl chloride (SOCl₂), tosylchloride and lithium chloride; and triphosgene and pyridine. Preferably, triphosgene and pyridine is utilized.

### 4. Novel Intermediates

The methods of the invention involve the generation and use of certain novel intermediate compounds. Novel intermediates of the invention include the following compounds:

### Acetic acid 1-ethylidene-2-hydroxy-7a-methyl-octahydro-inden-4-yl ester

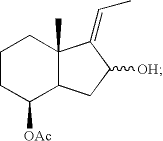

### Acetic acid 7a-methyl-1-(1-methyl-3-oxo-propyl)-3a,4,5,6,7,7a-hexahydro-3H-inden-4-yl ester

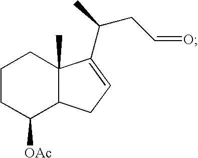

### 5-(4-Acetoxy-7a-methyl-3a,4,5,6,7,7a-hexahydro-3H-inden-1-yl)-hex-2-enoic acid ethyl ester

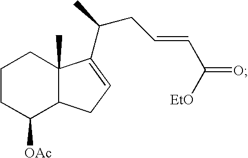

### Benzoic acid 7-(tert-butyl-dimethyl-silanyloxy)-5-fluoro-4-methylene-1-oxa-spiro[2.5]oct-2-ylmethyl ester:

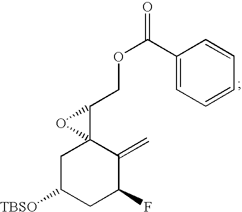

### Benzoic acid 2-[5-(tert-butyl-dimethyl-silanyloxy)-3-fluoro-2-methylene-cyclohexylidene]-ethyl ester

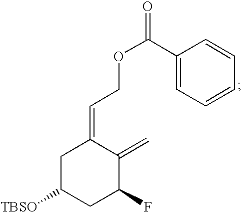

### Acetic acid 1-(2-hydroxy-1-methyl-ethyl)-7a-methyl-octahydro-inden-4-yl ester

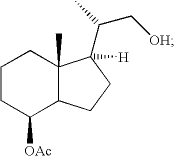

and

### 4-(tert-Butyl-dimethyl-silanyloxy)-2-[2-(tert-butyl-dimethyl-silanyloxy)-ethylidene]-1-methylene-cyclohexane

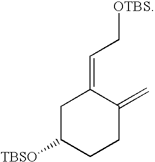

### EXEMPLIFICATION OF THE INVENTION

The invention is further illustrated by the following examples which should in no way should be construed as being further limiting.

### Synthesis of Compounds of the Invention

### Experimental

All operations involving vitamin D₃ analogs were conducted in amber-colored glassware in a nitrogen atmosphere. Tetrahydrofuran was distilled from sodium-benzophenone ketyl just prior to its use and solutions of solutes were dried with sodium sulfate. Melting points were determined on a Thomas-Hoover capillary apparatus and are uncorrected. Optical rotations were measured at 25° C. ¹H NMR spectra were recorded at 400 MHz in CDCl₃ unless indicated otherwise. TLC was carried out on silica gel plates (Merck PF-254) with visualization under short-wavelength UV light or by spraying the plates with 10% phosphomolybdic acid in methanol followed by heating. Flash chromatography was carried out on 40-65 μm mesh silica gel. Preparative HPLC was performed on a 5×50 cm column and 15-30 μm mesh silica gel at a flow rate of 100 mL/min.

### Example 1

### Cleavage of the Vitamin D2 Starting Material

### t-Butyl-dimethyl-(4-methylene-3-{2-[7a-methyl-1-(1,4,5-trimethyl-hex-2-enyl)-octahydro-inden-4-ylidene]-ethylidene}-cyclohexyloxy)-silane (7)

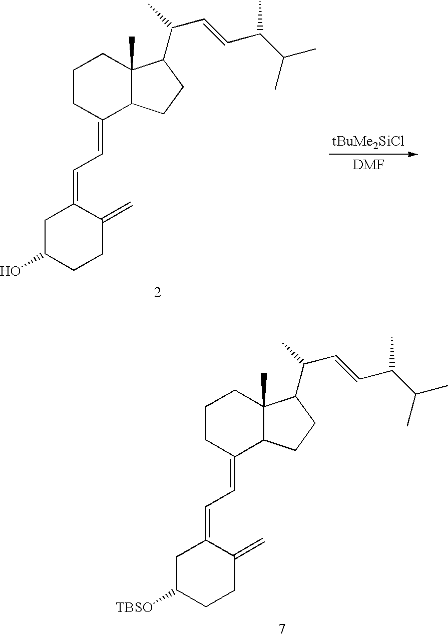

To a stirred solution of 2 (100.00 g, 0.25 mol) in DMF (250 mL), imidazole (40.80 g, 0.6 mol) and (t-butyldimethyl)silyl chloride (45.40 g, 0.3 mol) were added successively. The reaction mixture was stirred at room temperature for 1 h, diluted with hexane (750 mL), washed with water (500 mL), 1N HCl (500 mL), brine (500 mL) and dried over Na₂SO₄. The residue (155 g) after evaporation of the solvent was filtered through a plug of silica gel (500 g, 5% AcOEt in hexane) to give the title compound (115.98 g, 0.23 mol, 92%). ¹H-NMR: δ 0.04 and 0.08 (2s, 6H), 0.59 (s, 3H), 0.90 (d, 3H, J=6.6 Hz), 0.92 (d, 3H, J=6.6 Hz), 0.98 (s, 9H), 0.99 (d, 3H, J=7.0 Hz), 1.06 (d, 3H, J=6.8 Hz), 1.10-2.95 (m, 21H), 5.11 (br s, 2H), 5.22 (m, 2H), 6.49 (br s, 2H).

### 2-[5-(tert-Butyl-dimethyl-silanyloxy)-2-methylene-cyclohexylidene]-ethanol (4) and 1-(2-Hydroxy-1-methyl-ethyl)-7a-methyl-octahydro-inden-4-ol (3)

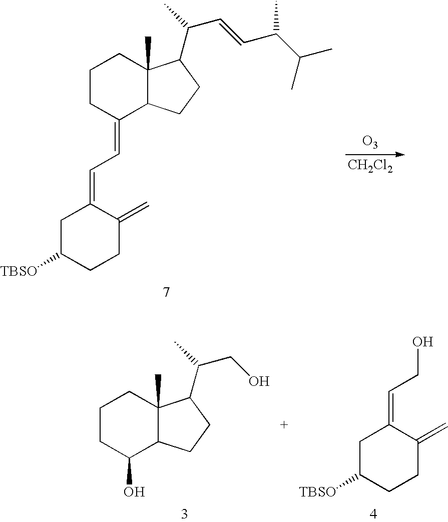

A stream of ozone was passed through a stirred solution of 7 (23.4 g, 45.8 mmol), pyridine (5.0 mL) and Sudane Red 7B (15.0 mg) in dichloromethane (550 mL), at −55 to −60° C. until Sudane Red decolorized (55 min). Sodium borohydride (6.75 g, 180 mmol) was then added followed by ethanol (250 mL). The reaction was allowed to warm to room temperature and stirred at room temperature for 1 h. Acetone (15 mL) was added and, after 30 min brine (300 mL) was added. The mixture was diluted with ethyl acetate (500 mL) and washed with water (600 mL). The aqueous phase was extracted with AcOEt (300 mL). The combined organic phases were dried over Na₂SO₄. The residue (26.5 g), after evaporation of the solvent, was filtered through a plug of silica gel (500 g, 15%, 30% and 50% AcOEt in hexane) to give: Fraction A (5.9 g, mixture containing the desired A-ring (ca 83% pure by NMR) ¹H NMR: δ 5.38 (1H, t, J=6.4 Hz), 4.90 (1H, brs), 4.57 (1H, brs), 4.22 (1H, dd, J=7.3, 12.5 Hz), 4.13 (1H, dd, J=6.3, 12.5 Hz), 3.78 (1H, m), 2.40-1.30 (6H, m), 0.83 (9H, s), 0.01 (3H, s), 0.00 (3H, s); Fraction A was used for the synthesis of the A-ring precursor. Fraction B (14.6 g, mixture containing a CD-rings fragments on a different stage of oxidation). Fraction B was further ozonolyzed in order to obtain the Lythgoe diol (3). A stream of ozone was passed through a stirred solution of Fraction B (14.6 g) and Sudane Red 7B (3.0 mg) in ethanol (225 mL) at −55 to −60° C. for 30 min (Sudane Red decolorized). Sodium borohydride (3.75 g, 100 mmol) was added and the reaction was allowed to warm to room temperature and stirred at room temperature for 1 h. Acetone (5 mL) was added and, after 30 min brine (200 mL) was added. The mixture was diluted with dichloromethane (300 mL) and washed with water (250 mL). The aqueous phase was extracted with dichloromethane (200 mL). The combined organic phases were, evaporated to dryness (the last portion was evaporated with addition of toluene 100 mL). The residue (16.2 g) was dissolved in dichloromethane (100 mL), concentrated to a volume of ca 20 mL diluted with petroleum ether (30 mL) and set aside in the fridge for crystallization. The white powder was filtered of (4.05 g), the mother liquor was concentrated and filtered through silica gel (100 g, 5% MeOH in CH₂Cl₂) to give yellow oil (9.4 g), which was recrystallized (20 mL, dichloromethane; petroleum ether 1:2) to give white powder (1.79 g). Thus the total yield of the Lythgoe diol 3 was (5.84 g, 27.5 mmol, 60% from D₂) ¹H NMR: δ 4.08 (1H, m), 3.64 (1H, dd, J=3.3, 10.6 Hz), 3.39 (1H, dd, J=6.6, 10.6 Hz), 2.04-1.14 (15H, m), 1.03 (3H, d, J=6.6 Hz), 0.96 (3H, s).

### 1-(2-Hydroxy-1-methyl-ethyl)-7a-methyl-octahydro-inden-4-ol (4a) and 1-(2-Hydroxy-1-methyl-ethyl)-7a-methyl-octahydro-inden-4-ol (3)

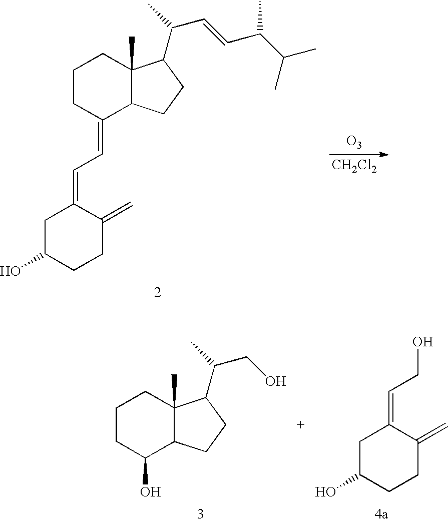

Compound 2 (98.8 g, 249 mmol) was dissolved in dichloromethane (900 mL) and ethanol (400 mL), pyridine (25.0 mL) and Sudane Red 7B (30.0 mg) were added and the mixture was cooled down to −65 to −70° C. A stream of ozone was passed through for 3 h. (until Sudane Red decolorized, reaction was also followed by TLC and decolorization of Sudane Red corresponds to consumption of Vitamin D₂). Sodium borohydride (24.0 g, 0.64 mol) was added portion-wise and the reaction was allowed to warm to room temperature and stirred at room temperature for 1 h. Acetone (75 mL) was added portion-wise (to keep temperature under 35° C.) and the reaction mixture was stored overnight in the fridge. The mixture was washed with water (600 mL). The aqueous phase was extracted with dichloromethane (6×300 mL). The combined organic phases were dried over Na₂SO₄ The residue (118 g) after evaporation of the solvent was passed through a plug of silica gel (0.5 kg, 30%, 50% AcOEt in hexane) to give: Fraction A (69.7 g, CD-rings figments); Fraction B (4.8 g of a pure Lythgoe diol 3 after crystallization from hexane:AcOEt 3:1); Fraction C (12.3 g of a pure compound 2, after crystallization from AcOEt); Fraction D (11.5 g, mixture of the desired compound 2 and 4-Methylene-cyclohexane-1,3-diol).

Fraction A was further ozonolyzed in order to obtain (3). A stream of ozone was passed through a stirred solution of Fraction A (69.7 g) in ethanol (500 mL), dichloromethane (600 mL) and Sudane Red 7B (3.0 mg) at −65 to −70° C. for 3 h. (Sudane Red decolorized). Sodium borohydride (22.5 g, 0.6 mol) was added and the reaction was allowed to warm to room temperature and stirred at room temperature for 1 h. Acetone (125 mL) was added portion-wise (to keep temperature under 35° C.) and the reaction mixture was stored overnight in the fridge. The mixture was washed with water (600 mL). The aqueous phase was extracted with dichloromethane (2×300 mL) and with AcOEt (300 mL). The combined organic phases were dried over Na₂SO₄ and evaporated to dryness. The residue (55.0 g) was purified by crystallization (AcOEt:Hexane 1:2) to give: Fraction E (15.7 g of a pure crystalline 3); Fraction F (35 g, of mixture containing Lythgoe diol 3). Fraction F (35 g) was passed through a plug of silica gel (0.5 kg, 30%, 50% AcOEt in hexane) to give after crystallization (AcOEt:Hexane 1:2) Fraction G (18.9 g), thus the overall yield of (3) was 39.4 g 74.5% from 2).

¹H NMR: δ 5.38 (1H, t, J=6.4 Hz), 4.90 (1H, brs), 4.57 (1H, brs), 4.22 (1H, dd, J=7.3, 12.5 Hz), 4.13 (1H, dd, J=6.3, 12.5 Hz), 3.78 (1H, m), 2.40-1.30 (6H, m), 0.83 (9H, s), 0.01 (3H, s), 0.00 (3H, s).

Fraction D (11.5 g) was passed through a plug of silica gel (0.3 kg, 50% AcOEt in hexane) to give (after crystallization (AcOEt): Fraction H (1.1 g of a pure crystalline compound 4a, 2.8%); Fraction I (10.2 g, mixture of the desired compound 4a. Thus the overall yield of the isolated (S)-(Z)-3-(2-Hydroxy-ethylidene)-4-methylene-cyclohexanol (4a) is 13.4 g, 34.8%

¹H NMR: δ 5.51 (1H, t, J=6.6 Hz), 5.03 (1H, brs), 4.66 (1H, brs), 4.24 (2H, m), 3.94 (1H, m), 2.55 (1H, dd, J=3.9, 13.2 Hz), 2.41 (1H, m), 2.25 (1H, dd, J=7.8, 12.9 Hz), 1.94 (1H, m), 1.65 (1H, m).

### (S)-(Z)-2-[5-(tert-butyldimethyl)silanyloxy)-2-methylene-cyclohexylidene]-ethanol (4)

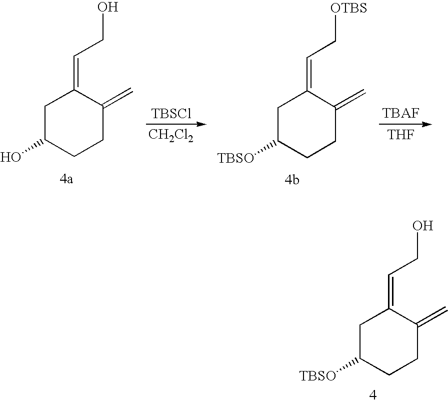

To a stirred solution (S)-(Z)-3-(2-Hydroxy-ethylidene)-4-methylene-cyclohexanol (4a) (4.04 g, 26.3 mmol) in dichloromethane (40 mL), imidazole (5.36 g, 78.7 mmol) and (tert-butyldimethyl)silyl chloride (9.50 g, 63.0 mmol) were added successively. The reaction mixture was stirred at room temperature for 100 min. after which water (25 mL) was added. After 15 min. the mixture was diluted with hexane (350 mL), washed with water (2×100 mL) and brine (50 mL) and dried over Na₂SO₄. The residue (10.7 g) after evaporation of the solvent was dissolved in tetrahydrofurane (50 mL), Bu₄NF (26.5 mL, 1M/THF) was added at +5° C. and the mixture was stirred at +5° C. for 45 min. and additional 30 min. at room temperature. The mixture was diluted with water (100 mL) and ethyl acetate (250 mL). After separation organic layer was washed with water (100 mL) and brine (50 mL). Aqueous layers were extracted with ethyl acetate (5×50 mL). The combined organic layers were dried over Na₂SO₄. The residue after evaporation of the solvent was purified by FC (150 g, 10%, 50% and 100% AcOEt in hexane) to give the titled compound 4. (6.43 g, 85% pure by NMR, 78% of the title compound,)

¹H NMR: δ 5.38 (1H, t, J=6.4 Hz), 4.90 (1H, brs), 4.57 (1H, brs), 4.22 (1H, dd, J=7.3, 12.5 Hz), 4.13 (1H, dd, J=6.3, 12.5 Hz), 3.78 (1H, m), 2.40-1.30 (6H, m), 0.83 (9H, s), 0.01 (3H, s), 0.00 (3H, s).

### Example 2

### 1. Synthesis of the A-Ring Precursor

### (2R,3S,7S)-[7-(t-butyldimethyl)silanyloxy)-4-methylene-1-oxa-spiro[2.5]oct-2-yl]-methanol (8)

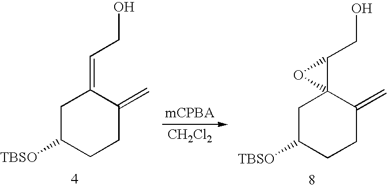

To a stirred solution of a crude 4 (5.9 g, ca 18.3 mmol, Fraction A from ozonolysis) in dichloro-methane (120 mL) at room temperature, AcONa (2.14 g, 26.1 mmol) was added followed by 72% mCPBA (4.32 g, 18.0 mmol). The reaction mixture was then stirred at 10° C. for ½ h then diluted with hexane (200 mL) washed with 10% K₂CO₃ (3×150 mL), and dried over Na₂SO₄. The residue after evaporation of solvent (6.6 g) was filtered through a plug of silica gel (150 g, 10% AcOEt in hexane) to give the crude title compound (4.87 g, ca 15.4 mmol, 84%) ¹H-NMR: δ 0.063 and 0.068 (2s, 6H), 0.88 (s, 9H), 1.38-1.49 (m, 1H), 1.54 (m, 1H, OH), 1.62 (m, 1H), 1.96 (m, 3H), 2.43 (m, 1H), 3.095 (t, 1H, J=5.6 Hz), 3.60 (m, 2H), 3.86 (m, 1H), 4.91 (m, 1H).

### Benzoic acid (2R,3S,7S)-7-(t-butyldimethyl)silanyloxy)-4-methylene-1-oxa-spiro[2.5]oct-2-yl methyl ester (9)

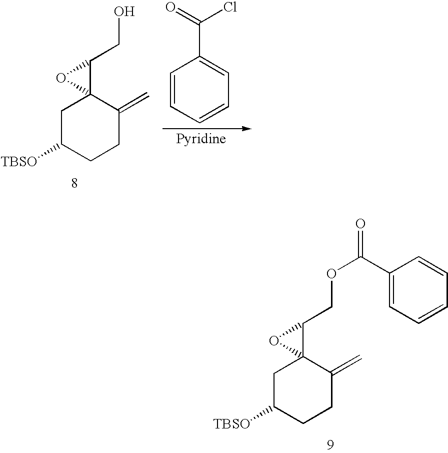

To a stirred solution of 8 (4.87 g, ca 15.4 mmol) in pyridine (25 mL) at room temperature, benzoyl chloride (2.14 mL, 18.4 mmol) was added and the reaction mixture was stirred for 1 h. Water (25 mL) was added and after stirring for 45 min at room temperature the mixture was diluted with hexane (80 mL), washed with saturated NaHCO₃ solution (50 mL), and dried over Na₂SO₄. The residue after evaporation of solvent (17.5 g) was purified by FC (150 g, 10% AcOEt in hexane) to give the title compound (5.44 g, 14.0 mmol, 91%) ¹H NMR: δ 8.04-7.80 (2H, m), 7.56-7.50 (1H, m), 7.44-7.37 (2H, m), 4.94 (1H, brs), 4.92 (1H, brs), 4.32 (1H, dd, J=4.8, 11.9 Hz), 4.14 (1H, dd, J=6.2, 11.9 Hz), 3.83 (1H, m), 3.21 (1H, dd, J=4.8, 6.2 Hz), 2.42 (1H, m), 2.04-1.90 (3H, m), 1.64-1.34 (2H, m), 0.83 (9H, s), 0.02 (3H, s), 0.01 (3H, s).

### Benzoic acid (2R,3S,5R,7S)-7-(t-butyldimethyl)silanyloxy)-5-hydroxy-4-methylene-1-oxa-spiro[2.5]oct-2-yl methyl ester (10)

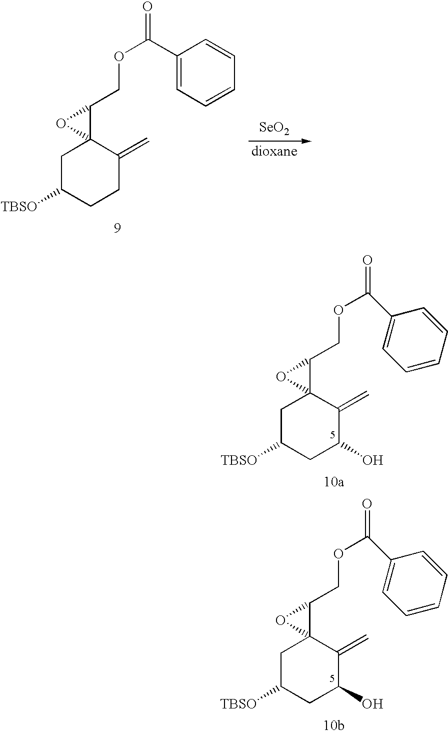

To a stirred solution of 9 (10.0 g, 25.7 mmol)) in dioxane (550 mL) at 85° C. was added selenium dioxide, (3.33 g, 30.0 mmol) followed by t-butyl hydrogen peroxide (9.0 mL, 45.0 mmol, 5-6 M in nonane) and the reaction mixture was stirred at 85° C. for 16 h, after which selenium dioxide (1.11 g, 10.0 mmol) was added followed by t-butyl hydrogen peroxide (3.0 mL, 15.0 mmol, 5-6 M in nonane) and the reaction mixture was stirred at 85° C. for additional 6 h. The solvent was removed under vacuum and the residue (15.3 g) was filtered through a plug of silica gel (300 g, 20% AcOEt in hexane) to give: starting material (970 mg, 10%) and a mixture of 10a and 10b (8.7 g). This mixture was divided into 3 portion (2.9 g each) and purified twice by FC (200 g, 5% isopropanol in hexane, same column was used for all six chromatographs) to give: 10b (1.83 g, as a 10:1 mixture of 10b:10a ca 16% of 5α-hydroxy compound); 10a (6.0 g, 14.8 mmol, 58%) as white crystals. The structure of 10a was confirmed by X-ray crystallography. ¹H NMR: δ 8.02-7.90 (2H, m), 7.58-7.50 (1H, m), 7.46-7.38 (2H, m), 5.25 (1H, br s), 5.11 (1H, br s), 4.26 (1H, dd, J=5.5, 12.1 Hz), 4.15 (1H, dd, J=5.9, 12.1 Hz), 4.07 (1H, m), 3.87 (1H, m), 3.19 (1H, dd, J=5.5, 5.9 Hz), 2.34-1.10 (5H, m), 0.81 (9H, s), 0.01 (3H, s), 0.00 (3H, s).

### Benzoic acid (2R,3S,5S,7R)-7-(t-butyldimethyl)silanyloxy)-5-fluoro-4-methylene-1-oxa-spiro[2.5]oct-2-ylmethyl ester (11)

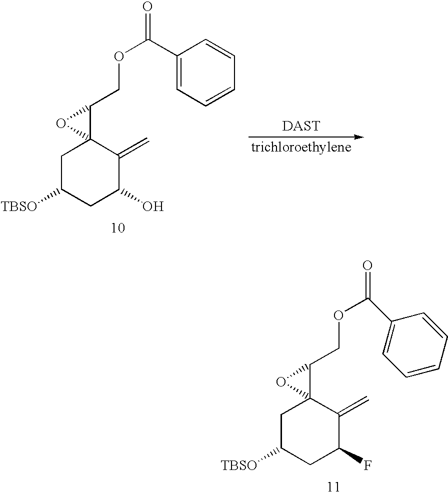

To a stirred solution of a diethylaminosulfur trifluoride (DAST) (2.0 mL, 16.0 mmol) in trichloroethylene (20 mL) a solution of 10 (2.78 g, 6.87 mmol) in trichloroethylene (126 mL was added at −75° C. After stirring for 20 min at −75° C. methanol (5.5 mL) was added followed by saturated NaHCO₃ solution (6 mL) and the resulting mixture was diluted with hexane (150 mL) and washed with saturated NaHCO₃ solution (100 mL), dried over Na₂SO₄ and concentrated. The residue (4.5 g) was purified by FC (150 g, DCM:hexane:AcOEt 10:20:0.2) to give the title compound (2.09 g, 5.14 mmol, 75%) ¹H NMR: δ 8.02-7.99 (2H, m), 7.53-7.45 (1H, m), 7.40-7.33 (2H, m), 5.26 (2H, m), 5.11 (1H, dt, J=3.0, 48.0 Hz), 4.46 (1H, dd, J=3.3, 12.5 Hz), 4.21 (1H, m), 3.94 (1H, dd, J=7.7, 12.5 Hz), 3.29 (1H, dd, J=3.3, 7.7 Hz), 2.44-1.44 (4H, m), 0.80 (9H, s), 0.01 (3H, s), 0.00 (3H, s).

### Benzoic acid 2-[5-(tert-butyl-dimethyl-silanyloxy)-3-fluoro-2-methylene-cyclohexylidene]-ethyl ester (12)

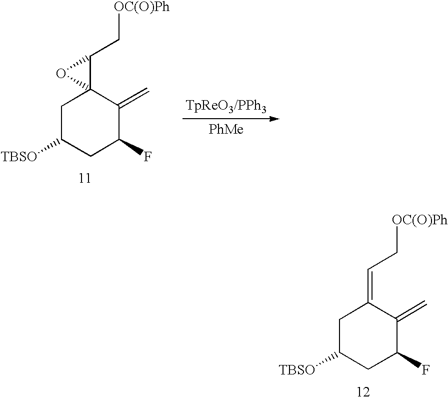

A mixture of tris(3,5-dimethylpyrazoyl)hydridoborate rhenium trioxide (265 mg, 0.50 mmol), triphenylphosphine (158 mg, 0.6 mmol), epoxide 11 (203 mg, 0.5 mmol) and toluene (8 mL) was sealed in an ampule under argon and heated at 100° C. for 14 h. (TLC, 10% AcOEt in hexane, mixture of substrate and product, ca 1:1). Rhenium oxide did not completely solubilized. A solution of triphenylphosphine (158 mg, 0.6 mmol) in toluene (4 mL) was added and the heating continued for 6 h. The reaction mixture was cooled to room temperature filtered through a plug of silica gel and then the residue after evaporation of the solvent was purified by FC (20 g, 5% AcOEt in hexane) to give: 12 (120 mg, 0.31 mmol, 61% of the desire product) and 70 mg of the starting material plus minor contaminations, ca 34%.

### (1Z,3S,5R)-2-[5-(t-butyldimethyl)silanyloxy)-3-fluoro-2-methylene-cyclohexylidene]-ethanol (13)

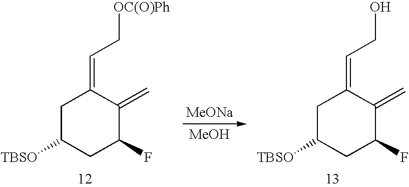

To a solution of a benzoate 12 (150 mg, 0.38 mmol) in methanol (3 mL) was added sodium methoxide (0.5 mL, 15% in methanol). After stirring for 1 h at room temperature water was added (6 mL) and the mixture was extracted with methylene chloride (3×10 mL). The combined organic layers was dried over Na₂SO₄ and evaporated to dryness. The residue (0.2 g) was purified by FC (20 g, 15% AcOEt in hexane) to give 13 (80 mg, 0.28 mmol, 73% of the product).

### (1R,3Z,5S)-t-butyl-[3-(2-chloro-ethylidene)-5-fluoro-4-methylene-cyclohexyloxy]-dimethylsilane (21)

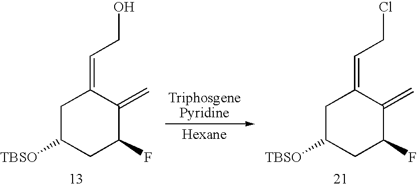

To a solution of 13 (8.07 g, 28.2 mmol) and triphosgene (4.18 g, 14.1 mmol) in hexane (150 mL) at 0° C. was added over 30 min a solution of pyridine (4.5 mL, 55.6 mmol) in hexane (20 mL) and the reaction mixture was stirred at this temperature for 30 min and at room temperature for another 30 min. The reaction mixture was washed with CuSO₄ aq (3×200 mL). The combined aqueous layers were back-extracted with hexane (2×100 mL). The organic layers were combined, dried (MgSO₄), and concentrated in vacuo to give the title compound (9.0 g, overweight). This material was used immediately in the next step without further purification. [α]²⁵_{D} +73.0° (c 0.28, CHCl₃); IR (CHCl₃) 1643, 838 cm⁻¹; ¹H-NMR δ 0.08 (s, 6H), 0.88 (s, 9H), 1.84-2.03 (m, 1H), 2.12 (br s, 1H), 2.24 (m, 1H), 2.48 (br d, J=13 Hz, 1H), 4.06-4.26 (m, 3H), 5.10 (br d, J=48 Hz), 5.16 (s, 1H), 5.35 (s, 1H), 5.63 (br t, J=6 Hz, 1H).

### (1S,3Z,5R)-1-fluoro-5-(t-butyldimethyl)silanyloxy)-2-methenyl-3-(diphenylphosphinoyl)ethylidene cyclohexane (6)

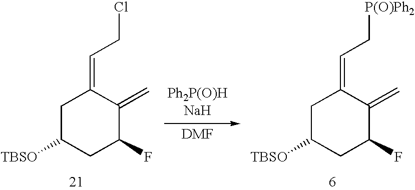

Diphenylphosphine oxide (6.70 g, 33.1 mmol) was added portionwise, over 15 min to a suspension of NaH (1.33 g, 33.1 mmol, 60% dispersion in mineral oil) in DMF (50 mL) at 10° C. The resulting solution was stirred at room temperature for 30 min and cooled to −60° C. The solution of crude 21 (9.0 g) in DMF (20 mL) was then added dropwise. The reaction mixture was stirred at −60° C. for 2 h and at room temperature for 1 h, diluted with diethyl ether (600 mL) and washed with water (3×200 mL). The aqueous layers were extracted with diethyl ether (200 mL). The combined organic layers were dried (MgSO₄) and concentrated under reduced pressure to give white solid. The crude product was recrystallized from diisopropyl ether (25 mL). The resulting solid was collected by filtration, washed with cold diisopropyl ether (5 mL) and dried under high vacuum to give the title compound (7.93 g). The mother liquor was concentrated and the residue was subjected to chromatography on silica gel (50 g, 30%-50% AcOEt in hexane) to give title compound (2.22 g). Thus the total yield of the of 6 was (10.1 g, 21.5 mmol, 76% overall from 13. [α]²⁵_{D} +50.2° (c 0.84, CHCl₃); IR (CHCl₃) 835, 692 cm⁻¹; UVλₘₐₓ (ethanol) 223 (ε 22770), 258 (1950), 265 (1750), 272 nm (1280); MS, m/e 470 (M⁺), 455 (4), 450 (8), 413 (98), 338 (9), 75 (100); ¹H-NMR: δ 0.02 (s, 6H), 0.84 (s, 9H), 1.76-1.93 (m, 1H), 2.16 (m, 2H), 2.42 (br d, 1H), 3.28 (m, 2H), 4.01 (m, 1H), 5.02 (dm, J=44 Hz, 1H), 5.14 (s, 1H), 5.30 (s, 1H), 5.5 (m, 1H), 7.5 (m, 6H), 7.73 (m, 4H). Analysis Calcd for C₂₇H₃₆O₂FPSi: C, 68.91; H, 7.71; F, 4.04. Found: C, 68.69; H, 7.80; F, 3.88.

### 2. Larger Scale Synthesis of the A-Ring Precursor

### (2R,3S,7S)-[7-(t-butyldimethyl)silanyloxy)-4-methylene-1-oxa-spiro[2.5]oct-2-yl]-methanol (8)

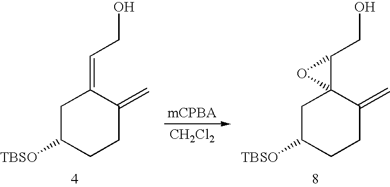

To a stirred solution of crude (S)-(Z)-2-[5-(tert-butyldimethyl)silanyloxy)-2-methylene-cyclohexylidene]-ethanol (4) (13.5 g, ca 40 mmol) in dichloromethane (100 mL) at room temperature, was added AcONa (4.5 g, 54.8 mmol), followed by 77% mCPBA (8.96 g, 40.0 mmol) at +5° C. The reaction mixture was then stirred at +5° C. for 1.5 h, diluted with hexane (500 mL), washed with water (200 mL) and NaHCO₃ (2×200 mL) and dried over Na₂SO₄. The residue after evaporation of solvent (12.36 g) was used for the next step without further purification. ¹H-NMR: δ 0.063 and 0.068 (2s, 6H), 0.88 (s, 9H), 1.38-1.49 (m, 1H), 1.54 (m, 1H, OH), 1.62 (m, 1H), 1.96 (m, 3H), 2.43 (m, 1H), 3.095 (t, 1H, J=5.6 Hz), 3.60 (m, 2H), 3.86 (m, 1H), 4.91 (m, 1H).

### Benzoic acid (2R,3S,7S)-7-(t-butyldimethyl)silanyloxy)-4-methylene-1-oxa-spiro[2.5]oct-2-yl methyl ester (9)

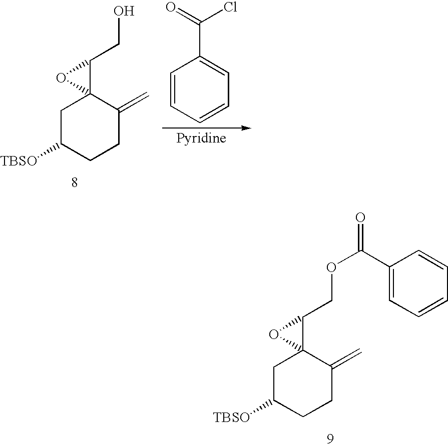

To a stirred solution of (2R,3S,7S)-[7-(tert-butyldimethyl)silanyloxy)-4-methylene-1-oxa-spiro[2.5]oct-2-yl]-methanol (8) (12.36 g) in pyridine (50 mL) at room temperature, was added benzoyl chloride (8.5 mL, 73 mmol) and the reaction mixture was stirred for 2 h. Water (60 mL) was added and after stirring for 45 min at room temperature the mixture was diluted with hexane (250 mL), washed with NaHCO₃aq (2×250 mL), brine (250 mL) and dried over Na₂SO₄. The residue after evaporation of the solvent (15.28 g) was used for the next step without further purification. ¹H NMR: δ 8.04-7.80 (2H, m), 7.56-7.50 (1H, m), 7.44-7.37 (2H, m), 4.94 (1H, brs), 4.92 (1H, brs), 4.32 (1H, dd, J=4.8, 11.9 Hz), 4.14 (1H, dd, J=6.2, 11.9 Hz), 3.83 (1H, m), 3.21 (1H, dd, J=4.8, 6.2 Hz), 2.42 (1H, m), 2.04-1.90 (3H, m), 1.64-1.34 (2H, m), 0.83 (9H, s), 0.02 (3H, s), 0.01 (3H, s).

### Benzoic acid (2R,3S,5R,7S)-7-(t-butyldimethyl)silanyloxy)-5-hydroxy-4-methylene-1-oxa-spiro[2.5]oct-2-yl methyl ester (10)

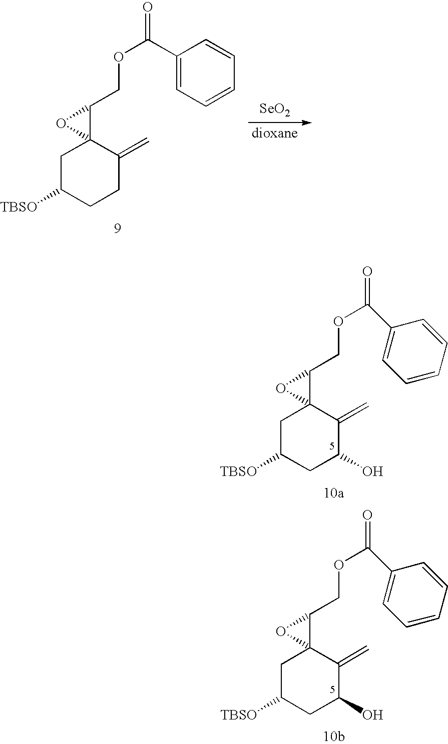

To a stirred solution of benzoic acid (2R,3S,7S)-7-(tert-butyldimethyl)silanyloxy)-4-methylene-1-oxa-spiro[2.5]oct-2-yl methyl ester (9) (15.28 g)) in dioxane (450 mL) at 85° C. was added selenium dioxide (4.26 g, 38.4 mmol), followed by tert-butyl hydrogen peroxide (7.7 mL, 38.4 mmol, 5-6 M in nonane) and the reaction mixture was stirred at 85° C. for 13 h, after which selenium dioxide (2.39 g, 21.5 mmol) was added, followed by tert-butyl hydrogen peroxide (4.3 mL, 21.5 mmol, 5-6 M in nonane) and the reaction mixture was stirred at 85° C. for additional 24 h. The mixture was filtered off through a plug of silica gel (0.5 kg, AcOEt). The solvent was removed under vacuum and the residue was dissolved in AcOEt (250 mL) and washed with water (3×100 mL). The organic layer was dried over Na₂SO₄ and evaporated under vacuum. The residue (16 g) was purified by flash chromatography (0.5 kg, 10, 15 and 20% AcOEt in hexane) to give: Fraction A (1.1 g, of a starting material); Fraction B (0.78 g, of 10b); Fraction C (3.01 g, 65:35 (10b:10a); Fraction D (6.22 g, 5:95 (10b:10a); Fraction D was crystallized two times (each time using the remaining oil) from hexane to give pale yellow solid Fraction E (6.0 g in total) and yellow-red oil Fraction F (0.2 g in total). Fractions C and F were purified by flash chromatography (300 g, 20% AcOEt in hexane) to give: Fraction G (0.8 g, of 10b); Fraction H (2.4 g, 8:92 10b:10a). Fraction H was crystallized two times (each time using the remaining oil) from hexane to give pale yellow solid Fraction I (2.2 g in total) and yellow-red oil Fraction J (0.2 g in total). Fractions E and I were combined to give 10a (8.2 g, 20.3 mmol, 50.7% total yield from compound 4). [α]²²_{D} −10.6° (c 0.35, EtOH); ¹H NMR: δ 8.04 (2H, m), 7.58 (1H, m), 7.46 (2H, m), 5.32 (1H, br s), 5.18 (1H, br s), 4.33 (1H, dd, J=5.2, 11.9 Hz), 4.21 (1H, dd, J=6.0, 11.9 Hz), 4.14 (1H, ddd, J=2.6, 4.9, 10.0 Hz), 3.94 (1H, m), 3.25 (1H, dd, J=5.5, 5.9 Hz), 2.38 (1H, m), 2.05 (1H, t, J=11.5 Hz), 1.64 (1H, ddd, J=1.9, 4.3, 12.2 Hz), 1.52 dt, J=11.1, 11.7 Hz), 1.28 (1H, m), 0.87 (9H, s), 0.07 (3H, s), 0.06 (3H, s); ¹³C NMR: 166.31 (0), 145.52 (0), 133.29 (1), 129.65 (1), 129.54 (0), 128.46 (1), 107.44 (2), 68.51 (1), 65.95 (1), 62.75 (2), 61.62 (1), 61.09 (0), 45.23 (2), 44.33 (2), 25.72 (3), 18.06 (0), 4.72 (3); MS HR-ES: Calcd. For C₂₂H₃₂O₅Si: M+Na 427.1911 Found: 427.1909.

### Benzoic acid (2R,3S,5S,7R)-7-(t-butyldimethyl)silanyloxy)-5-fluoro-4-methylene-1-oxa-spiro[2.5]oct-2-ylmethyl ester (11)

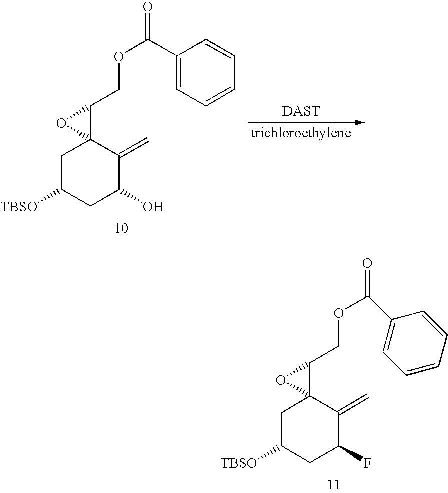

To a stirred solution of diethylaminosulfur trifluoride (16.5 mL, 126.0 mmol) in trichloroethylene (140 mL) was added a solution of benzoic acid (2R,3S,5R,7S)-7-(tert-butyldimethyl)silanyloxy)-5-hydroxy-4-methylene-1-oxa-spiro[2.5]oct-2-yl methyl ester (10a) (18.7 g, 46.2 mmol) in trichloroethylene (100 mL at −75° C. After stirring for 20 min. at −75° C. methanol (40 mL) was added, followed by NaHCO_{3aq} (50 mL) and the resulting mixture was diluted with hexane (700 mL) and washed with NaHCO_{3aq} (600 mL), dried over Na₂SO₄ and concentrated on rotary evaporator. The residue (25.6 g) was purified by flash chromatography (500 g, DCM:hexane:AcOEt 10:20:0.2) to give 11 (13.9 g, 34.2 mmol, 74%); [α]²⁹_{D} +38.9° (c 0.8, CHCl₃); ¹H NMR: δ 8.07 (2H, m), 7.57 (1H, m), 7.44 (2H, m), 5.33 (2H, m), 5.20 (1H, dt, J=2.9, 48 Hz), 4.55 (1H, dd, J=3.2, 12.3 Hz), 4.29 (1H, m), 4.02 (1H, dd, J=7.9, 12.3 Hz), 3.37 (1H, dd, J=3.2, 7.7 Hz), 2.45 (1H, m), 2.05 (1H, t, J=11.9 Hz), 1.73 (1H, dm), 1.62 (1H, m), 0.88 (9H, s), 0.08 (3H, s), 0.06 (3H, s); ¹³C NMR: 166.25 (0), 139.95 (0, d, J=17 Hz), 132.97 (1), 129.75 (0), 129.62 (1), 128.24 (1), 116.32 (2, d, J=9 Hz), 92.11 (1, d, J=162 Hz), 65.23 (1), 63.78 (2), 62.29 (1), 60.35 (0), 44.38 (2), 41.26 (2, d, J=23 Hz), 25.81 (3), 18.13 (0), −4.66 (3); MS HR-ES: Calcd. For C₂₂H₃₁O₄SiF: M+H407.2049 Found: 407.2046.

### (1E,3S,5R)-2-[5-(tert-Butyldimethyl)silanyloxy)-3-fluoro-2-methylene-cyclohexylidene]-ethanol (13a)

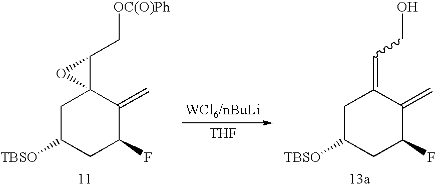

Tungsten hexachloride (36.4 g, 91 mmol) was added at −75° C. to THF (800 mL). The temperature was adjusted to −65° C. and nBuLi (73 mL, 182.5 mmol, 2.5M solution in hexane) was added maintaining temperature below −20° C. After the addition was completed the reaction mixture was allowed to come to room temperature and it was stirred for 30 min., cooled down to 0° C., when a solution of benzoic acid (2R,3S,5S,7R)-7-(tert-butyldimethyl)silanyloxy)-5-fluoro-4-methylene-1-oxa-spiro[2.5]oct-2-yl methyl ester (11) (18.5 g, 45.5 mmol) in THF (50 mL) was added. Thus formed mixture was allowed to come to room temperature (2 h) and stirred for 16 h. Methanol (400 mL) was added followed by sodium methoxide (250 mL, 15% in methanol), the resulting mixture was stirred for 30 min then diluted with AcOEt (1 L) and washed with water (1 L) and brine (500 mL). The residue (21.6 g) after evaporation of the dried (Na₂SO₄) solvent was used for the next step without further purification.

¹H-NMR (CDCl₃); δ 0.09 (s, 6H), 0.81 (s, 9H), 1.80-2.22 (m, 3H), 2.44 (m, 1H), 4.10 (m, 1H), 4.14 (d, 2H, J=6.9 Hz), 4.98 (br s, 1H), 5.10 (d, 1H, J=50.0 Hz), 5.11 (s, 1H), 5.79 (t, 1H, J=6.8 Hz).

### (1Z,3S,5R)-2-[5-(tert-Butyldimethyl)silanyloxy)-3-fluoro-2-methylene-cyclohexylidene]-ethanol (13)

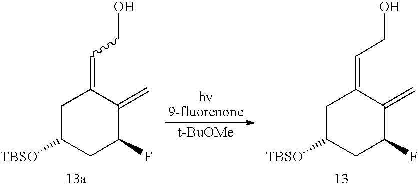

A solution of (1E,3S,5R)-2-[5-(tert-butyldimethyl)silanyloxy)-3-fluoro-2-methylene-cyclohexylidene]-ethanol (13a) (21.6 g, crude containing ca 10% of the Z isomer) and 9-fluorenone (1.8 g, 10 mmol) in tert-Butyl-methyl ether (650 mL) was irradiated with 450 W hanovia lamp with uranium core filter for 8 h. The residue after evaporation of solvent (23.95 g) was purified by flash chromatography (750 g, 5%, 20%, AcOEt in hexane) to give the title compound 13 (10.4 g, 36.3 mmol, 80% from 11). [α]³⁰_{D} +40.1° (c 0.89, EtOH)

¹H-NMR: δ 5.65 (1H, t, J=6.8 Hz), 5.31 (1H, dd, J=1.5, 1.7 Hz), 5.10 (1H, ddd, J=3.2, 6.0, 49.9 Hz), 4.95 (1H, d, J=1.7 Hz), 4.28 (1H, dd, J=7.3, 12.6 Hz), 4.19 (1H, ddd, J=1.7, 6.4, 12.7 Hz), 4.15 (1H, m), 2.48 (1H, dd, J=3.8, 13.0 Hz), 2.27-2.13 (2H, m), 1.88 (1H, m), 0.87 (9H, s), 0.07 (6H, s). ¹³C-NMR: 142.54 (0, d, J=17 Hz), 137.12 (0, d, J=2.3 Hz), 128.54 (1), 115.30 (2, d, J=10 Hz), 92.11 (1, d, J=168 Hz), 66.82 (1, d, J=4.5 Hz), 59.45 (2), 45.15 (2), 41.44 (2, d, J=21 Hz), 25.76 (3), 18.06 (0), −4.75(3), −4.85(3).

### (1R,3Z,5S)-t-butyl-[3-(2-chloro-ethylidene)-5-fluoro-4-methylene-cyclohexyloxy]-dimethylsilane (21)

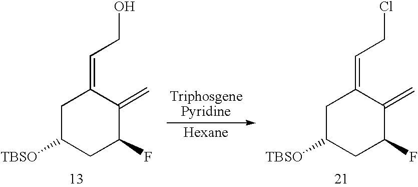

To a solution of 13 (8.07 g, 28.2 mmol) and triphosgene (4.18 g, 14.1 mmol) in hexane (150 mL) at 0° C. was added over 30 min a solution of pyridine (4.5 mL, 55.6 mmol) in hexane (20 mL) and the reaction mixture was stirred at this temperature for 30 min and at room temperature for another 30 min. The reaction mixture was washed with CuSO₄ aq (3×200 mL). The combined aqueous layers were back-extracted with hexane (2×100 mL). The organic layers were combined, dried (MgSO₄), and concentrated in vacuo to give the title compound (9.0 g, overweight). This material was used immediately in the next step without further purification. [α]²⁵_{D} +73.0° (c 0.28, CHCl₃); IR (CHCl₃) 1643, 838 cm⁻¹; ¹H-NMR δ 0.08 (s, 6H), 0.88 (s, 9H), 1.84-2.03 (m, 1H), 2.12 (br s, 1H), 2.24 (m, 1H), 2.48 (br d, J=13 Hz, 1H), 4.06-4.26 (m, 3H), 5.10 (br d, J=48 Hz), 5.16 (s, 1H), 5.35 (s, 1H), 5.63 (br t, J=6 Hz, 1H).

### (1S,3Z,5R)-1-fluoro-5-(t-butyldimethyl)silanyloxy)-2-methenyl-3-(diphenylphosphinoyl)ethylidene cyclohexane (6)

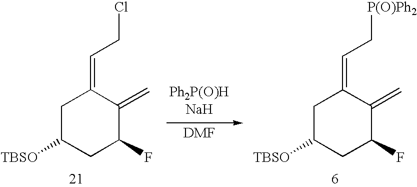

Diphenylphosphine oxide (6.70 g, 33.1 mmol) was added portionwise, over 15 min to a suspension of NaH (1.33 g, 33.1 mmol, 60% dispersion in mineral oil) in DMF (50 mL) at 10° C. The resulting solution was stirred at room temperature for 30 min and cooled to −60° C. The solution of crude 21 (9.0 g) in DMF (20 mL) was then added dropwise. The reaction mixture was stirred at −60° C. for 2 h and at room temperature for 1 h, diluted with diethyl ether (600 mL) and washed with water (3×200 mL). The aqueous layers were extracted with diethyl ether (200 mL). The combined organic layers were dried (MgSO₄) and concentrated under reduced pressure to give white solid. The crude product was recrystallized from diisopropyl ether (25 mL). The resulting solid was collected by filtration, washed with cold diisopropyl ether (5 mL) and dried under high vacuum to give the title compound (7.93 g). The mother liquor was concentrated and the residue was subjected to chromatography on silica gel (50 g, 30%-50% AcOEt in hexane) to give title compound (2.22 g). Thus the total yield of the of 6 was (10.1 g, 21.5 mmol, 76% overall from 13. [α]²⁵_{D} +50.2° (c 0.84, CHCl₃); IR (CHCl₃) 835, 692 cm⁻¹; UVλₘₐₓ (ethanol) 223 (ε 22770), 258 (1950), 265 (1750), 272 mm (1280); MS, m/e 470 (M⁺), 455 (4), 450 (8), 413 (98), 338 (9), 75 (100); ¹H-NMR: δ 0.02 (s, 6H), 0.84 (s, 9H), 1.76-1.93 (m, 1H), 2.16 (m, 2H), 2.42 (br d, 1H), 3.28 (m, 2H), 4.01 (m, 1H), 5.02 (dm, J=44 Hz, 1H), 5.14 (s, 1H), 5.30 (s, 1H), 5.5 (m, 1H), 7.5 (m, 6H), 7.73 (m, 4H). Analysis Calcd for C₂₇H₃₆O₂FPSi: C, 68.91; H, 7.71; F, 4.04. Found: C, 68.69; H, 7.80; F, 3.88.

### Example 3

### 1. Synthesis of C,D-Ring/Side Chain Precursor

### (S)-2-((1R,3aR,4S,7aR)-4-hydroxy-7a-methyl-octahydro-inden-1-yl)-propionaldehyde (14)

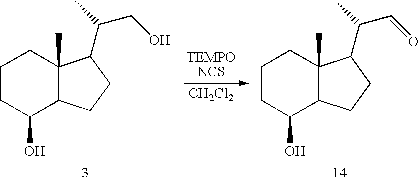

A 250-mL flask was charged with 0.99 g (4.67 mmol) of Lythgoe diol (3), 75 mg (0.48 mmol) of TEMPO, 146 mg (0.53 mmol) of tetrabutylammonium chloride hydrate, and dichloromethane (50 mL). To this vigorously stirred solution was added a buffer solution (50 mL) prepared by dissolving sodium hydrogen carbonate (4.2 g) and potassium carbonate (0.69 g) in a volume of 100 mL of water. The mixture was stirred vigorously and 839 mg (6.28 mmol) of N-chlorosuccinimide was added. TLC (1:2, ethyl acetate-heptane) showed the gradual conversion of educt (R_{f} 0.32) to the aldehyde 14 (R_{f} 0.61). After 18 h an additional quantity of 830 mg (6.28 mmol) of N-chlorosuccinimide was added and one hour later 20 mg of TEMPO was added and the mixture was stirred for 24 h. The organic layer was separated and the aqueous layer re-extracted with dichloromethane (3×50 mL). The combined organic extracts were washed with brine, dried and concentrated in vacuo. The residue was purified by column chromatography (SiO₂, ethyl acetate/heptane=1:3) to furnish 876 mg of crude aldehyde 14 (89%) ¹H NMR: δ 9.58 (1H, d, J=2.8 Hz), 4.12 (1H, m), 2.50-2.30 (1H, m), 2.10-1.10 (13H, m), 1.11 (3H, d, J=7.0 Hz), 0.99 (3H, s).

### (1R,3aR,4S,7aR)-7a-methyl-1-((S)-1-methyl-2-oxo-ethyl)-octahydroinden-4-yl ester (15)

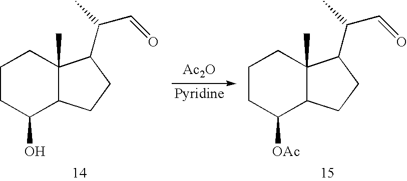

The crude 14 (255 mg, 1.21 mmol) was dissolved in pyridine (1 mL), the soln. cooled in an ice bath and DMAP (5 mg) and acetic anhydride (0.5 mL) were added. The mixture was stirred at room temperature for 24 h then diluted with water (10 mL), stirred for 10 min and equilibrated with ethyl acetate (30 mL). The organic layer was washed with a mixture of water (10 mL) and 1 N sulfuric acid (14 mL), then with water (10 mL) and saturated sodium hydrogen carbonate solution (10 mL), then dried and evaporated. The resulting residue (201 mg) was chromatographed on a silica gel column using 1:4 ethyl acetate-hexane as mobile phase. The fractions containing the product were pooled and evaporated to give the title compound as a colorless syrup (169 mg, 0.67 mmol, 67%). ¹H NMR (300 MHz, CDCl₃): δ 9.56 (1H, d, J=2.0 Hz), 5.20 (1H, br s), 2.44-2.16 (1H, m), 2.03 (3H, s), 2.00-1.15 (12H, m), 1.11 (3H, d, J=7.0 Hz), 0.92 (3H, s).

### Acetic acid (3aR,4S,7aR)-1-E-ethylidene-7a-methyl-octahydroinden-4-yl ester (16)

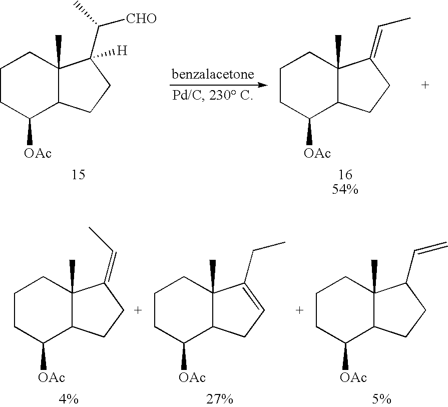

To a solution of aldehyde 15 (480 mg, 1.90 mmol) in diethylether (5 mL) was added 10% Pd on Carbon (25 mg). The suspension was stirred at room temperature for 20 min., filtered through a path of Celite and the filtrate was concentrated in vacuo. To the residue was added benzalacetone (350 mg, 2.40 mmol, distilled) and 10% Pd on Carbon (50 mg). The suspension was degassed by evacuating the flask and refilling with nitrogen (2×). Then the flask was immersed in a 230° C. heating bath for 40 min. After cooling at room temperature the suspension was diluted with ethyl acetate, filtered through a path of Celite and the filtrate was concentrated in vacuo. The residue was purified by column chromatography (SiO₂, ethyl acetate/heptane=1:9) affording 290 mg (68%) of a mixture of CD olefins. GC analysis: 16 (54%); Z isomer (4%); internal olefin (27%); terminal olefin (5%); other impurities (10%).

### (2R,3aR,4S,7aR)-1-E-ethylidene-2-hydroxy-7a-methyl-octahydroinden-4-yl ester (17a) and acetic acid (2S,3aR,4S,7aR)-1-E)-ethylidene-2-hydroxy-7a-methyl-octahydroinden-4-yl ester (17b)

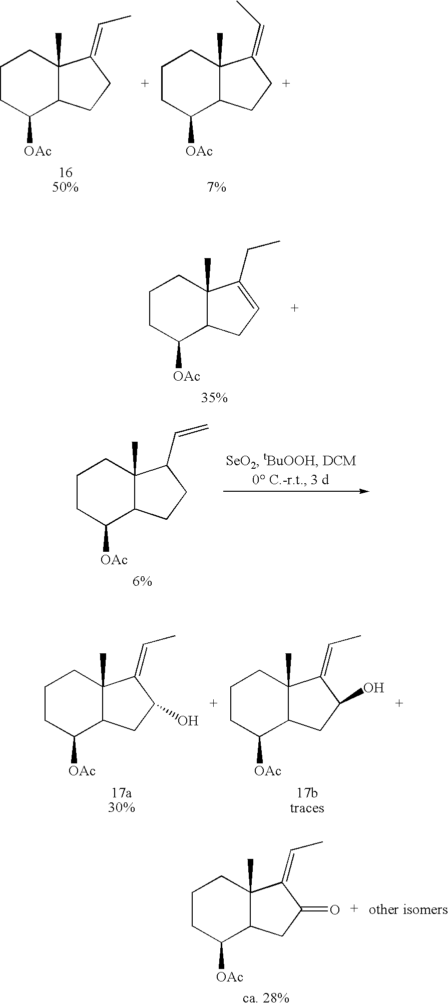

To a suspension of SeO₂ (460 mg, 4.15 mmol) in dichloromethane (30 mL) was added tert.-butylhydroperoxide (9.0 mL, 70 w/w-% solution in water, 65.7 mmol). The suspension was stirred at room temperature for 30 min., cooled at 0° C. and a solution of CD-isomers (9.13 g, 41.1 mmol, contains ca 50% of 16) in dichloromethane (35 mL) was added dropwise within 30 min. The reaction mixture was allowed to reach room temperature overnight and stirring was continued at 30° C. for 2 days. Conversion was checked by GC. The reaction was quenched by addition of water and the aqueous layer was extracted with dichloromethane (3×). The combined organic layers were washed with water (4×), washed with brine, dried (Na₂SO₄), filtered and the filtrate was concentrated in vacuo. The residue was purified by column chromatography (SiO₂, ethyl acetate/heptane=1:3) affording three main fractions: Fraction 1: Ketone (2.08 g, 42% yield); contaminated with 2 impurities; purity 75%; Fraction 2: mixed fraction of alcohol 17a+unwanted isomer (1.32 g); Fraction 3: Alcohol 17a (2.10 g, 42% yield); contaminated with ca. 12% byproduct, but pure enough for further synthesis. Fraction 2 was purified again by column chromatography affording 1.01 g (20% yield) of alcohol 17a contaminated with ca. 20% of an unwanted isomer, but pure enough for further synthesis. *Note: During the oxidation reaction the formation of both isomers 17a and 17b was observed by tlc and GC. After prolonged reaction times the intensity of the lower spot on tlc (mixture of 17b and other isomers) decreased and the formation of ketone was observed. It is important that not only conversion of 16 to alcohol 17a and 17b is complete but also that epimer 17b is completely oxidized to ketone. Epimer 17b can not be separated from unwanted isomers. Retention times on GC: 16 ret. Time=8.06 min; 17 ret. Time=9.10 min; 17b ret. Time=9.30 or 9.34 min; ketone ret. Time=9.60 min. Compound 17a: ¹H NMR: δ 0.94 (s, 3H), 1.30 (m, 1H), 1.40-1.46 (m, 1H), 1.46-1.80 (m, 4H), 1.77 (dd, J=7.2, 1.2 Hz, 3H), 1.80-1.94 (m, 4H), 2.02 (s, 3H), 4.80 (br. s, 1H), 5.23 (m, 1H), 5.47 (qd, J=7.2, 1.2 Hz, 1H). GC-MS: m/e 223 (M−15), 178 (M−60), 163 (M−75). Compound 17b: ¹H NMR: δ 1.24 (s, 3H), 1.38-1.60 (m, 5H), 1.68-1.88 (m, 3H), 1.72 (dd, J=7.2, 1.2 Hz, 3H), 1.99 (ddd, J=11.0, 7.0, 3.7 Hz, 1H), 2.03 (s, 3H), 2.26 (m, 1H), 4.36 (m, 1H), 5.14 (m, 1H), 5.30 (qd, J=7.2, 1.2 Hz, 1H). GC-MS: m/e 223 (M−15), 178 (M−60), 163 (M−75).

### Reduction of Ketone to Alcohol 17b

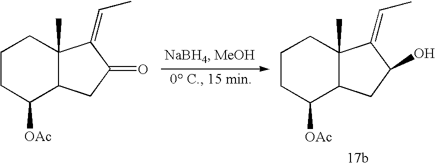

A solution of ketone (2.08 g, contaminated with 2 impurities) in methanol (8 mL) was cooled at 0° C. and sodium borohydride (0.57 g, 15.1 mmol) was added in portions. After stirring at 0° C. for 1 h, tlc showed complete conversion (no UV active compound visible on tlc). The reaction mixture was quenched by addition of sat. aqueous NH₄Cl solution (30 mL). Water was added and the aqueous layer was extracted with ethyl acetate (3×). The combined organic layers were washed with brine, dried (Na₂SO₄), filtered and the filtrate was concentrated in vacuo. The residue was purified by column chromatography (SiO₂, ethyl acetate/heptane=1:3) affording alcohol 17b (1.20 g, 24% over two steps) as a colorless oil.

### Acetic acid (3aR,4S,7aR)-7a-methyl-1-(1-(R)-methyl-3-oxo-propyl)-3a,4,5,6,7,7a-hexahydro-3H-inden-4-yl ester (18)

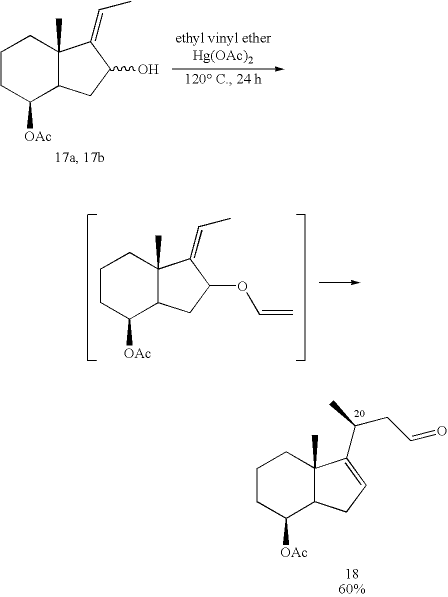

Both alcohols 17a and 17b (4.3 g, 18.1 mmol, purity 90%) were converted to compound 18 in three batches. To a solution of 17a (2.1 g, 8.82 mmol) in ethyl vinyl ether (20 mL) was added Hg(OAc)₂ (2.23 g, 7.00 mmol). The suspension was poured into a pyrex pressure tube, flushed with N₂ and closed tightly. The mixture was stirred at 120° C. for 24 h, cooled at room temperature and filtered. The filtrate was concentrated in vacuo and the residue was combined with the crude product of the two other batches and purified twice* by column chromatography (SiO₂, ethyl acetate/heptane=1:4) affording aldehyde 18 (2.58 g, 60%) as a slightly yellow oil. The product solidified upon storage in the freezer. A second purification by column chromatography was advantageous due to the byproducts present in the starting material.

To a solution of epimers 17a and 17b (173 mg, 0.73 mmol) in toluene (2 mL) was added a catalytic amount of [Ir(COD)Cl]₂ (5 mg), Na₂CO₃ (46 mg, 0.44 mmol) and vinyl acetate (0.13 mL, 1.45 mmol). After heating the suspension at 100° C. for 2 h, tlc indicates ca. 20% conversion to intermediate. (J. Am. Chem. Soc., 2002, 134, 1590-1591.) More vinyl acetate (0.15 mL) was added and stirring at 100° C. was continued for 18 h. According tlc a mixture of intermediate and 18 was formed but conversion of the starting material was still not complete. More vinyl acetate (2 mL) was added and stirring at 100° C. was continued for 24 h. Tlc shows complete conversion of the starting material to a mixture of intermediate and aldehyde 18. The suspension was concentrated in vacuo and the residue was purified by column chromatography (SiO₂, ethyl acetate/heptane=1:9) affording 60 mg of intermediate (31%) and 45 mg of aldehyde 18 (23%). ¹H NMR: δ 1.02 (s, 3H), 1.14 (d, J=7.1 Hz, 3H), 1.36 (M, 1H), 1.47-1.62 (m, 2H), 1.72-1.90 (m, 4H), 2.03 (s, 3H), 2.02-2.14 (m, 2H), 2.33 (ddd, J=16.2, 7.3, 2.6 Hz, 1H), 2.53 (ddd, J=16.2, 5.8, 1.8 Hz, 1H), 2.72 (m, 1H), 5.19 (m, 1H), 5.40 (m, 1H), 9.68 (s, 1H).

### 5(R)-((3aR,4S,7aR)-4-acetoxy-7a-methyl-3a,4,5,6,7,7a-hexahydro-3H-inden-1-yl)-hex-2-E-enoic acid ethyl ester (19)

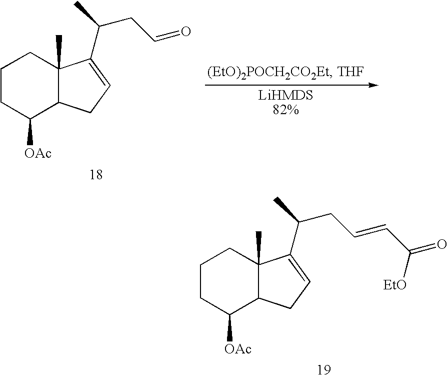

Aldehyde 18 (2.24 g, 8.47 mmol) and triethyl phosphonoacetate (5.74 g, 25.6 mmol, 3 eq.) were dissolved under N₂ atmosphere in THF (40 mL, freshly distilled over Na/benzophenone). The mixture was cooled at −100° C. and a solution of LiHMDS in hexanes (16.8 mL, 1 M solution, 2 eq.) was added dropwise within 20 min. After stirring at −100° C. [symbol] −78° C. for 70 min. the reaction was quenched by dropwise addition of water (10 mL) and subsequently addition of sat. NH₄Cl solution (10 mL). Water was added and it was extracted with tert. butyl methyl ether (3×). The combined organic layers were washed with water (2×), brine (1×), dried (Na₂SO₄), filtered and the filtrate was concentrated in vacuo. The residue was purified by column chromatography (SiO₂, ethyl acetate/heptane=1:10) affording ester E-19 (2.15 g, 76%) as a colorless oil; purity according NMR: >95% (no Zisomer detected). ¹H NMR: δ 0.99 (s, 3H), 1.06 (d, J=7.2 Hz, 3H), 1.27 (t, J=7.1 Hz, 3H), 1.36 (td, J=13.3, 4.0 Hz, 1H), 1.46-1.62 (m, 2H), 1.72-1.90 (m, 4H), 1.96-2.17 (m, 3H), 2.03 (s, 3H), 2.22-2.39 (m, 2H), 4.17 (q, J=7.2 Hz, 2H), 5.20 (br. s, 1H), 5.37 (br. s, 1H), 5.78 (dm, J=15.4 Hz, 1H), 6.88 (dt, J=15.4, 7.3 Hz, 1H). HPLC: purity >99% (218 nm). HPLC-MS: m/e 357 (M+23), 275 (M−59).

### (3aR,4S,7aR)-1-((S,E)-5-ethyl-5-hydroxy-1-methyl-hept-3-enyl)-7a-methyl-3a,4,5,6,7,7a-hexahydro-3H-inden-4-ol (20)

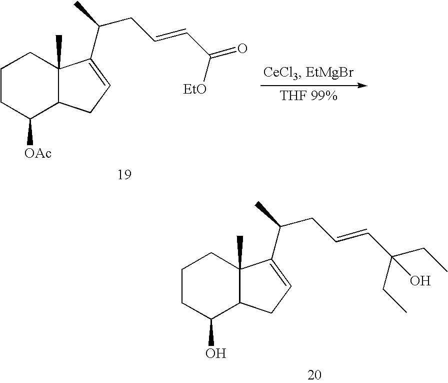

CeCl₃×7H₂O (29.1 g) was dried in vacuo (10⁻³ mbar) in a three-necked flask at 160° C. for 6 h affording anhydrous CeCl₃ (18.7 g, 76.0 mmol, 12 eq.). After cooling at room temperature the flask was purged with nitrogen. THF (200 mL, freshly distilled over Na/benzophenone) was added and the mixture was stirred at room temperature for 18 h. Subsequently the suspension was cooled at 0° C. and a solution of EtMgBr in THF (75 mL, 1 M solution) was added dropwise within 20 min. After stirring the light brown suspension at 0° C. for 2 h a solution of ester E-19 (2.15 g, 6.42 mmol) in THF (30 mL, freshly distilled over Nalbenzophenone) was added dropwise within 10 min. After stirring at 0° C. for 30 min. tlc showed complete conversion and the reaction was quenched by addition of water (60 mL). More water was added and the mixture was extracted with 50% ethyl acetate in heptane (3×). The combined organic layers were washed with sat. NaHCO₃ solution (2×), brine (1×), dried (Na₂SO₄), filtered and the filtrate was concentrated in vacuo affording a slightly yellow oil. The crude product (2.4 g) was combined with a 2^{nd} batch (600 mg crude 20 obtained from 550 mg 19). Purification by column chromatography (SiO₂, ethyl acetate/heptane=1:3) afforded 20 (2.45 g, 99%) as a colorless oil. ¹H NMR: δ 0.84 (t, J=7.3 Hz, 6H), 1.04 (d, J=7.2 Hz, 3H), 1.05 (s, 3H), 1.23-1.60 (m, 9H), 1.67-2.02 (m, 6H), 2.12-2.32 (m, 3H), 4.17 (m, 1H), 5.33 (m, 1H), 5.35 (dm, J=15.4 Hz, 1H), 5.51 (ddd, J=15.4, 7.4, 6.5 Hz, 1H). HPLC: purity=98% (212 nm). HPLC-MS: m/e 330 (M+24), 289 (M−17), 271 (M−35).

### (3aR,4S,7aR)-1-((S,E)-5-ethyl-5-hydroxy-1-methyl-hept-3-enyl)-7a-methyl-3a,4,5,6,7,7a-hexahydro-3H-inden-4-one (5)

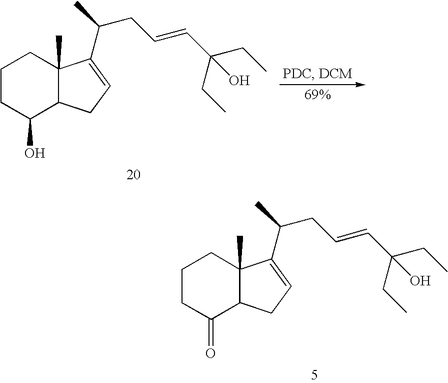

A solution of diol 20 (465 mg, 1.52 mmol) in dichloromethane (30 mL) was cooled in an ice-bath and treated portion-wise with pyridinium dichromate (1.28 g, 3.40 mmol, 2.2 eq.). The reaction mixture was stirred at 0° C. for 6 h and at room temperature for 18 h. The reaction mixture was filtered through a path of Celite. The filtercake was washed with dichloromethane and the combined filtrates were concentrated in vacuo. The residue was purified by column chromatography (SiO₂, 25% ethyl acetate in heptane) affording ketone 5 (320 mg, 69%) as a colorless oil. ¹H NMR: δ 0.82 (s, 3H), 0.85 (br. t, J=7.2 Hz, 6H), 1.05 (d, J=6.9 Hz, 3H), 1.34 (br. s, 1H), 1.52 (br. q, J=6.9 Hz, 4H), 1.65 (td, J=12.1, 5.6 Hz, 1H), 1.84-1.93 (m, 1H), 1.93-2.16 (m, 4H), 2.16-2.33 (m, 4H), 2.42 (ddt, J=15.4, 10.4, 1.6 Hz, 1H), 2.82 (dd, J=10.4, 6.0 Hz, 1H), 5.30 (m, 1H), 5.38 (dm, J=15.6 Hz, 1H), 5.54 (ddd, J=15.6, 7.1, 6.0 Hz, 1H).

### 2. Larger Scale Synthesis of C,D-Ring/Side Chain Precursor

### Acetic acid (1R,3aR,4S,7aR)-1-((S)-1-hydroxypropan-2-yl)-7a-methyl-octahydro-1H-inden-4-yl ester (3a)

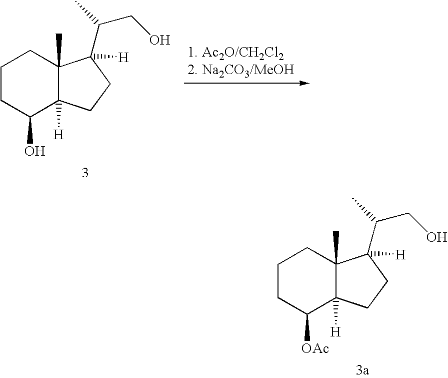

A 1 l round bottom flask equipped with stirring bar and Claisen adapter with rubber septum was charged with Lythgoee diol 3 (38.41 g, 180.9 mmol), dichloromethane (400 mL), pyridine (130 mL) and DMAP (5.00 g, 40.9 mmol). Acetic anhydride (150 mL) was added slowly and the mixture was stirred at room temperature for 14.5 h. Methanol (70 mL) was added dropwise (exothermic reaction) to the reaction mixture and the solution was stirred for 30 min. Water (1 L) was added and the aqueous layer was extracted with dichloromethane (2×250 mL). The extracts were washed with 1N HCl (200 mL) and solution of NaHCO₃ (200 mL), dried (Na₂SO₄) and evaporated to dryness with toluene (150 mL). The residue was dissolved in methanol (300 mL) and sodium carbonate (40.0 g) was added. The suspension was stirred for 24 h. Additional portion of sodium carbonate (10.0 g) was added and the reaction mixture was stirred for 18 h. Methanol was removed on a rotary evaporator. Water (500 mL) was added and the mixture was extracted with ethyl acetate (3×250 mL), dried Na₂SO₄) and concentrated in vacuo. The residue was purified by FC (0.4 kg of silica gel, 20%, 30% hexane-ethyl acetate) to give the title compound 3a (45 g, 98%). ¹H NMR (DMSO-D6) 5.03 (1H, br s), 4.26 (1H, dd, J=5.9, 5.1 Hz), 3.42-3.36 (1H, m), 3.10-3.02 (1H, m), 1.99 (3H, s), 1.96-1.91 (1H, m), 1.77-1.58 (3H, m), 1.50-1.08 (9H, m), 0.93 (3H, d, J=6.6 Hz), 0.85 (3H, s).

### Acetic acid (1R,3aR,4S,7aR)-7a-methyl-1-((S)-oxopropan-2-yl)-octahydro-1H-inden-4-yl ester (15)

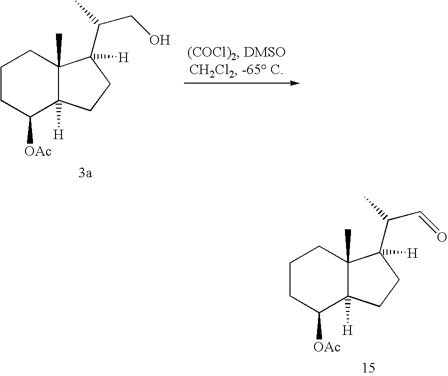

To a cooled solution (−65° C.) of oxalyl chloride (17 mL, 195 mmol) in dichloromethane (150 mL) was added within 35 min. a solution of DMSO (27 mL, 380 mmol) in dichloromethane (200 mL), keeping the temperature below −65° C. After complete addition stirring at −65° C. was continued for 15 min. Subsequently a solution of acetic acid (1R,3aR,4S,7aR)-1-((S)-1-hydroxypropan-2-yl)-7a-methyl-octahydro-1H-inden-4-yl ester 3a (41 g, 161 mmol) in dichloromethane (300 mL) was added dropwise within 80 min., keeping the temperature below −65° C. During addition a solid precipitated. After complete addition stirring at −65° C. was continued for 1 h. Subsequently a solution of triethylamine (110 mL) in dichloromethane (200 mL) was added dropwise within 30 min. After complete addition stirring at −65° C. was continued for 45 min. The cooling bath was removed and the reaction mixture was allowed to warm to 5° C. within 1 h. Dichloromethane (ca. 600 mL) was removed by distillation under reduced pressure and to the residue was added water (600 mL) and tert-Butyl-methyl ether (500 mL). The organic layer was separated and the aqueous layer was extracted with tert-Butyl-methyl ether (2×200 mL). The combined organic layers were dried (Na₂SO₄), filtered and concentrated in vacuo. The residue was purified by column chromatography (800 g of silica gel, 15% ethyl acetate in heptane) affording 38 g (94%) of the title compound 15 as a slightly yellow oil. ¹H NMR (CDCl₃): δ 9.56 (1H, d, J=2.0 Hz), 5.20 (1H, br s), 2.44-2.16 (1H, m), 2.03 (3H, s), 2.00-1.15 (12H, m), 1.11 (3H, d, J=7.0 Hz), 0.92 (3H, s).

### Acetic acid (3aR,4S,7aR)-1-E-ethylidene-7a-methyl-octahydroinden-4-yl ester (16)

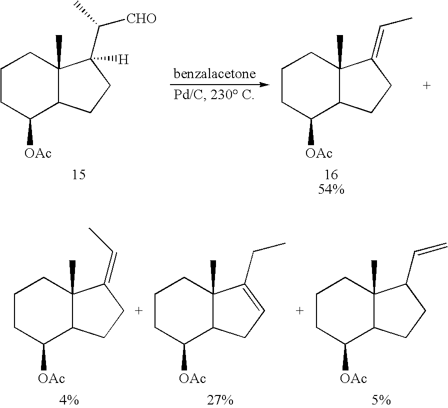

Benzalacetone was purified by bulb to bulb distillation (130° C., 10⁻² mbar) before use. To a solution of acetic acid (1R,3aR,4S,7aR)-7a-methyl-1-((S)-oxopropan-2-yl)-octahydro-1H-inden-4-yl ester 15 (38.3 g, 0.15 mol) in diethyl ether (240 mL) was added 10% palladium on charcoal (1.8 g). The suspension was stirred at room temperature for 45 min., filtered through a path of Celite and the filtrate was concentrated in vacuo. To the residue was added benzalacetone (28.3 g, 0.19 mol) and 10% palladium on charcoal (1.8 g). The suspension was degassed by evacuating the flask and refilling with nitrogen. Then the flask was partially immersed in a 230° C. oil bath for 40 min. After cooling at room temperature the suspension was diluted with ethyl acetate, filtered through a path of Celite and the filtrate was concentrated in vacuo. The residue was purified by column chromatography (1800 g of SiO₂, 5-10% ethyl acetate in heptane) affording 21.6 g (65%) of a mixture of Δ^{17E}, Δ^{17Z}, Δ¹⁶ and Δ²⁰ indene olefins, which are present in 51%, 4%, 25%, and 1%, respectively (GC analysis). The mixture of isomers was used in the next step without further purification.

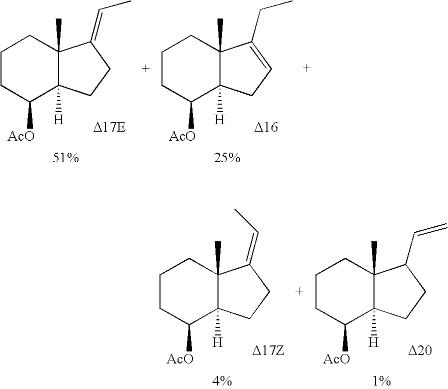

¹H NMR (CDCl₃, signals of the desired Δ^{17E} isomer): 5.21 (m, 1H), 4.98-5.07 (m, 1H), 2.15-2.35 (m, 2H), 2.05 (s, 3H), 1.53 (d, 3H, J=7 Hz), δ 0.96 (s, 3H).

In a different experiment the desired product was isolated from the mixture of olefins (Δ^{17E}:Δ^{17Z}:Δ⁶:Δ²⁰: =65:4:27:4) by silver nitrate impregnated silica gel medium pressure chromatography in a 55% yield (U.S. Pat. No. 5,939,408).

### (2R,3aR,4S,7aR)-1-E-ethylidene-2-hydroxy-7a-methyl-octahydroinden-4-yl ester (17a) and acetic acid (2S,3aR,4S,7aR)-1-E)-ethylidene-2-hydroxy-7a-methyl-octahydroinden-4-yl ester (17b)

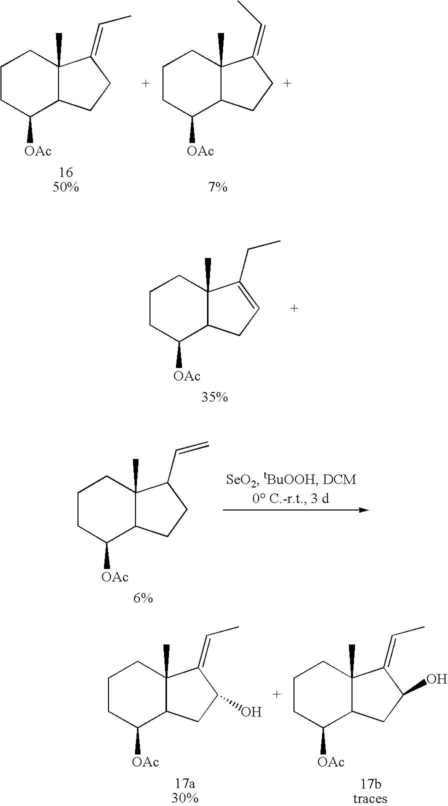

To a suspension of SeO₂ (1.4 g; 12.6 mmol) in dichloromethane (55 mL) was added t.-butylhydroperoxide (17 mL, 70 w/w-% solution in water, 124 mmol). The suspension was stirred at room temperature for 30 min, cooled at 0° C. and a solution of acetic acid (3aR,4S,7aS,E)-1-ethylidene-7a-methyl-octahydro-1H-inden-4-yl ester 16 (18.8 g, 84.5 mmol, as part of a mixture of Δ^{17E}, Δ^{17Z}, Δ¹⁶ and Δ²⁰ indene olefins; contains 51% of desired isomer 16) in dichloromethane (70 mL) was added dropwise. The reaction mixture was stirred at 0° C. for 1 h, at room temperature for 18 h and subsequently at 30° C. for 3 days. To the reaction mixture was added water (350 mL) and ethyl acetate (400 mL). The layers were separated and the aqueous layer was extracted with ethyl acetate (1×400 mL, 1×350 mL, 1×150 mL). Water (600 ml) was added to the combined organic fractions and the layers were mixed thoroughly for 60 min by magnetic stirring. The organic layer was separated, dried (Na₂SO₄) and concentrated in vacuo. The residue was purified by column chromatography (1 kg SiO₂; eluting with 4 L 20% AcOEt in heptane, 4 L 25% AcOEt in heptane, 4 L 33% AcOEt in heptane) affording: Fraction A (4.2 g, mixture containing ca. 75% of a ketone fragment); Fraction B (7.2 g of alcohol 16, purity ca. 90%). Fraction A was dissolved in methanol (100 mL) and cooled at 0° C. Sodium borohydride (1.1 g, 29 mmol) was added in portions. After stirring at 0° C. for 40 min., tlc showed complete conversion. The reaction mixture was quenched by addition of sat. aqueous NH₄Cl solution (30 mL) and was extracted with ethyl acetate (3×). The combined organic layers were washed with brine, dried (Na₂SO₄), filtered and the filtrate was concentrated in vacuo. The residue (4.5 g) was purified by column chromatography (SiO₂, ethyl acetate/heptane=1:3) to give: Fraction C (3.2 g, of alcohol 17b). Fraction B and C were combined affording 10.4 g of a mixture of alcohol 17a and 17b (93% yield based on the amount of 51% of 165 in the mixture of CD olefins) as a colorless oil.

Alcohol 17a: ¹H NMR (CDCl₃): δ 5.47 (qd, J=7.2, 1.2 Hz, 1H), 4.80 (br. s, 1H), 5.23 (m, 1H), 1.80-1.94 (m, 4H), 2.02 (s, 3H), 1.77 (dd, J=7.2, 1.2 Hz, 3H), 1.46-1.80 (m, 4H), 1.40-1.46 (m, 1H), 1.30 (m, 1H), 0.94 (s, 3H); GC-MS: m/e 223 (M−15), 178 (M−60), 163 (M−75); MS: m/e 223 (M−15), 178 (M−60), 163 (M−75).

Alcohol 17b: ¹H NMR (CDCl₃): δ 5.30 (qd, J=7.2, 1.2 Hz, 1H), 5.14 (m, 1H), 4.36 (m, 1H), 2.26 (m, 1H), 2.03 (s, 3H), 1.99 (ddd, J=11.0, 7.0, 3.7 Hz, 1H), 1.72 (dd, J=7.2, 1.2 Hz, 3H), 1.68-1.88 (m, 3H), 1.38-1.60 (m, 5H), 1.24 (s, 3H); GC-MS: m/e 223 (M−15), 178 (M−60), 163 (M−75); MS: m/e 223 (M−15), 178 (M−60), 163 (M−75).

### Acetic acid (3aR,4S,7aR)-7a-methyl-1-(1-(R)-methyl-3-oxo-propyl)-3a,4,5,6,7,7a-hexahydro-3H-inden-4-yl ester (18)

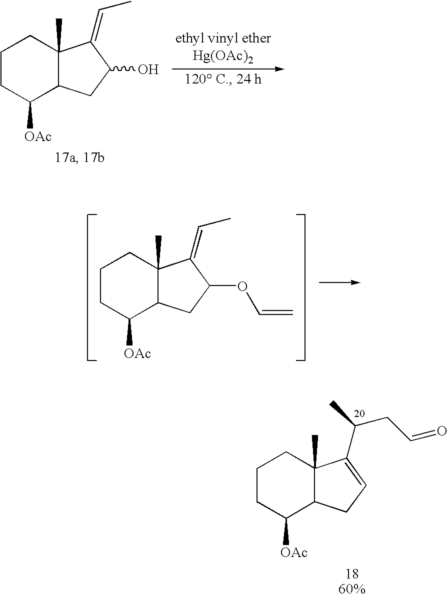

A mixture of acetic acid (2R,3aR,4S,7aR,Z)-1-ethylidene-2-hydroxy-7a-methyl-octahydro-1H-inden-4-yl ester 17a and acetic acid (2S,3aR,4S,7aS,Z)-1-ethylidene-2-hydroxy-7a-methyl-octahydro-1H-inden-4-yl ester 17b (12.5 g, 47 mmol) was dissolved in ethyl vinyl ether (150 mL). Hg(OAc)₂ (14.1 g, 44 mmol) was added and the suspension was poured into a pyrex pressure tube, flushed with N₂ and closed tightly. The mixture was stirred at 130° C. for 18 h, cooled at room temperature and concentrated in vacuo. The residue was purified by column chromatography (SiO₂, 7.5-30% ethyl acetate in heptane) to give: Fraction A (8.1 g (65%) of aldehyde 18); Fraction B (1.8 g, mixture containing ca 50% of aldehyde 18). Fraction B was purified by column chromatography (SiO₂, 7.5-30% ethyl acetate in heptane) to give: Fraction C (0.6 g of aldehyde 18). Fraction A and C were combined affording 8.7 g (70%) of 18 as a colorless oil. ¹H NMR (CDCl₃): δ 9.68 (s, 1H), 5.40 (m, 1H), 5.19 (m, 1H), 2.72 (m, 1H), 2.53 (ddd, J=16.2, 5.8, 1.8 Hz, 1H), 2.33 (ddd, J=16.2, 7.3, 2.6 Hz, 1H), 2.03 (s, 3H), 2.02-2.14 (m, 2H), 1.72-1.90 (m, 4H), 1.47-1.62 (m, 2H), 1.36 (M, 1H), 1.14 (d, J=7.1 Hz, 3H), 1.02 (s, 3H).

### 5(R)-((3aR,4S,7aR)-4-acetoxy-7a-methyl-3a,4,5,6,7,7a-hexahydro-3H-inden-1-yl)-hex-2-E-enoic acid ethyl ester (19)

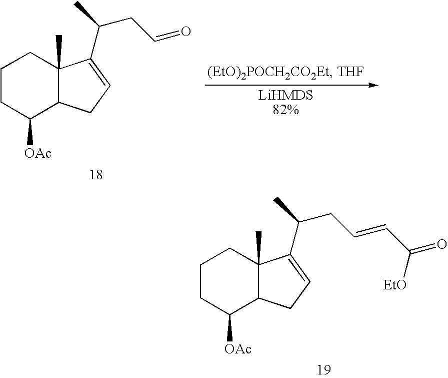

Acetic acid (3aR,4S,7aS)-7a-methyl-1-((S)-4-oxobutan-2-yl)-3a,4,5,6,7,7a-hexahydro-3H-inden-4-yl ester 18 (16.2 g; 61 mmol) and triethyl phosphonoacetate (36 ml; 183 mmol, 3 eq.) were dissolved under N₂ atmosphere in THF (200 mL, freshly distilled over Na/benzophenone). The mixture was cooled to −90° C. and a solution of LiHMDS in hexanes (122 mL, 1 M solution, 2 eq.) was added dropwise within 45 min. keeping the temperature below −90° C. After complete addition the reaction mixture was allowed to warm to −78° C. and stirring was continued at this temperature for 70 min. The reaction was quenched by dropwise addition of a mixture of water (64 ml) and sat. NH₄Cl solution (32 mL). To the reaction mixture was added tert-butyl methyl ether (400 ml) and water (400 mL), the organic layer was separated and concentrated in vacuo affording fraction A. The aqueous layer was extracted with tert-butyl methyl ether (1×400 ml, 1×200 ml). The organic layers were combined with fraction A, washed with water (2×200 ml), washed with brine (1×150 ml), dried (Na₂SO₄), filtered and the filtrate was concentrated in vacuo. The residue was purified by column chromatography (SiO₂, ethyl acetate/heptane=1:10) affording the title compound 19 (18 g, 88%) as a E/Z-mixture (E:Z=10:1). ¹H NMR (CDCl₃): δ 6.88 (dt, J=15.4, 7.3 Hz, 1H), 5.78 (dm, J=15.4 Hz, 1H), 5.37 (br. s, 1H), 5.20 (br. s, 1H), 4.17 (q, J=7.2 Hz, 2H), 2.03 (s, 3H), 2.22-2.39 (m, 2H), 1.96-2.17 (m, 3H), 1.72-1.90 (m, 4H), 1.46-1.62 (m, 2H), 1.36 (td, J=13.3, 4.0 Hz, 1H), 1.27 (t, J=7.1 Hz, 3H), 1.06 (d, J=7.2 Hz, 3H), 0.99 (s, 3H); MS: m/e 357 (M+23), 275 (M−59).

### (3aR,4S,7aR)-1-((S,E)-5-ethyl-5-hydroxy-1-methyl-hept-3-enyl)-7a-methyl-3a,4,5,6,7,7a-hexahydro-3H-inden-4-ol (20)

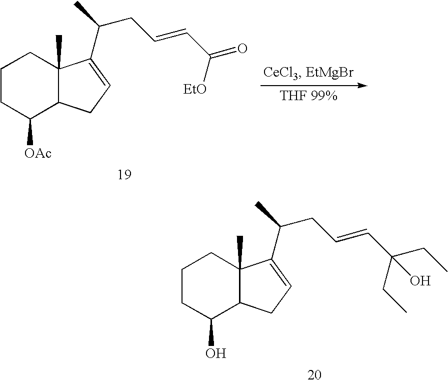

A 1 L round bottom flask was charged with cerium(III) chloride heptahydrate (234 g, 0.63 mol) and water (ca. 70 g) was removed in vacuo (10⁻² mbar) via bulb to bulb distillation by heating slowly at 70° C. (30 min), 95° C. (3 h), 120° C. (1 h) and 160° C. (3 h), respectively. After cooling overnight and under vacuo at room temperature the off-white cerium(III) chloride monohydrate (162 g) was transferred into a 3 L three-necked flask equipped with a magnetic stirring bar. The last equivalent of water was removed by stirring and heating in vacuo (10⁻² mbar) at 90° C. (1 h), 120° C. (1 h), 160° C. (1 h) and 210° C. (4 h), respectively. Condensate water on top of the flask was removed by heating with a hot gun. When no more formation of condensate was observed, removal of water was complete. The flask was cooled at room temperature and flushed with nitrogen. THF (1.3 L) was added and the mixture was stirred at room temperature for 18 h. The milky suspension was cooled at 0° C. and a solution of EtMgBr in THF (610 mL, 1 M solution) was added dropwise within 1 h. After stirring at 0° C. for 2 h a solution of (S,E)-5-((3aR,4S,7aS)-4-acetoxy-7a-methyl-3a,4,5,6,7,7a-hexahydro-3H-inden-1-yl)-hexenoic acid ethyl ester 19 (16.2 g, 48.4 mmol, contaminated with ca. 10% of the corresponding Z-isomer) in THF (75 mL) was added dropwise within 1 h. After stirring at 0° C. for 1 h tlc showed complete conversion and the reaction was quenched by slow addition of water (150 mL, exothermic reaction), upon which a sticky solid precipitated. The solution (Fraction A) was decanted and the residual solid was mixed thoroughly with water (1 L) to give an aqueous suspension (Fraction B). Fraction A and B were combined and extracted four times with a mixture of ethyl acetate (500 mL) and heptane (500 mL). The combined organic layers were washed with sat. NaHCO₃ solution (2×), brine (1×), dried (Na₂SO₄), filtered and the filtrate was concentrated in vacuo. The residue (17 g) was purified by column chromatography (1 kg SiO₂, 20% ethyl acetate in heptane) affording the title compound (13.4 g, 98%) as a slightly yellow oil. Purity according HPLC: 93.1% (λ=212 nm). The product was purified again by column chromatography (1 kg SiO₂, 20% ethyl acetate in heptane) to give: Fractions A 11.91 g, (86% yield) of 20 as a colorless oil; purity according HPLC: >96.5% (λ=212 nm); Fraction B 1.40 g, (10% yield) of 20 as a colorless oil; purity according HPLC: 86.9% (λ=212 nm); ¹H NMR (CDCl₃): δ 5.51 (ddd, J=15.4, 7.4, 6.5 Hz, 1H), 5.35 (dm, J=15.4 Hz, 1H), 5.33 (m, 1H), 4.17 (m, 1H), 2.12-2.32 (m, 3H), 1.67-2.02 (m, 6H), 1.23-1.60 (m, 9H), 1.05 (s, 3H), 1.04 (d, J=7.2 Hz, 3H), 0.84 (t, J=7.3 Hz, 6H); MS: in/e 329 (M+23), 289 (M−17), 271 (M−35).

### (3aR,4S,7aR)-1-((S,E)-5-ethyl-5-hydroxy-1-methyl-hept-3-enyl)-7a-methyl-3a,4,5,6,7,7a-hexahydro-3H-inden-4-one (5)

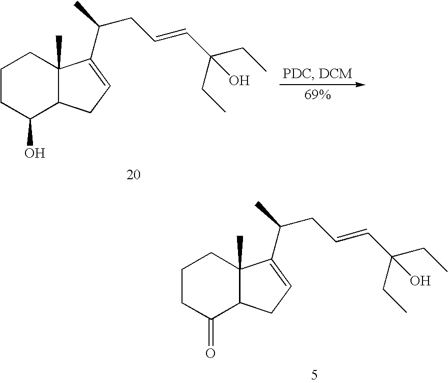

A solution of (3aR,4S,7aS)-1-((S,E)-6-ethyl-6-hydroxyoct-4-en-2-yl)-7a-methyl-3a,4,5,6,7,7a-hexahydro-3H-inden-4-ol 20 (4.70 g, 15.3 mmol, purity according HPLC: 96.5% (λ=212 nm) in dichloromethane (200 mL) was cooled in an ice-bath and treated portionwise with pyridinium dichromate (13.1 g, 34.9 mmol, 2.2 eq.). The reaction mixture was allowed to warm at room temperature overnight, filtered through a path of Celite and the filtercake was washed with dichloromethane. The combined filtrates were washed with a 2 M KHCO₃ solution, washed with brine, dried (Na₂SO₄) and concentrated in vacuo. the residue was purified by column chromatography (SiO₂, 25% ethyl acetate in heptane) affording the title compound 5 (4.0 g, 85%) as a colorless oil. ¹H NMR (CDCl₃): δ 5.54 (ddd, J=15.6, 7.1, 6.0 Hz, 1H), 5.38 (dm, J=15.6 Hz, 1H), 5.30 (m, 1H), 2.82 (dd, J=10.4, 6.0 Hz, 1H), 2.42 (ddt, J=15.4, 10.4, 1.6 Hz, 1H), 2.16-2.33 (m, 4H), 1.93-2.16 (m, 4H), 1.84-1.93 (m, 1H), 1.65 (td, J=12.1, 5.6 Hz, 1H), 1.52 (br. q, J=6.9 Hz, 4H), 1.34 (br. s, 1H), 1.05 (d, J=6.9 Hz, 3H), 0.85 (br. t, J=7.2 Hz, 6H), 0.82 (s, 3H).

### Example 4

### 1. Coupling and Synthesis of 1

### 1-(5-Ethyl-1-methyl-5-trimethylsilanyloxy-hept-3-enyl)-7a-methyl-3,3a,5,6,7,7a-hexahydro-4-inden-4-one (22)

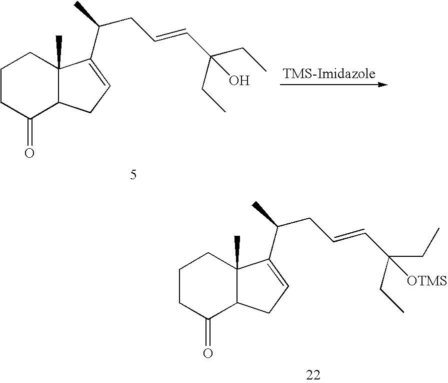

To a solution of compound 5 (320 mg, 1.05 mmol) in dichloromethane (20 mL) was added 1-(trimethylsilyl)imidazole (0.2 mL, 1.34 mmol). The reaction mixture was stirred at room temperature for 4 d. Reaction control (tlc) showed complete conversion. The mixture was concentrated in vacuo and the residue was purified by column chromatography (SiO₂, 10% ethyl acetate in heptane) affording compound 22 (377 mg, 95%) as a colorless oil.

### 1α-Fluoro-25-hydroxy-16-23E-diene-26,27-bishomo-20-epi-cholecalciferol (1)

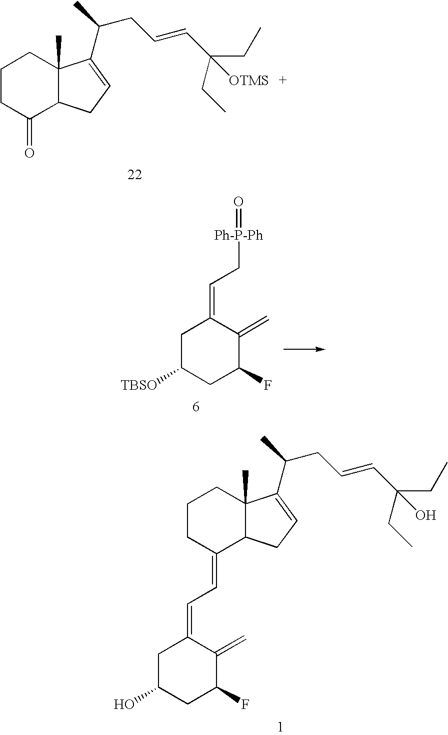

To a stirred solution of 240 mg (0.51 mmole) of 6 in 5 ml of anhydrous tetrahydrofuran at −78° C. was added 0.319 ml (0.51 mmole) of 1.6M n-butyllithium in hexane, dropwise under argon. After stirring for 5 min, to thus obtained red solution was added a solution of 103 mg (0.273 mmole) of 22 in 4 ml of anhydrous tetrahydrofuran, dropwise over a 10 min period. The reaction mixture was stirred at −78° C. for 2 hrs, then placed in freezer (−20° C.) for one hour, quenched by addition of 10 ml of a 1:1 mixture of 2N Rochelle salt and 2N potassium bicarbonate and warmed up to room temperature. After dilution with additional 25 ml of the same salts mixture, it was extracted with 3×90 ml of ethyl acetate. The combined organic layers were washed three times with water and brine, dried over sodium sulfate and evaporated to dryness. The residue was purified by FLASH chromatography on a 30 mm×7″ silica gel column with hexane-ethyl acetate (1:4), to give 145 mg of disilylated title compound. To a solution of 145 mg of disilyl intermediate in 3 ml anhydrous tetrahydrofuran was added 1.7 ml (1.7 mmole) of 1M tetrabutyl-ammonium fluoride in tetrahydrofuran under argon. The reaction mixture was stirred at room temperature for 18 hrs, and then quenched by addition of 10 ml water and stirring for 15 min. It was diluted with 20 ml of water and brine and extracted with 3×80 ml ethyl acetate. The organic layers were washed four times with water and brine, dried over sodium sulfate, and evaporated to dryness. The crude product was purified by FLASH chromatography on a 30 mm×5″ silica gel column with hexane-ethyl acetate (3:2), and by HPLC on a YMC 50 mm×50 cm silica gel column with hexane-ethyl acetate (1:1). It gave 90 mg (74%) of the title compound, crystallization from methyl acetate-hexane.

### 2. Larger Scale Coupling and Synthesis of 1

### 1-(5-Ethyl-1-methyl-5-trimethylsilanyloxy-hept-3-enyl)-7a-methyl-3,3a,5,6,7,7a-hexahydro-inden-4-one (22)

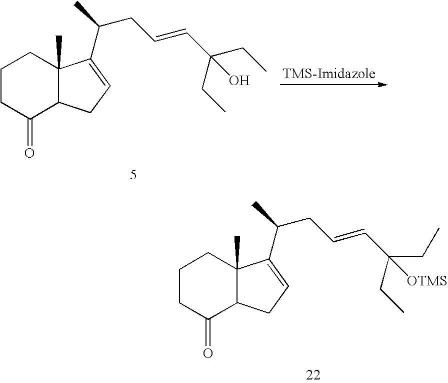

To a solution of (3aR,7aS)-1-((S,E)-6-ethyl-6-hydroxyoct-4-en-2-yl)-7a-methyl-3,3a,5,6,7,7a-hexahydro-3H-inden-4-one (5) (4.0 g, 13.1 mmol) in dichloromethane (200 mL) was added 1-(trimethylsilyl)imidazole (2.2 mL, 14.9 mmol). The reaction mixture was stirred at room temperature for 18 h. According tlc conversion was not complete and additional 1-(trimethylsilyl)imidazole (4.3 mL, 29.1 mmol) was added and stirring was continued for 5 h. The mixture was concentrated in vacuo at 30° C. and the residue was purified by column chromatography (200 g SiO₂, 10% ethyl acetate in heptane) affording the title compound 22 (4.6 g, 93%) as a colorless oil. Purity according HPLC: 100% (λ=265 nm); ¹H NMR (CDCl₃): δ 5.28-5.52 (m, 3H), 2.83 (dd, J=10.4, 6.1 Hz, 1H), 2.43 (ddm, J=15.4, 10.4 Hz, 1H), 2.18-2.32 (m, 4H), 1.94-2.18 (m, 4H), 1.85-1.93 (m, 1H), 1.76 (td, J=12.4, 5.6 Hz, 1H), 1.53 (br. q, J=7.3 Hz, 4H), 1.16 (d, J=6.9 Hz, 3H), 0.83 (s, 3H), 0.81 (br. t, J=7.1 Hz, 6H), 0.47 (s, 9H); MS: m/e 376 (M), 361 (M−15), 347 (M−29).

### 1α-Fluoro-25-hydroxy-16-23E-diene-26,27-bishomo-20-epi-cholecalciferol (1)

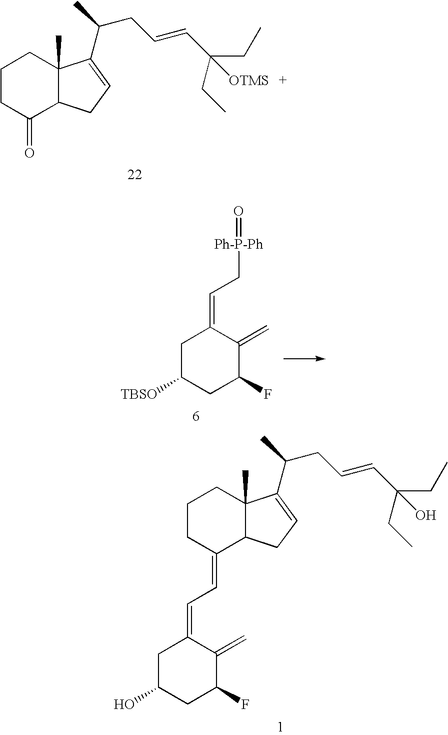

A 25 ml flask was charged with (1S,3Z,5R)-1-Fluoro-5-(tert-Butyldimethyl)silanyloxy)-2-methenyl-3-(diphenylphosphinoyl)ethylidene cyclohexane 6 (748 mg, 1.59 mmol, 1.2 eq) and (3aR,7aS)-1-((S,E)-6-ethyl-6-(trimethylsilyloxy)oct-4-en-2-yl)-7a-methyl-3,3a,5,6,7,7a-hexahydro-3H-inden-4-one 22 (499 mg, 1.32 mmol). The mixture was co-evaporated with toluene (3×5 mL), dissolved in THF (10 mL, freshly distilled over Na/benzophenone) and cooled to −55° C. LiHMDS (1.65 mL, 1 M solution in THF, 1.2 eq.) was added dropwise within 5 min. The deep red solution was allowed to warm to −25° C. within 1.5 h. TBAF (9 mL, 1 M solution in THF) was added (color turns to orange) and the mixture was allowed to warm to room temperature overnight. The reaction was quenched by pouring slowly into an ice-cold 1 M aqueous solution of KHCO₃. Thus formed mixture was extracted with ethyl acetate (3×25 mL). The combined organic layers were washed with water, brine (3×), dried (Na₂SO₄) and concentrated in vacuo at 30° C. The residue was purified by column chromatography (25% ethyl acetate in heptane), affording: Fraction A: 35 mg (7%) of epimerized CD-block epi-22. Fraction B: traces of Vitamin D-related byproducts. Fraction C: 27 mg (5%) of 1 as a white solid; purity according HPLC: 96.8% (λ=265 nm). Fraction D: 450 mg (75%) of 1 as a white solid; purity according HPLC: 93.7% (λ=265 nm). Fraction E: 30 mg (5%) of 1 as a white solid; purity according HPLC: 92.9% (λ=265 nm). Fraction D was dissolved in methyl formate (3-4 mL). Heptane (15 mL) was added and the flask was flushed with nitrogen gas until the solution became cloudy. The product started to crystallize and for complete crystallization the flask was stored at 4° C. for 1 h. The solvent was decanted and the remaining solid was washed with cold heptane (3×5 mL). After flushing with nitrogen gas the solid was dried in vacuo affording: Fraction F: 331 mg (56% yield) of 1 as a white solid; purity according HPLC: 100% (λ=265 nm); ¹H NMR (CD₃CN): δ 6.42 (br d, 1H), 6.10 (br d, 1H), 5.51 (ddd, 1H), 5.39 (br d, 1H), 5.36 (br s, 1H), 5.35 (br d, 1H), 5.13 (ddd, 1H), 5.07 (br s, 1H), 3.97-4.05 (m, 1H), 2.92 (d, 1H), 2.85 (dd, 1H), 2.57 (dd, 1H), 2.38 (dd, 1H), 2.14-2.29 (m, 5H), 1.96-2.04 (m, 2H), 1.84-1.89 (m, 1H), 1.73-1.82 (m, 3H), 1.64-1.72 (m, 1H), 1.53 (ddd, 1H), 1.45 (br. q, 4H), 1.04 (d, 3H), 0.81 (t, 6H), 0.69 (s, 3H); ¹³C NMR (CD₃CN): 160.12, 143.37 (d, J=17 Hz), 142.83, 137.33, 133.21 (d, J=2 Hz), 126.96, 124.84, 120.83, 117.33 (d, J=32 Hz), 115.40 (d, J=10 Hz), 93.74, 91.51, 74.83, 65.72 (d, J=5 Hz), 58.19, 50.31, 45.14, 40.94 (d, J=21 Hz), 39.78, 35.21, 33.34, 33.33, 32.46, 29.33, 28.63, 23.56, 20.33, 16.74, 1.41. ¹⁹F NMR (CD₃CN): δ −177.55; MS: m/e 482 (M+39), 465 (M+23), 425 (M−17). UV λmax: 244 nm (ε 13747), 270 nm (ε 13756) (CH₃OH). [α]^{D}₂₅ +101 (c 1.92, CH₃OH).

### Example 5

Alternate Coupling and Synthesis of 1

### 1α-Fluoro-25-hydroxy-16-23E-diene-26,27-bishomo-20-epi-cholecalciferol (1)

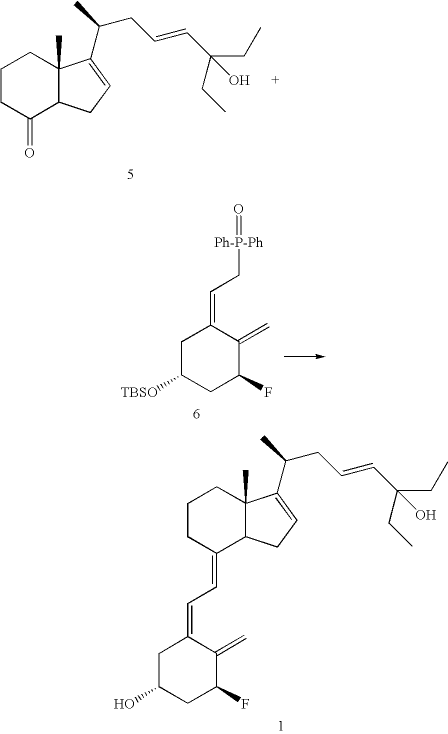

A solution of 6 (278 mg, 0.59 mmol, 3.6 eq.) in THF (10 mL, distilled over Na-benzophenone) was cooled at −75° C. and n-BuLi (0.23 mL, 2.5 M solution in hexanes, 0.57 mmol) was added dropwise. The red solution was stirred for 20 min. during which the temperature was allowed to rise to −50° C. A solution of 5 (50 mg, 0.164 mmol) in THF (2 mL, distilled over Na-benzophenone) was added dropwise at −50° C. within 5 min. Stirring was continued for 2 h during which the temperature was allowed to rise to −10° C. Tlc showed ca. 20% conversion. To the yellow solution was added dropwise TBAF (1.8 mL, 1 M solution in THF, containing ca. 5% water) upon which the solution turned red-brown. The reaction mixture was allowed to reach room temperature overnight. The reaction mixture was quenched by addition of an ice-cold aqueous 1 M KHCO₃ solution (3 g in 30 mL of water) and the mixture was extracted with ethyl acetate (2×40 mL). The combined organic layers were washed with water and brine, dried (Na₂SO₄), filtered and the filtrate was concentrated in vacuo at 30° C. The residue was purified by column chromatography (SiO₂, 25% ethyl acetate in heptane) affording 1 (13 mg, 18%) as a white foam.

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

1. A method of producing a vitamin D₃ compound of formula I 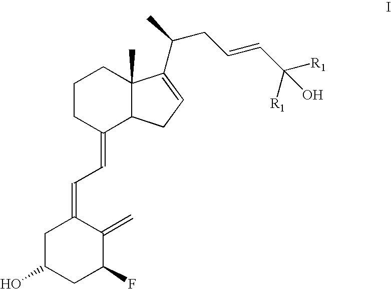
wherein:
each R₁ is independently alkyl;
and pharmaceutically acceptable esters, salts, and prodrugs thereof;
which comprises:
converting a compound of formula VI 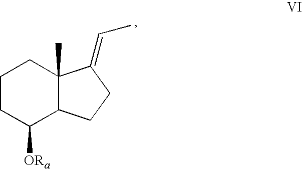
wherein Rₐ is a hydroxy protecting group, to a compound of formula X 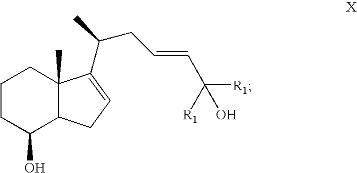
converting the compound of formula X to a compound of formula II 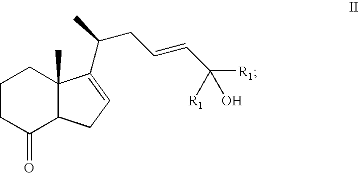
and
reacting the compound of formula II with a compound of formula III 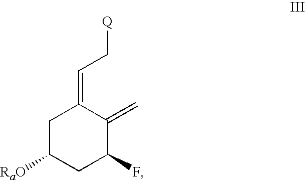
wherein Rₐ is defined as above and Q is a phosphorus-containing group;
to thereby produce a compound of formula I.

2. A method of producing a compound of formula X 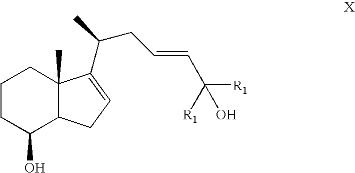
wherein:
each R₁ is independently alkyl;
which comprises:
converting a compound of formula VI 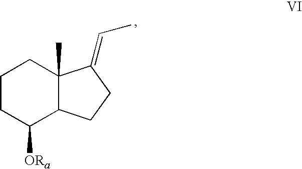
wherein Rₐ is a hydroxy protecting group, to a compound of formula VII 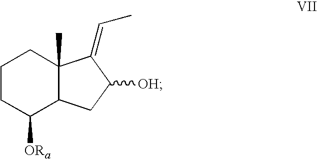
and
converting the compound of formula VII to a compound of formula X, to thereby produce a compound of formula X.

3. The method of claims 1 or 2, further comprising:
reacting the compound of formula VI 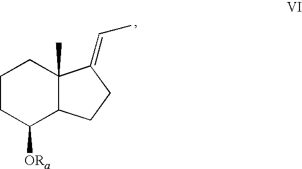
wherein Rₐ is a hydroxy protecting group, with an oxidation reagent to form a compound of formula VII

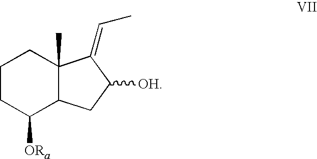

4. The method of claim 3, further comprising:
subjecting the compound of formula VII 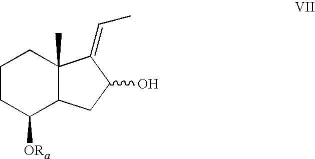
wherein Rₐ is a hydroxy protecting group;
to rearrangement conditions to form a compound of formula VIII

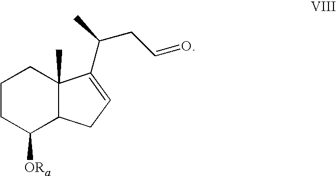

5. The method of claim 4, further comprising:
reacting the compound of formula VIII 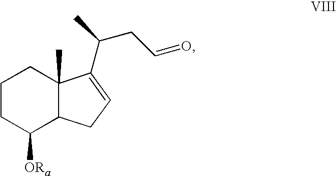
with a phosphorous-containing reagent of formula VIII-a 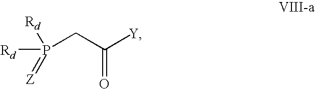
wherein Z is oxygen or absent; Y is OR_{b}, NR_{b}R_{b}, or S(O)ₙR_{b}; each R_{d} is independently alkyl, aryl, or alkoxy; each R_{b} is independently H, alkyl, or aryl; and n is 0-2; in the presence of a base to form a compound of formula IX 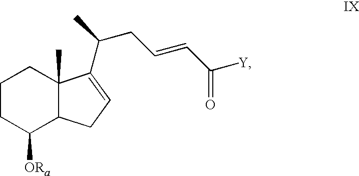
wherein: Rₐ and Y are as defined above.

6. The method of claim 5, further comprising:
reacting the compound of formula IX 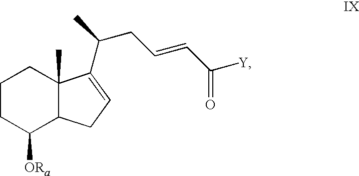
with an organometallic reagent to form a compound of formula X 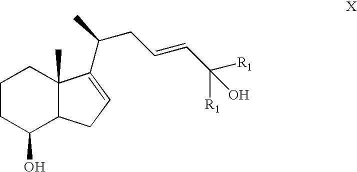
wherein each R₁ is independently alkyl.

7. The method of claim 3, wherein the oxidation reagent comprises selenium dioxide (SeO₂) and t-butylhydrogenperoxide.

8. The method of claim 4, wherein said rearrangement condition comprises Hg(OAc)₂.

9. The method of claim 5, wherein the phosphorus-containing compound of formula VIII-a is triethyl phosphonoacetate and the base is lithium hexamethyldisalazide (LiHMDS).

10. The method of claim 6, wherein the organometallic reagent is ethyl magnesium bromide (EtMgBr).

11. The method of claim 8, wherein the conversion takes place at a reaction temperature of about 120° C.

12. The method of claim 10, further comprising the addition of cerium trichloride (CeCl₃).

13. The method of claim 3, wherein the compound of formula VI is Acetic acid 1-ethylidene-7a-methyl-octahydro-inden-4-yl ester:

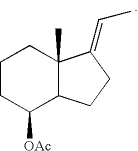

14. The method of claim 3, wherein the compound of formula VII is Acetic acid 1-ethylidene-2-hydroxy-7a-methyl-octahydro-inden-4-yl ester:

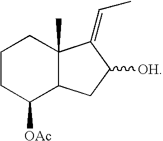

15. The method claim 4, wherein the compound of formula VIII is Acetic acid 7a-methyl-1-(1-methyl-3-oxo-propyl)-3a,4,5,6,7,7a-hexahydro-3H-inden-4-yl ester:

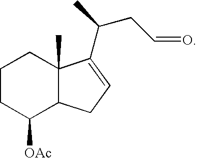

16. The method of claim 5, wherein the compound of formula IX is 5-(4-Acetoxy-7a-methyl-3a,4,5,6,7,7a-hexahydro-3H-inden-1-yl)-hex-2-enoic acid ethyl ester:

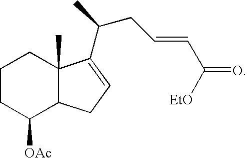

17. The method of claim 6, wherein the compound of formula X is 1-(5-Ethyl-5-hydroxy-1-methyl-hept-3-enyl)-7a-methyl-3a,4,5,6,7,7a-hexahydro-3H-inden-4-ol:

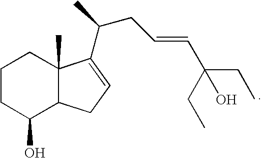

18. The method of claim 1, further comprising obtaining the compound of formula VI.

19. The method of claim 18, wherein the compound of formula VI is obtained by:
converting compound 3 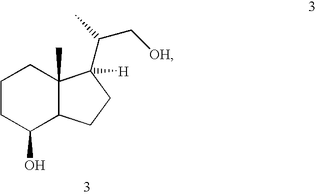
to compound 14 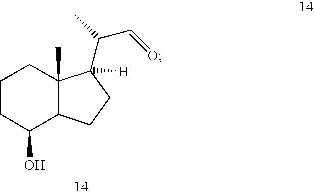
converting compound 14 to compound of formula XX 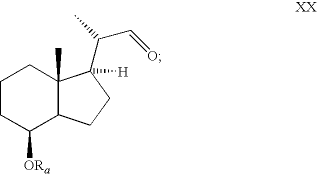
wherein Rₐ is a hydroxy protecting group;
and converting compound of formula XX to the compound of formula VI.

20. The method of claim 19, wherein the oxidation reagent for the conversion of 3 to 14 comprises TEMPO, tetrabutylammonium chloride hydrate and N-chlorosuccinimide.

21. The method of claim 19, wherein the compound of formula XX is Acetic acid 7a-methyl-1-(1-methyl-2-oxo-ethyl)-octahydro-inden-4-yl ester:

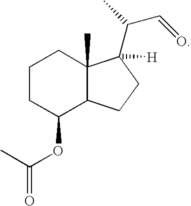

22. The method of claim 18, wherein the compound of formula VI is obtained by:
converting compound 3 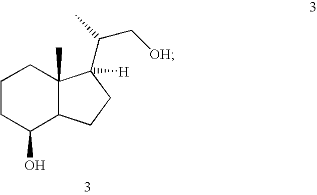
to a compound of formula XXI 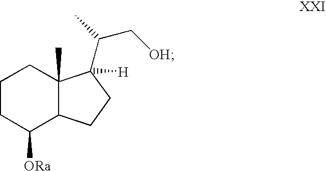
wherein Rₐ is a hydroxy protecting group;
converting a compound of formula XXI to a compound of formula XX 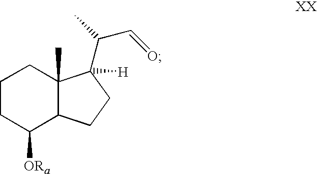
wherein Rₐ is a hydroxy protecting group;
and converting the compound of formula XX to the compound of formula VI.

23. The method of claim 22, wherein the oxidation reagent for the conversion of XXI to XX comprises oxalyl chloride.

24. The method of claim 22, wherein the compound of formula XXI is Acetic acid 1-(2-hydroxy-1-methyl-ethyl)-7a-methyl-octahydro-inden-4-yl ester:

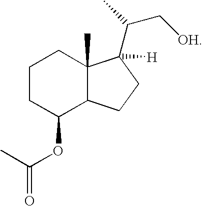

25. A method of producing a vitamin D₃ compound of formula I: 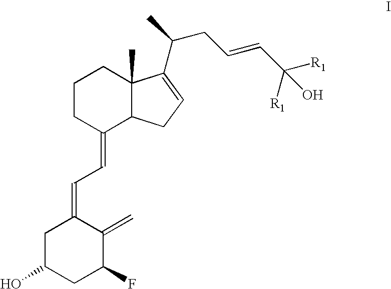
wherein:
each R₁ is independently alkyl;
and pharmaceutically acceptable esters, salts, and prodrugs thereof;
which comprises:
converting a compound of formula XII 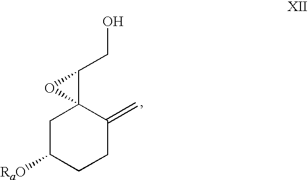
wherein Rₐ is a hydroxy protecting group, to a compound of formula XII-a 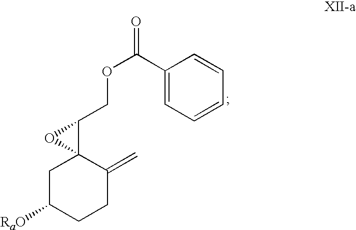
converting the compound of formula XII-a to a compound of formula XV 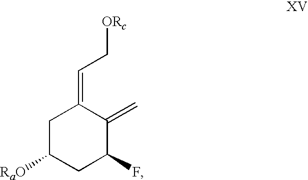
wherein R_{c} is H or benzoyl;
converting the compound of formula XV to a compound of formula III 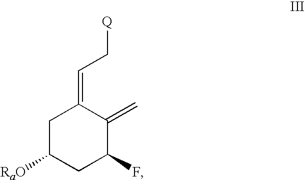
wherein,
Q is a phosphorus-containing group; and
reacting the compound of formula III with a compound of formula II 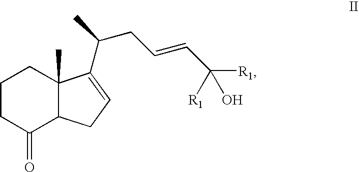
to thereby produce a compound of formula I.

26. A method of producing a compound of formula XV 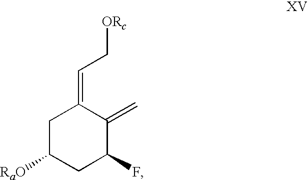
wherein R_{c} is H or benzoyl;
which comprises:
converting a compound of formula XII 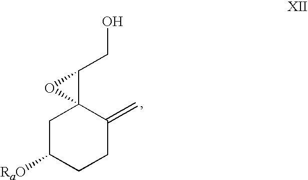
to a compound of formula XII-a 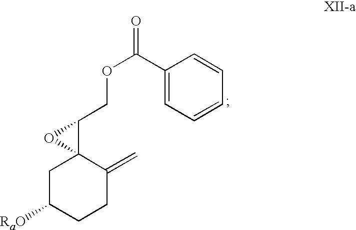
and
converting the compound of formula XII-a to a compound of formula XV, to thereby produce a compound of formula XV.

27. The method of claims 25 or 26, wherein the conversion of the compound of formula XII to the compound of formula XII-a is carried out in the presence of benzoyl chloride and base.

28. The method of claim 27, further comprising:
reacting the compound of formula XII-a 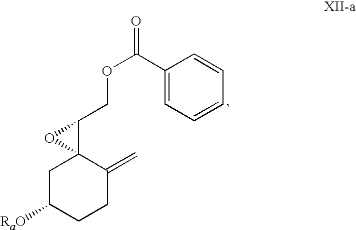
with an oxidizing agent, to provide a compound of formula XIII

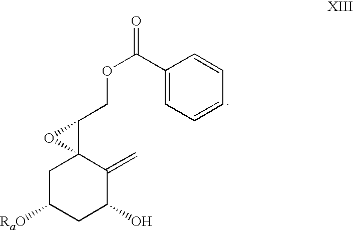

29. The method of claim 28, further comprising:
reacting the compound of formula XIII 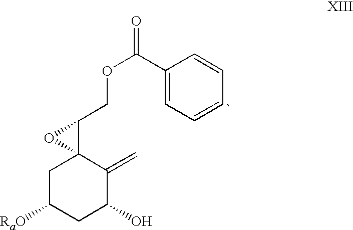
with a fluorinating agent, to provide a compound of formula XIV

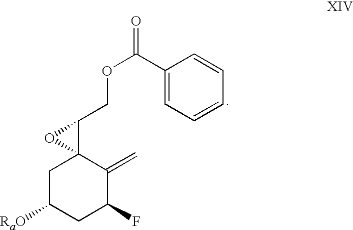

30. The method of claim 29, further comprising:
reacting the compound of formula XIV 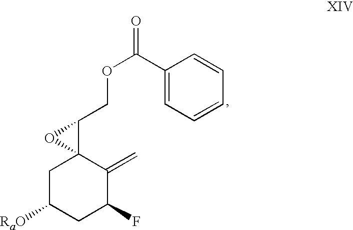
with a deoxygenation agent, to provide a compound of formula XV

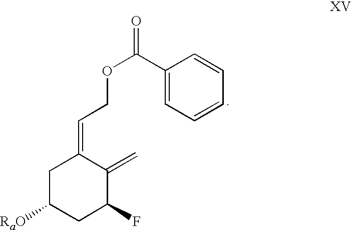

31. The method of claim 30, further comprising:
reacting the compound of formula XV 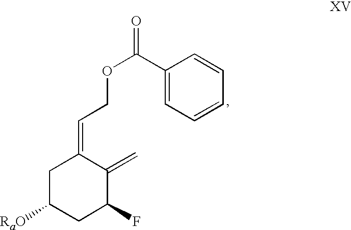
with a deprotection agent, to provide a compound of formula XV

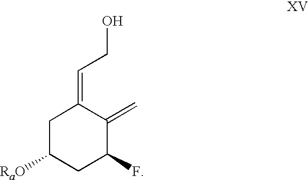

32. The method of claim 29, further comprising:
reacting the compound of formula XIV 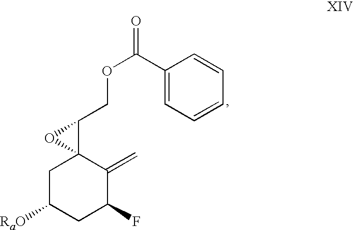
with a deoxygenation agent, to provide a compound of formula XVa

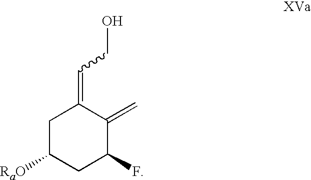

33. The method of claim 32, further comprising:
reacting the compound of formula XVa 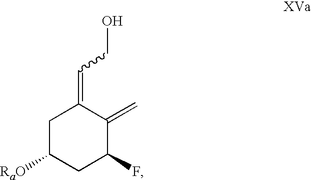
with an epimerizing agent, to provide a compound of formula XV

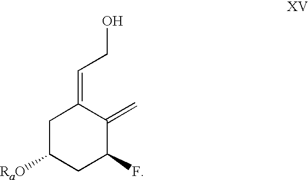

34. The method of claim 25, further comprising:
reacting the compound of formula XV 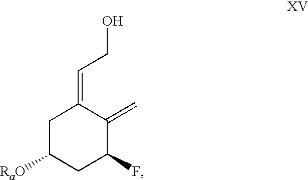
with a chlorinating agent, to provide a compound of formula XVI

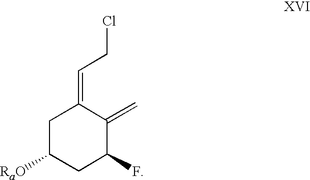

35. The method of claim 34, further comprising:
reacting the compound of formula XVI 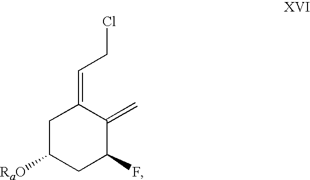
with a phosphorous containing agent in the presence of a base, to provide a compound of formula III

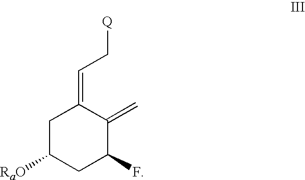

36. The method of claim 27, wherein the base is pyridine.

37. The method of claim 28, wherein the oxidizing reagent comprises selenium dioxide and t-butyl hydrogen peroxide.

38. The method of claim 29, wherein the fluorinating agent is diethylaminosulfur trifluoride (DAST).

39. The method of claim 30 or 31, wherein the deoxygenation reagent is tris(3,5-dimethylpyrazoyl)hydridoborate rhenium trioxide or tungsten hexachloride/nBuLi.

40. The method of claim 31, wherein the deprotection agent is sodium methoxide.

41. The method of claim 33, wherein the epimerization agent comprises hv and 9-fluorenone.

42. The method of claim 34, wherein the chlorinating agent comprises triphosgene and pyridine.

43. The method of claim 35, wherein the phosphorous containing agent is diphenyl phosphine oxide.

44. The method of claim 36, wherein the base is sodium hydride.

45. The method of claim 27, wherein the compound of formula XII-a is Benzoic acid 7-(tert-butyl-dimethyl-silanyloxy)-4-methylene-1-oxa-spiro[2.5]oct-2-ylmethyl ester:

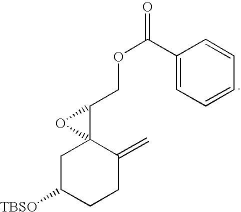

46. The method of claim 28, wherein the compound of formula XIII is Benzoic acid 7-(tert-butyl-dimethyl-silanyloxy)-5-hydroxy-4-methylene-1-oxa-spiro[2.5]oct-2-ylmethyl ester:

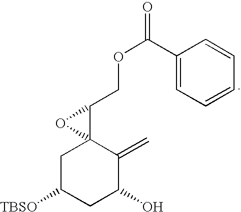

47. The method of claim 29, wherein the compound of formula XIV is Benzoic acid 7-(tert-butyl-dimethyl-silanyloxy)-5-fluoro-4-methylene-1-oxa-spiro[2.5]oct-2-ylmethyl ester:

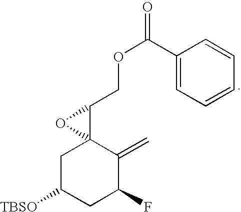

48. The method of claim 30, wherein the compound of formula XV is Benzoic acid 2-[5-(tert-butyl-dimethyl-silanyloxy)-3-fluoro-2-methylene-cyclohexylidene]-ethyl ester:

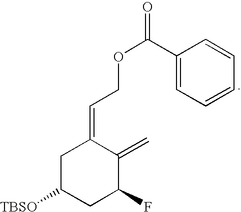

49. The method of claim 32, wherein the compound of formula XVa is 2-[5-(tert-Butyl-dimethyl-silanyloxy)-3-fluoro-2-methylene-cyclohexylidene]-ethanol:

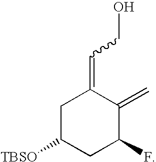

50. The method of claim 31 or 33, wherein the compound of formula XV is 2-[5-(tert-Butyl-dimethyl-silanyloxy)-3-fluoro-2-methylene-cyclohexylidene]-ethanol:

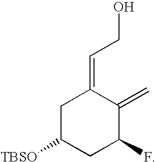

51. The method of claim 34, wherein the compound of formula XVI is tert-Butyl-[3-(2-chloro-ethylidene)-5-fluoro-4-methylene-cyclohexyloxy]-dimethyl-silane:

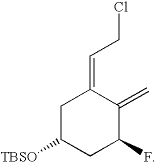

52. The method of claim 35, wherein the compound of formula III is tert-Butyl-[3-[2-(diphenyl-phosphinoyl)-ethylidene]-5-fluoro-4-methylene-cyclohexyloxy]-dimethyl-silane:

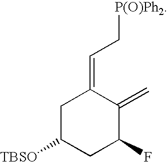

53. The method of claims 1 or 25, wherein the coupling reaction of the compound of formula II and the compound of formula III to form the compound of formula I comprises:
converting the compound of formula II 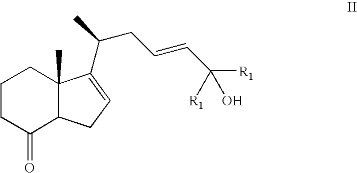
to a compound of formula XVII 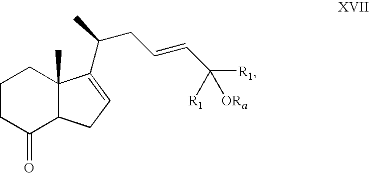
wherein Rₐ is hydroxy protecting group;
reacting the compound of formula XVII with a compound of formula III in the presence of base 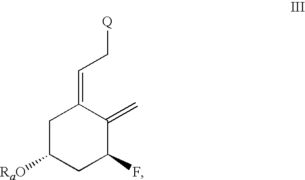
wherein Q is a phosphorus-containing group, to form a compound of formula XVIII 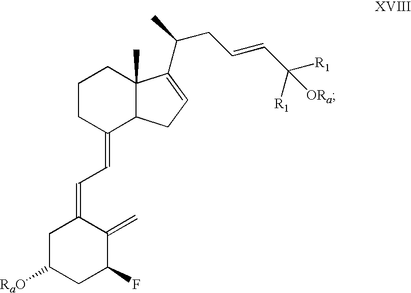
and converting the compound of formula XVIII to the compound of formula I.

54. The method of claims 1 or 25, wherein the reaction of the compound of formula II and the compound of formula III to produce the compound of formula I is carried out in a single process step.

55. The method of claim 53, wherein the compound of formula I is produced in 21 process steps.

56. The method of claim 54, wherein the compound of formula I is produced in 19 process steps.

57. The method of claims 1 or 25, wherein each R₁ is ethyl.

58. The method of claims 1 or 25, wherein the compound of formula I is

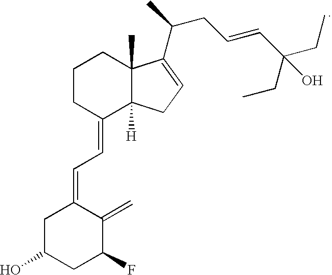

59. A method of producing a vitamin D₃ compound of formula I 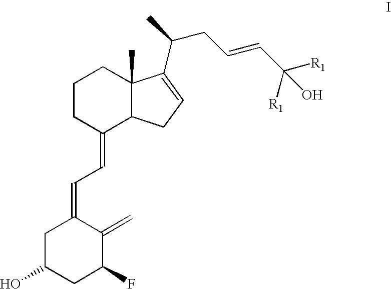
wherein:
each R₁ is independently alkyl;
and pharmaceutically acceptable esters, salts, and prodrugs thereof;
which comprises:
reacting a compound of formula II 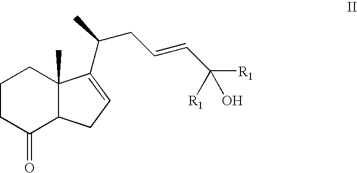
with a compound of formula III 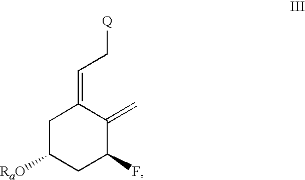
wherein Rₐ is defined as above and Q is a phosphorus-containing group in the presence of a strong base; to thereby produce a compound of formula I.

60. The method of claim 59, wherein the strong base is n-butyl lithium.

61. The method of claim 19, further comprising obtaining compound 3.

62. The method of claim 61, wherein compound 3 is obtained by:
converting compound 2 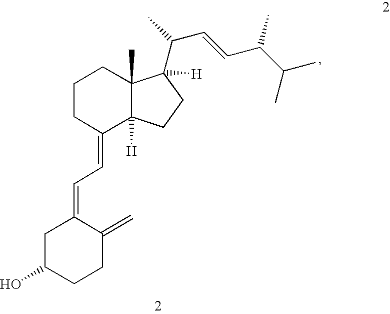
to compound 7 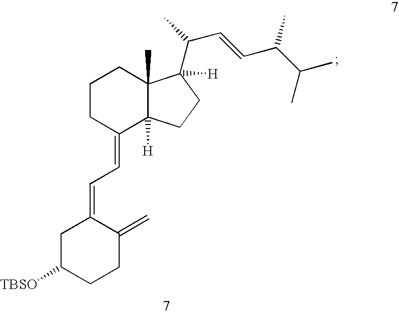
and converting compound 7 to compound 3.

63. The method of claim 25, further comprising obtaining the compound of formula XII.

64. The method of claim 63, wherein the compound of formula XII is obtained by:
converting compound 2 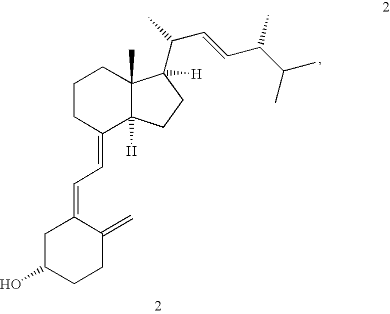
to compound 4a 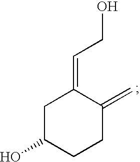
converting compound 4a to compound 4 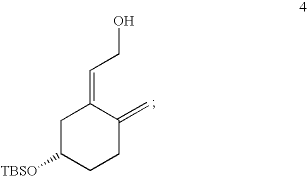
and converting compound 4 to the compound of formula XII.

65. The method of claim 64, wherein the epoxidation reagent comprises m-chloroperoxybenzoic acid (M-CPBA).

66. The compound Acetic acid 1-ethylidene-2-hydroxy-7a-methyl-octahydro-inden-4-yl ester:

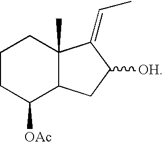

67. The compound Acetic acid 7a-methyl-1-(1-methyl-3-oxo-propyl)-3a,4,5,6,7,7a-hexahydro-3H-inden-4-yl ester:

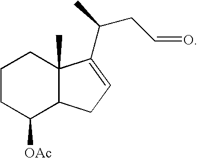

68. The compound 5-(4-Acetoxy-7a-methyl-3a,4,5,6,7,7a-hexahydro-3H-inden-1-yl)-hex-2-enoic acid ethyl ester:

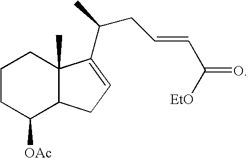

69. The compound Benzoic acid 7-(tert-butyl-dimethyl-silanyloxy)-5-fluoro-4-methylene-1-oxa-spiro[2.5]oct-2-ylmethyl ester:

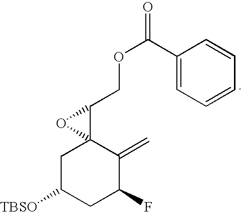

70. The compound Benzoic acid 2-[5-(tert-butyl-dimethyl-silanyloxy)-3-fluoro-2-methylene-cyclohexylidene]-ethyl ester:

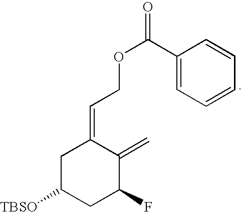

71. The compound Acetic acid 1-(2-hydroxy-1-methyl-ethyl)-7a-methyl-octahydro-inden-4-yl ester:

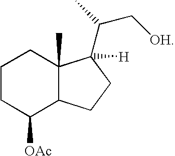

72. The compound 4-(tert-Butyl-dimethyl-silanyloxy)-2-[2-(tert-butyl-dimethyl-silanyloxy)-ethylidene]-1-methylene-cyclohexane:

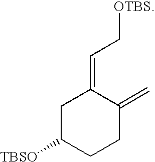

73. The method of claims 1 or 25, wherein the total synthesis of 1 is carried out in 19 steps.

74. The method of claim 1, further comprising obtaining any one of compounds II, III, VI or X.
